(19) United States
(12) Patent Application Publication
(10) Pub. No.: US 2023/0400474 A1
(43) Pub. Date: Dec. 14, 2023

(54) PROGRAMMABLE NANO-REACTORS FOR STOCHASTIC SENSING (PNRSS)

(71) Applicant: NANJING UNIVERSITY, Nanjing, Jiangsu (CN)

(72) Inventors: Shuo HUANG, Nanjing (CN); Wendong JIA, Nanjing (CN)

(21) Appl. No.: 18/252,672

(22) PCT Filed: Jul. 22, 2021
(86) PCT No.: PCT/CN2021/107874

§ 371 Date: May 11, 2023

Related U.S. Application Data
(63) Continuation-in-part of application No. PCT/CN2020/128706, filed on Nov. 13, 2020.

(51) Int. Cl. G01N 33/94; G01N 33/543; G01N 33/53

(52) U.S. Cl. CPC G01N 33/9406; G01N 33/54373; G01N 33/5308

(57) ABSTRACT

A system and a method for characterizing a target analyte are provided. The system comprises a nanopore and a polymer strand comprising a tether site and a reaction section, wherein the polymer strand is tethered via the tether site so that the polymer strand cannot pass through the nanopore, and wherein the reaction section comprises at least one sensing module which can interact with single molecule of the target analyte.

### FIELD OF THE INVENTION

The present invention relates to a system and a method for identifying an analyte using nanopore.

### BACKGROUND OF THE INVENTION

Though described and formulated in terms of single molecules, chemical reactions are rarely monitored or characterized at the single molecule level but in ensembles. Few state of the art instruments, including scanning probe microscopy¹, tip/surface enhanced Raman spectroscopy², molecular junctions³, single molecule force spectroscopy⁴ or biological nanopores 5 are capable of resolving discrete steps of the chemical reactions of a single molecule. These state-of-the-art techniques however report different aspects of single molecule properties. Scanning tunnelling microscopy and molecular junctions focus on the electronic state of the target molecule. Raman spectroscopy focuses on investigation of the vibration of chemical bonds. Whereas, force spectroscopy measures the elastic properties molecules.

Biological nanopores, including alpha-haemolysin (α-HL)⁶, Mycobacterium smegmatis porin A (MspA)⁷, aerolysine⁸, CsgG⁹, cytolysin A (ClyA)¹⁰, fragaceatoxin C (FraC)¹¹, pleurotolysin (PlyA/B)¹², outer membrane protein G (OmpG)¹³, phi29 connector¹⁴ and a few others, are a category of large channel proteins developed for single molecule sensing. Conformational properties of nucleic acids¹⁵, peptides¹⁶, proteins¹⁷ and small molecules¹⁸ can be probed during translocation of the analyte through the pore constriction. The nanopore sequencer MinION™, which applies a similar measurement scheme, is able to directly report nucleic acid sequences¹⁹. Pioneered by Bayley et al 5, an engineered α-HL nanopore which, with a sole, fixed reactive site within its lumen is capable of reacting with discrete freely translocating reactants, forming a single molecule reactor, capable to resolve binding of a single metal ion²⁰⁻²². However, nanopore single molecule chemistry measurements performed by α-HL generally report a weak event amplitude, measuring 1-5 pA²³, which prohibit it from gaining a further refined resolution. Undesired reactive sites evenly distributed within the cylindrical lumen of α-HL may also interfere with the measurement, requiring excessive pore engineering efforts²². Most biological nanopores show an oligomeric symmetry⁶ and introduction of a sole reactive site therefore requires a significant effort to produce a hetero-oligomeric assembly⁵, a niche technique mastered by only few in the field. Nanopore single molecule chemistry measurements performed by engineered homo-oligomeric porins will inevitably report simultaneous binding from multiple reactants, not suitable for event recognition and quantification²⁴⁻²⁷. Modifications in the pore lumen may interfere unpredictably with the assembly or the stability of the pore and consequently, a considerable effort of protein screening cannot therefore be avoided. To introduce unnatural reactive sites into the pore lumen, a semi-synthetic α-HL were prepared by native chemical ligation (NCL)^{28, 29}. Nanopore preparation by NCL however requires extremely complicated purification procedures and is not always guaranteed to work when engineering to a different site or pore type is to be performed. Some other state-of-the-art techniques apply internal adaptors such as cyclodextrins^{30,31} or proteins^{18,32} inside the pore lumen to acquire new sensing abilities. These approaches however require existence of suitable adaptors. Accommodation of large enzymatic adaptors may also report an undesired resolution to discriminate molecular analytes with extremely minor structural differences. A technical breakthrough to achieve a complete freedom in the placement of any type, number or spatial combination of reactive sites to any spot of the pore lumen is required to resolve these problems.

### SUMMARY OF THE INVENTION

The first aspect of the present invention provides a system for characterizing a target analyte, comprising:
a nanopore; and
a polymer strand comprising a tether site and a reaction section,
wherein the polymer strand is tethered via the tether site so that the polymer strand cannot pass through the nanopore, and wherein the reaction section comprises at least one sensing module which can interact with single molecule of the target analyte.

In some embodiments, the reaction section comprises two or more sensing modules which can interact with two or more different target analytes.

In some embodiments, each sensing module consists of one, two or more sensing moieties and each sensing moiety can interact with one or two or more binding sites of single molecule of the target analyte.

In some embodiments, the sensing moiety is selected from the group consisting of base of any nucleotide, any amino acid, 1,2,3-trizole, phenylboronic acid (PBA) or any combination thereof.

In some embodiments, at least one of the sensing modules consists of two neighbouring purines selected from the group consisting of guanine and adenine.

In some embodiments, the reaction section is prepared by any one of the following ways or any combination thereof:
a. incorporating one or more monomers comprising a sensing moiety to the reaction section;
b. incorporating one or more monomers comprising a functional group to the reaction section and chemically modifying the functional group to a sensing moiety; or
c. incorporating one or more monomers comprising a first reactive handle to the reaction section and reacting the first reactive handle to a second reaction handle which is linked to a sensing moiety.

In some embodiments, the first reactive handle and the second reaction handle are click reaction handles.

In some embodiments, the first reactive handle and the second reaction handle are selected from the group consisting of azide and alkyne.

In some embodiments, the polymer strand is tethered to a stopper molecule or the nanopore protein.

In some embodiments, the stopper molecule is a protein which can specifically bind a small molecule compound, the tether site comprises the small molecule compound, and the polymer strand is tethered to the stopper molecule through the specific binding of the small molecule compound to the protein.

In some embodiments, the stopper molecule is streptavidin or an antibody of a hapten and the small molecule compound is biotin or the hapten.

In some embodiments, the tether site comprises a small molecule that can react with a natural amino acid on the surface of the stopper molecule or the nanopore protein, and the polymer strand is tethered to the stopper molecule through the reaction between the small molecule compound and the natural amino acid.

In some embodiments, a first reactive handle is introduced to the surface of the stopper molecule or the nanopore protein, the tether site comprises a second reactive handle, and the polymer strand is tethered to the stopper molecule through the reaction between the first reactive handle and the second reactive handle.

In some embodiments, the polymer strand further comprises an extension section and the extension section is configured to enable the reaction section to be located in a region suitable for measurement of a blockage.

In some embodiments, the polymer strand further comprises a traction section and the traction section is configured to hold the reaction section in a region suitable for measurement of a blockage.

In some embodiments, the traction section comprises any one of the following:
a. a polymer chain which tend to pass through the nanopore channel in the electric field applied to the nanopore;
b. a coupling site which can react with a natural amino acid on the surface of the channel of the nanopore;
c. a second reactive handle which can react with a first reactive introduced to the surface of the channel of the nanopore; or
d. a polymer chain that can pass through the channel of the nanopore and form a three-dimension structure outside the nanopore which has a size larger than the exit opening of the nanopore.

In some embodiments, the traction section is a nucleic acid with a length of 10 nt or more.

In some embodiments, the polymer strand is based on nucleic acid, nucleic acid analog, polypeptide, polysaccharide, a homopolymer, a copolymer, or any combination thereof.

In some embodiments, the target analyte is selected from the group consisting of:
ion comprising metal element; preferably ion comprising alkaline-earth metal or transition metal; more preferably, AuCl₄⁻, Mg²⁺, Ca²⁺, Ba²⁺, Ni²⁺, Cu²⁺, Co²⁺, Zn²⁺, Cd²⁺, Ag²⁺ or Pb²⁺;
monosaccharide; preferably ribose, fructose or mannose; more preferably, D-(−)-ribose, D-fructose or D-(+)-mannose;
oligosaccharide; preferably disaccharide or trisaccharide; more preferably, 4-O-β-d-galactopyranosyl-d-fructofuranose (lactulose), 6-O-α-D-glucopyranosyl-D-fructofuranose (isomaltulose) or 4-O-b-D-galactosylsucrose (galactosylsucrose);
polysaccharide;
glucoside;
polyphenol, such as anthocyanin or proanthocyanidin;
catecholamine;
catecholamine derivative; preferably, epinephrine, norepinephrine or isoprenaline;
polyol; preferably compound containing two vicinal hydroxyl groups, a 1,2-cis-diol or a 1,3-cis-diol moiety; more preferably, 3,4-Dihydroxymandelic acid, 4-Hydroxy-3-methoxymandelic acid (VMA), 3,4-Dihydroxyphenylacetic acid, catechol, ethylene glycol, glycerol, L-lactic acid, or vitamin (such as vitamin C or vitamin B6);
protonated or deprotonated forms of a compound; preferably, protonated or deprotonated forms of tris;
a compound containing a ribose moiety; preferably, nucleotide, nucleoside, analog thereof or monophosphate derivative thereof or polyphosphate derivative thereof; more preferably, ribonucleotide, deoxyribonucleotide, galidesvir, ribavirin, favipiravir-RTP, remdesivir or its triphosphate metabolite, cytidine 5′-monophosphate (5′-CMP);
hydrogen peroxide;
oligopeptide or cyclopeptide;
buffer reagent; preferably, tris;
smaller molecular drug; preferably, nucleoside analogue medicines; more preferably, galidesvir, ribavirin, favipiravir-RTP, remdesivir or its triphosphate metabolite;
neurotransmitter; preferably, catecholamine or derivative thereof;
compound with a specific chirality; preferably, L-Norepinephrine or D-Norepinephrine;
analyte containing an isotope; preferably, catechol-D6 (deuterium replaces all the hydrogen atoms on catechol);
chemical intermediate;
or any combination thereof.

In some embodiments, the nanopore is a biological nanopore, a solid nanopore or a DNA nanopore.

In some embodiments, the protein nanopore is MspA, α-HL, Aerolysin, ClyA, FhuA, FraC, PlyA/B, CsgG Phi 29 connector or a homolog or variant thereof.

In some embodiments, the system comprises two or more nanopores.

Another aspect of the present invention provides a method for characterizing a target analyte, the method comprising:
(i) providing the system according to any one of claims 1-22;
(ii) applying a voltage between the two sides of the nanopore and allowing one polymer strand to enter the nanopore;
(iii) allowing a target analyte to pass through the nanopore; and
(iv) measuring an ionic current through the nanopore to provide a current pattern, and characterizing the target analyte based on the current pattern.

In some embodiments, the polymer strand of the system comprises two or more sensing modules which can interact with two or more different target analytes, and wherein the method is for characterizing two or more target analytes.

In some embodiments, the method comprises:
(i) providing the system according to any one of claims 1-22;
(ii) applying a first voltage between the two sides of the nanopore and allowing one polymer strand to enter the nanopore;
(iii) allowing a first target analyte to pass through the nanopore; and
(iv) measuring an ionic current through the nanopore to provide a current pattern, and characterizing the first target analyte based on the current pattern.
(v) switching the voltage between the two compartments to a second voltage which is in the opposite direction to the first voltage, thereby causing the polymer strand in the nanopore to exit from the nanopore;
(vi) switching the voltage between the two compartments to the first voltage and allowing another polymer strand to enter the nanopore; and
(vi) applying steps (iii)-(iv) to a second target analyte which is different from the first target analyte.

In some embodiments, the sensing module is capable of irreversibly interacting with the first target analyte and/or the second target analyte.

In some embodiments, the target analyte is selected from the group consisting of:

ion comprising metal element; preferably ion comprising alkaline-earth metal or transition metal; more preferably, AuCl₄⁻, Mg²⁺, Ca²⁺, Ba²⁺, Ni²⁺, Cu²⁺, Co²⁺, Zn²⁺, Cd²⁺, Ag²⁺ or Pb²⁺;
monosaccharide; preferably ribose, fructose or mannose; more preferably, D-(−)-ribose, D-fructose or D-(+)-mannose;
oligosaccharide; preferably disaccharide or trisaccharide; more preferably, 4-O-β-d-galactopyranosyl-d-fructofuranose (lactulose), 6-O-α-D-glucopyranosyl-D-fructofuranose (isomaltulose) or 4-O-b-D-galactosylsucrose (galactosylsucrose);
polysaccharide;
glucoside;
polyphenol, such as anthocyanin or proanthocyanidin;
catecholamine;
catecholamine derivative; preferably, epinephrine, norepinephrine or isoprenaline;
polyol; preferably compound containing two vicinal hydroxyl groups, a 1,2-cis-diol or a 1,3-cis-diol moiety; more preferably, 3,4-Dihydroxymandelic acid, 4-Hydroxy-3-methoxymandelic acid (VMA), 3,4-Dihydroxyphenylacetic acid, catechol, ethylene glycol, glycerol, L-lactic acid, or vitamin (such as vitamin C or vitamin B6);
protonated or deprotonated forms of a compound; preferably, protonated or deprotonated forms of tris;
a compound containing a ribose moiety; preferably, nucleotide, nucleoside, analog thereof or monophosphate derivative thereof or polyphosphate derivative thereof; more preferably, ribonucleotide, deoxyribonucleotide, galidesvir, ribavirin, favipiravir-RTP, remdesivir or its triphosphate metabolite, cytidine 5′-monophosphate (5′-CMP); hydrogen peroxide;
oligopeptide or cyclopeptide;
buffer reagent; preferably, tris;
smaller molecular drug; preferably, nucleoside analogue medicines; more preferably, galidesvir, ribavirin, favipiravir-RTP, remdesivir or its triphosphate metabolite;
neurotransmitter; preferably, catecholamine or derivative thereof;
compound with a specific chirality; preferably, L-Norepinephrine or D-Norepinephrine;
analyte containing an isotope; preferably, catechol-D6 (deuterium replaces all the hydrogen atoms on catechol);
chemical intermediate;
or any combination thereof.

### BRIEF DESCRIPTION OF THE DRAWINGS

FIG. 1 shows conceptual demonstration of PNRSS. a. A PNRSS strand. A PNRSS strand is composed of functional modules as described. The reaction section, the most critical module, contains one or more reactive sites forming a fixed reactant. b. The measurement configuration. During PNRSS, an MspA nanopore serves to dock a streptavidin tethered PNRSS strand. The reaction section (dark yellow) is located precisely at the pore restriction for optimum performance. c. The design of PNRSS to probe single molecule reaction between Ni²⁺ and a dual guanine reactant. Two neighbouring guanines on the PNRSS strand cooperatively bind a Ni²⁺ ion. d. A continuous trace containing different states of a PNRSS measurement. Initially, the pore was unoccupied (i) and an open pore current was reported. A PNRSS strand was then captured by the pore, causing an immediate drop of the blockage level to Iₚ (ii). After the placement of Ni²⁺, reversible switching between I_{b} and Iₚ was observed, which respectively demonstrate the state when the PNRSS strand was not bound (iii) or bound (iv) with a Ni²⁺. e. Density scatter plot of ΔI vs. t_{off}. The local density around each point is colour coded. The histogram of ΔI with a Gaussian fitting is on the right of the scatter plot. Results of 13435 events are included. f. Concentration dependence. The reciprocal of interevent interval (1/τₒₙ) and the reciprocal of dwell time (1/τ_{off}) is plotted against [Ni²⁺]. The measurement was carried out as described in Methods. The PNRSS strand 13G/14G (Table 1) was applied. Nickel sulfate was added to trans at the desired final concentration.

FIG. 2 shows PNRSS with unnatural reactive components. a. The introduction of a 1,2,3-triazole (TAZ) to the PNRSS strand and its reaction mechanism. The PNRSS strand 14TAK (Table 1) was reacted with 3-azidopropylamine, as described in FIG. 17. The generated TAZ serves as the fixed reactant. Ni²⁺, the mobile reactant, becomes involved in reversible coordination with a TAZ. b. Trace demonstration. A continuous trace containing Ni²⁺ binding events. Characteristic noise fluctuations were consistently observed during Ni²⁺ binding. c. A representative event. A zoomed-in demonstration of a binding event. Noise fluctuations during Ni²⁺ binding indicate possible reactive intermediates observable by PNRSS. d. Density scatter plot of ΔI vs. τ_{off}. The local density around each point is colour coded. A highly uniform event population was observed. Results of 905 events are included. e. Concentration dependence. The reciprocal of inter-event interval (1/τₒₙ) and the reciprocal of dwell time (1/τ_{off}) is plotted against [Ni²⁺] (Table 6). The measurement was carried out as described in Methods. Nickel sulfate was added to trans at the desired final concentration.

FIG. 3 shows PNRSS with phenylboronic acid. a. Introduction of phenylboronic acid (PBA) and the corresponding reaction mechanism. A PBA was introduced with CuAAC as described in FIGS. 24-26. b-g. The PBA on 14PBA serves as the fixed reactant. Mobile reactants containing 1,2-diols or 1,3-diols, such as catechol (b), ethylene glycol (c), glycerol (d), L-lactic acid (e), vitamin C (f) or vitamin B6 (g), all report clear and distinct binding events. h. Resorcinol which lacks a compatible reactive group, fails to report any binding events. The experiments were carried out as described in Methods. Catechol (500 μM, b), ethylene glycol (18 mM, c), glycerol (12 mM, d), L-lactic acid (5 mM, e), vitamin C (1.6 mM, f), vitamin B6 (50 μM, g) or resorcinol (1 mM, h) were added to trans reaching the aforementioned final concentrations. Corresponding scatter plots of ΔI vs. τ_{off}, results of 1/τₒₙ and 1/τ_{off} against different analyte concentrations and time histograms are provided in FIGS. 27-38.

FIG. 4 shows repetitive PNRSS measurement of irreversible reactions. a. Introduction of PBA and the corresponding reaction mechanism. A PBA is introduced by CuAAC as described in FIGS. 24-26. The PBA on the PNRSS strand serves as the fixed reactant. Hydrogen peroxide, acting as a mobile reactant, may either reversibly bind to PBA or irreversibly oxidize the PBA to a phenol. b. A trace containing the reversible (i)-(ii) and irreversible (iii) reactions of a PBA. c. The PNRSS strategy to cope with irreversible reactions. Upon irreversible oxidation, the docked PNRSS strand is voltage ejected and reloaded to initiate a new measurement cycle. d. A trace containing repetitive PNRSS measurement. Four consecutive cycles of measurements are demonstrated. The star label marks the moment when the voltage ejection and reloading was performed (Video 3).

FIG. 5 shows PNRSS sensing of epinephrine, norepinephrine and isoprenaline. a. The reaction mechanism. Norepinephrine, epinephrine and isoprenaline all contain a 1,2-benzene diol moiety, capable of binding a PBA. b. Representative events of epinephrine, norepinephrine or isoprenaline binding. The events were low pass Butterworth filtered with a cut off frequency of 100 Hz (FIG. 50). All events appear negative going (I_{b}<Iₚ). c-d. A representative trace containing norepinephrine, epinephrine or isoprenaline binding events. The trace was Butterworth filter separated into the low pass (c) and the high pass (d) portion. The cut off frequency is 100 Hz (FIG. 50). e. A confusion matrix was generated based on 1455 events fed into a SVC model (FIG. 51). f. The scatter plot of low pass (Lp) and high pass (Hp) amplitude standard deviation (S.D.) from PNRSS sensing of a mixture of catecholamine events (FIG. 52). Results of 150 events are included. The decision boundary, which separates the scatter plot to green (norepinephrine), pink (epinephrine) and orange (isoprenaline) colour coded regions, was determined by a machine learning algorithm (FIG. 51).

FIG. 6 shows PNRSS sensing of remdesivir and remdesivir triphosphate metabolite. a. The sensing mechanism. Remdesivir and the remdesivir metabolite both contain a ribose moiety, capable of binding to a PBA. b. Representative events of remdesivir and remdesivir metabolite. The label R and M respectively marks remdesivir and remdesivir metabolite. The events were Butterworth low pass filtered with a cut off frequency of 100 Hz (FIG. 50). Both events appear positive going (I_{b}>Iₚ). Binding of remdesivir results in time extended events with characteristic noise fluctuations. Remdesivir metabolite on the other hand reports transient events with minimum noise. c. A scatter plot of event amplitude standard deviation (S.D.) against the event dwell time. The histogram of the event amplitude standard deviation (S.D.) and its Gaussian fitting results were plotted to the right. Results of 119 events are included. d. A scatter plot of high pass (Hp) and low pass (Lp) amplitude S.D. Both analytes are clearly separated in the scatter plot. Results of 126 events are included. e. A continuous trace containing binding events from remdesivir and remdesivir metabolite. The identity of each event is called based on event characteristics as described in d.

FIG. 7 shows Ni²⁺ binding to a dual guanine reactant. a. The schematic diagram. The PNRSS measurement was carried out as described in FIG. 1c. A buffer of 1.5 M KCl, 10 mM HEPES, pH 7.0. was used. A +180 mV potential was continuously applied. The PNRSS strand 13G/14G (Table 1) contains two neighbouring guanines, cooperatively serving as a ligand to bind Ni²⁺. Ni²⁺, which acts as a mobile reactant, was added to trans reaching a desired final concentration. b. Representative traces acquired with varying Ni²⁺ concentrations. The Ni²⁺ concentrations were adjusted between 0 and 1 mM and are marked on the left of each corresponding trace, which shows that the rate of event appearance increases when the Ni²⁺ concentration is raised.

FIG. 8 shows PNRSS with no fixed reactant. a. The schematic diagram. The measurements were carried out as described in Methods. A buffer of 1.5 M KCl, 10 mM HEPES, pH 7.0 was used. A +180 mV potential was continuously applied. The PNRSS strand 14X (Table 1), in which the reaction section is composed of five consecutive abasic sites, was applied. Ni²⁺ serves as the mobile reactant. In principle, the abasic sites are incapable of binding Ni²⁺. b. Representative traces from corresponding PNRSS measurements. Ni²⁺ was added to trans with a final concentration of 0-1 mM. The final concentrations of Ni²⁺ are marked on the left of each corresponding trace. No events of Ni²⁺ binding were observed, concluding that the PNRSS strand 14X fails to report any Ni²⁺ binding events.

FIG. 9 shows Ni²⁺ binding with a dual guanine reactant (simulation). The optimized structures of Ni²⁺ binding with a dual guanine ligand. (dGMP)₂-Ni-4 wt with (a) the low-spin state, (c) the high-spin state, and (dGMP)₂-Ni-5 wt with (b) the low-spin state, (d) the high-spin state are shown. Water molecules which participate in the binding are also shown. Green, grey, blue, red, orange and white balls represent Ni, C, N, O, P, and H atoms respectively.

FIG. 10 shows PNRSS measurement and data analysis. a. A representative trace of PNRSS measurement. The cartoon describes different states of the pore during the measurement. State (i) represents an unoccupied pore, at which the measured current is the open pore current (I₀). State (ii) and (iii) represents a pore occupied with a PNRSS strand, in which the fixed reactant of the PNRSS strand is either not bound (ii) or bound (iii) with a mobile reactant. The measured current at state (ii) or (iii) is respectively defined as Iₚ or I_{b}. b. A zoomed-in view of the trace containing binding events. Consecutive binding events which appear as resistive pulses (grey) are clearly observed. The event amplitude (ΔI) is defined as Δ=I_{b}−Iₚ. Binding of a mobile reactant may generate either positive (I_{b}>Iₚ) or negative going (I_{b}<Iₚ) events, resulting in positive or negative ΔI values. The inter-event interval (tₒₙ) and the event dwell time (t_{off}) are defined as described on the trace. c-d. The derivation of mean inter-event interval (τₒₙ) (c) and the mean event dwell time (τ_{off}) (d). tₒₙ and t_{off} are fit respectively with a single exponential function y=a*xp (−x/τ), from which the mean inter-event interval (τₒₙ) and the mean event dwell time (τ_{off}) are derived. e. The derivation of mean event amplitude (ΔI). ΔI is derived from the central position of the Gaussian fitting. The demonstrative results are from measurements as described in FIG. 1, carried out with the PNRSS strand 13G/14G (Table 1) and Ni²⁺. Unless otherwise stated, analysis of all PNRSS measurements in this paper was carried out following the described definition.

FIG. 11 shows τₒₙ and τ_{off} of Ni²⁺ binding to a dual guanine reactant. Histograms of the inter-event interval (tₒₙ) and the event dwell time (t_{off}) acquired with different Ni²⁺ concentrations were presented. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 13G/14G (Table 1) was applied. A buffer of 1.5 M KCl, 10 mM HEPES, pH 7.0 was used. A +180 mV potential was continuously applied.

FIG. 12 shows binding of other metal ions to a dual guanine reactant. a. The schematic diagram. The PNRSS measurements were similar to that described in FIG. 1c. A buffer of 1.5 M KCl, 10 mM HEPES, pH 7.0. was used. A +180 mV potential was continuously applied. Two neighbouring guanines on the PNRSS strand (13G/14G) serve as the fixed reactant (Table 1). Divalent ions such as Zn²⁺, Cd²⁺, Co²⁺ or Cu²⁺ serve as the mobile reactant. b-e. Representative traces of PNRSS measurements when Zn²⁺ (b), Cd²⁺ (c), Co²⁺ (d) or Cu²⁺ (e) was placed in trans. The final concentration of the added divalent ions is noted on the left of each corresponding trace. According to the results, Zn²⁺ (b) or Cd²⁺ (c) reports no binding to the PNRSS strand. However, Co²⁺ (d) or Cu²⁺ (e) show noticeable binding events. The above results demonstrate that binding of Co²⁺ or Cu²⁺ to a dual guanine ligand is also observable by PNRSS⁹. Further investigations will be carried out in a separate, follow up study.

FIG. 13 shows PNRSS measurement with a sole adenine reactant. a. The schematic diagram. The measurements were carried out as described in Methods. A buffer of 1.5 M KCl, 10 mM HEPES, pH 7.0. was used. A +180 mV potential was continuously applied. The PNRSS strand 14A (Table 1) was applied. 14A contains a sole adenine, serving as the fixed reactant. Ni²⁺ serves as the mobile reactant. b. Representative traces when Ni²⁺ was added to trans with varying final concentrations. The concentration of Ni²⁺ is noted on the left of each trace. Binding of Ni²⁺ to a sole adenine results in spiky, negative going events. c. A plot of 1/τₒₙ or 1/τ_{off} vs. the Ni²⁺ concentration. 1/τₒₙ is linearly correlated to the final concentration of Ni²⁺. However, 1/τ_{off} stays constant. d. An event scatter plot of ΔI vs. τ_{off}. The Ni²⁺ concentration was 0.8 mM. All events were extracted from a 15 min continuously recorded trace. 1137 events are included in the scatter plot. Each point is color coded according to the local point density. A major population measuring ˜39 pA in ΔI and a minor population measuring ˜56 pA in ΔI were respectively identified. The event histogram of ΔI is attached to the right margin of the scatter plot. The two populations of ΔI are respectively Gaussian fitted and superimposed on the histogram. The above observation demonstrates that coordination interaction between Ni²⁺ and a sole adenine is observable by PNRSS. The different choice of the fixed reactant results in different binding kinetics. From a previous study, an adenine has two possible binding sites to bind a Ni²⁺, which may serve to explain the two populations of events that were observed⁹².

FIG. 14 shows τₒₙ and τ_{off} of Ni²⁺ binding to a sole adenine reactant. Histograms of the inter-event interval (τₒₙ) and the event dwell time (τ_{off}) with different Ni²⁺ concentrations are presented. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14A (Table 1) was applied. A buffer of 1.5 M KCl, 10 mM HEPES, pH 7.0 was used. A +180 mV potential was continuously applied.

FIG. 15 shows PNRSS measurement with a sole guanine reactant. a. The schematic diagram. The measurements were carried out as described in Methods. A buffer of 1.5 M KCl, 10 mM HEPES, pH 7.0. was used. A +180 mV potential was continuously applied. The PNRSS strand 14G (Table 1) was applied. 14G contains a sole guanine, serving as the fixed reactant. Ni²⁺ serves as the mobile reactant. b. Representative traces when Ni²⁺ was added to trans with varying final concentrations. The concentrations of Ni²⁺ are noted on the left of each trace. Binding events of Ni²⁺ were observed as spiky, negative going events. c. A plot of 1/τₒₙ or 1/τ_{off} vs. the Ni²⁺ concentration. 1/τₒₙ is linearly correlated to the final concentration of Ni²⁺. However, 1/τ_{off} stays constant. d. An event scatter plot of ΔI vs. τ_{off}. The Ni²⁺ concentration was 0.8 mM. All events were extracted from a 15 min continuously recorded trace. 1773 events are included in the scatter plot. Each point is color coded according to the local point density. A single population of events, measuring ˜30 pA in ΔI was identified. The event histogram of ΔI, which is superimposed with its Gaussian fitting result, is attached to the right margin of the scatter plot. The above observation demonstrates that coordination interaction between Ni²⁺ and a sole guanine is observable by PNRSS. The different choice of the fixed reactant results in different binding kinetics⁹².

FIG. 16 shows τₒₙ and τ_{off}, of Ni²⁺ binding to a sole guanine reactant. Histograms of the inter-event interval (τₒₙ) and the event dwell time (τ_{off}) with different Ni²⁺ concentrations are presented. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14G (Table 1) was applied. A buffer of 1.5 M KCl, 10 mM HEPES, pH 7.0 was used. A +180 mV potential was continuously applied.

FIG. 17 shows chemical synthesis of the PNRSS strand 14TAZ. a. The cartoon diagram of TAZ introduction to a PNRSS strand. b. The CuAAC reaction. To produce a triazole on a PNRSS strand, 3-azidopropylamine was reacted with 14TAK (Table 1) by Huisgen copper (I)-catalyzed azide-alkyne 1,3-dipolarcycloaddition (CuAAC)⁹³. Briefly, 10 L solution of DNA 14TAK (100 μM), 6 μL acetonitrile solution of 3-azidopropylamine (330 mM), 1.5 μL copper sulfate (20 mM), 3 μL sodium ascorbate (20 mM) and 3.5 μL Milli-Q water were added to a 6 μL HEPES buffer (100 mM HEPES, pH 7.4) and shaken at 600 rpm at 25° C. for 4 h. Afterwards, 6 μL EDTA solution (100 mM) was added to the mixture to terminate the reaction. The product DNA was purified using Micro Bio-Spin 6 Columns (Bio-Rad). To confirm the success of conjugation, the purified product was analysed by liquid chromatography-mass spectrometry (Xevo G2-XS QTOF MS+ Acuqity UPLC I-Class plus, Waters Corporation) equipped with an electrospray ionization (ESI) source. The product DNA is referred to as 14TAZ (Table 1) and used directly in downstream PNRSS measurements. c. Mass spectrometry results of 14TAK. Calculated: 18271.0, found: 18272.1. d. Mass spectrometry results of 14TAZ. Calculated: 18371.1, found: 18372.3.

FIG. 18 shows single molecule characterization of the PNRSS strand 14TAZ. a. The generation of a triazole and its Ni²⁺ sensing mechanism. The PNRSS strand 14TAK (Table 1) contains a sole alkyne (blue arc). 3-azidopropylamine (grey symbol) was reacted with 14TAK by CuAAC (FIG. 17), resulting in the production of 14TAZ, which contains a sole triazole as the fixed reactant (blue+grey). Ni²⁺, serving as the mobile reactant, is reversibly coordinated with a triazole⁹⁴. b-c. Iₚ measurement with 14TAK (b) or 14TAZ (c). A +180 mV potential was applied. A clear difference of Iₚ was observed when a 14TAK (b) or a 14TAZ (c) was captured by the pore, providing a single molecule evidence for the success of TAZ generation. This difference is also summarized in the event histogram of 14TAK (d) or a 14TAZ (f), when static pore blockage measurements were performed. The mean blockage amplitude (I̅ₚ̅) values were summarized in Table 5. e, g. PNRSS was carried out with either 14TAK (e) or 14TAZ (g). Ni²⁺ was applied as the mobile reactant and added to trans with a 1 mM final concentration. No Ni²⁺ binding events were observed with 14TAK (e). However, characteristic bindings were observed with 14TAZ (g), again confirming that a TAZ has been generated on the strand.

FIG. 19 shows Ni²⁺ binding to a triazole. a. The schematic diagram. The PNRSS measurement was carried out as described in FIG. 2. The PNRSS strand 14TAZ (Table 1), which contains a sole triazole, serves as the fixed reactant. Ni²⁺, which forms reversible coordination with the triazole, serves as the mobile reactant. b. Representative traces acquired with varying Ni²⁺ concentrations. The Ni²⁺ concentrations were adjusted between 0 and 1 mM and respectively noted on the left of each corresponding trace. Ni²⁺ binding results in negative going events (I_{b}<Iₚ). Characteristic noises were produced when Ni²⁺ was bound to the triazole, reporting the occurrence of this specific reaction unambiguously. The rate of event appearance increases when the Ni²⁺ concentration is raised, providing concrete evidence that the observed events result from Ni²⁺ binding.

FIG. 20 shows τₒₙ and τ_{off} of Ni²⁺ binding to a triazole. Histograms of the inter-event interval (τₒₙ) and the event dwell time (τ_{off}) with different Ni²⁺ concentrations are presented. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14TAZ (Table 1) was applied. A buffer of 1.5 M KCl, 10 mM HEPES, pH 7.0 was used. A +180 mV potential was continuously applied.

FIG. 21 shows Co²⁺ binding to a triazole. a. The schematic diagram. The measurements were carried out as described in Methods. A buffer of 1.5 M KCl, 10 mM HEPES, pH 7.0. was used. A +180 mV potential was continuously applied. The PNRSS strand 14TAZ (Table 1) was applied. 14TAZ contains a sole triazole, serving as the fixed reactant. Co²⁺ serves as the mobile reactant. b. Representative traces when Co²⁺ was added to trans. The concentrations of Co²⁺ were adjusted between 0-250 μM and respectively marked on the left of each corresponding trace. Binding events of Co²⁺ were observed as spiky, negative going events. c. A plot of 1/τₒₙ or 1/τ_{off} vs. the Co²⁺ concentration. 1/τₒₙ is linearly correlated to the final concentration of Co²⁺. However, 1/τ_{off} stays constant. d. An event scatter plot of ΔI vs. t_{off}. The Co²⁺ concentration was 0.2 mM. All events were extracted from a 15 min continuously recorded trace. 3632 events are included in the scatter plot. Each point is color coded according to the local point density. A single population of events, measuring ˜−25 pA in ΔI was identified. The event histogram of ΔI is attached to the right margin of the scatter plot. The event histogram of ΔI, which is superimposed with its Gaussian fitting result, is attached to the right margin of the scatter plot. The above observation demonstrates that coordination interaction between Co²⁺ and a sole triazole is observable by PNRSS. The binding characteristics is however different from that of Ni²⁺

FIG. 22 shows τₒₙ and τ_{off} of Co²⁺ binding to a triazole. Histograms of the inter-event interval (τₒₙ) and the event dwell time (τ_{off}) with different Co²⁺ concentrations are presented. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14TAZ (Table 1) was applied. A buffer of 1.5 M KCl, 10 mM HEPES, pH 7.0 was used. A +180 mV potential was continuously applied.

FIG. 23 shows sequential addition of Co²⁺ and Ni²⁺ when measured by 14TAZ. The measurements were carried out as described in Methods. A buffer of 1.5 M KCl, 10 mM HEPES, pH 7.0. was used. A +180 mV potential was continuously applied. The PNRSS strand 14TAZ (Table 1) was applied. a. A representative PNRSS trace when no mobile reactant was added. b. A representative PNRSS trace when Co²⁺ was added at a 250 μM final concentration. Only Co²⁺ binding events were observed. c. A representative PNRSS trace when Ni²⁺ was further added with a 400 μM final concentration. Both Co²⁺ and Ni²⁺ binding events were observed. The unique event noises generated by Ni²⁺ provide unambiguous evidence for event recognition.

FIG. 24 shows ¹H NMR spectrum of 4-(Azidomethyl) benzeneboronic acid. ¹H NMR (BRUKER AVANCE III, 400 MHz, 298K, DMSO-d6) δ 8.08 (s, 2H), 7.81 (d, J=8.0 Hz, 2H), 7.33 (d, J=8.0 Hz, 2H), 4.44 (s, 2H).⁹⁰

FIG. 25 shows chemical synthesis of the PNRSS strand 14PBA. a. The cartoon diagram of PBA introduction. b. The reaction. To produce a PBA on a PNRSS strand, 4-(azidomethyl) benzeneboronic acid was reacted with 14TAK (Table 1) by Huisgen copper (I)-catalyzed azide-alkyne 1,3-dipolarcycloaddition (CuAAC)⁹⁵. Briefly, 10 L solution of DNA 14TAK (100 μM), 6 μL 4-(azidomethyl) benzeneboronic acid (dissolved in MeCN, 200 mM), 1.5 μL copper sulfate (20 mM), 3 μL sodium ascorbate (20 mM) and 3.5 L Milli-Q water were added to a 6 μL HEPES buffer (100 mM HEPES, pH 7.4) and shaken at 600 rpm at 25° C. for 4 h. Subsequently, 6 μL EDTA solution (100 mM) was added to the mixture to terminate the reaction. The product DNA was purified using Micro Bio-Spin 6 Columns (Bio-Rad). To confirm the success of conjugation, the purified product was analysed by liquid chromatography-mass spectrometry (Xevo G2-XS QTOF MS+ Acuqity UPLC I-Class plus, Waters Corporation) equipped with an electrospray ionization (ESI) source. This functionalized DNA is referred to as 14PBA (Table 1) and used directly in downstream PNRSS measurements. c. Mass spectrometry results of 14PBA. For [14PBA], the calculated mass: 18448.0. For [14PBA-2H₂O], the calculated mass: 18412.0, found: 18413.1. During mass spectrometry measurement, every boronic acid loses 2H₂O, likely resulted from a strong intramolecular interaction of boronic acid with the DNA phosphate backbone, a phenomenon also reported in literature⁹⁶.

FIG. 26 shows single molecule characterization of the PNRSS strand 14PBA. a. Introduction of phenylboronic acid (PBA) and its catechol sensing mechanism. The PNRSS strand 14TAK (Table 1) contains a sole alkyne (blue arc). 4-(azidomethyl) benzeneboronic acid (grey symbol) was reacted with 14TAK by CuAAC, resulting in the production of 14PBA, which contains a sole PBA as the fixed reactant. Details of synthesis and characterization of 14PBA are provided in FIGS. 24-25. Catechol, which forms reversible interactions with a PBA, is applied as the mobile reactant. b-c. The Iₚ measurements with 14TAK (b) or 14PBA (c). A +160 mV potential was applied. An obvious difference of Iₚ was observed when a 14TAK (b) or a 14PBA (c) was captured by the pore, providing a single molecule evidence for the success of PBA conjugation. This difference is also summarized in the event histogram of Iₚ when 14TAK (d) or 14PBA (f) was measured during static pore blockage measurements. The mean blockage amplitude (Iₚ) values are summarized in Table 7. e, g. PNRSS was carried out with either 14TAK (e) or 14PBA (g). Catechol was applied as the mobile reactant and added to trans with a 500 μM final concentration. No catechol binding events were observed with 14TAK (e). However, characteristic bindings were observed with 14PBA (g), again confirming that a PBA has been successfully conjugated to the strand.

FIG. 27 shows catechol binding to a PBA. a. The schematic diagram. The PNRSS strand 14PBA (Table 1) contains a sole PBA at site 14, capable of binding catechol 97, as illustrated by the cartoon diagram. b. Representative traces containing catechol binding events. A buffer of 1.5 M KCl, 10 mM HEPES, pH 8.0 was used. A +160 mV potential was continuously applied. Catechol was added to trans with a final concentration of 0-0.5 mM, marked on the left of each corresponding trace. Binding of catechol results in positive going events (I_{b}>Iₚ). The rate of event appearance is increases when the catechol concentration is raised. c. Concentration dependence. The reciprocal of inter-event interval 1/τₒₙ and the reciprocal of dwell time 1/τ_{off} is plotted against the final concentration of catechol in trans. 1/τₒₙ shows a linear correlation with the concentration of catechol. 1/τ_{off} stays constant. d. Scatter plot of ΔI vs. τ_{off}. 100 events are included in the scatter plot. The histogram of ΔI, superimposed with its Gaussian fitting result, is plotted to the right of the scatter plot. The catechol concentration was 400 μM. The events were extracted from a 15 min continuously recorded trace.

FIG. 28 shows τₒₙ and τ_{off} of catechol binding to a PBA. Histograms of the inter-event interval (tₒₙ) and the event dwell time (t_{off}) with different catechol concentrations are presented. Catechol was added to trans with a final concentration of 0.1-0.5 mM. The applied concentration is marked on the left of each corresponding Histogram. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 29 shows ethylene glycol binding to a PBA. a. The schematic diagram. The PNRSS strand 14PBA (Table 1) contains a sole PBA at site 14, capable of binding ethylene glycol⁹⁸, as illustrated by the cartoon diagram. b. Representative traces containing ethylene glycol binding events. A buffer of 1.5 M KCl, 10 mM HEPES, pH 8.0 was used. A +160 mV potential was continuously applied. Ethylene glycol was added to trans with a final concentration of 0-18 mM, marked on the left of each corresponding trace. The rate of event appearance increases when the ethylene glycol concentration is raised. c. Concentration dependence. The reciprocal of inter-event interval (1/τₒₙ) and the reciprocal of dwell time (1/τ_{off}) is plotted against the final concentration of ethylene glycol. 1/τₒₙ demonstrates a linear correlation with the concentration of ethylene glycol. 1/τ_{off} remains constant. d. Scatter plot of ΔI vs. t_{off}. 135 events are included in the scatter plot. The histogram of ΔI, superimposed with its Gaussian fitting result, is plotted to the right of the scatter plot. The ethylene glycol concentration was 14 mM. The events were extracted from a 15 min continuously recorded trace.

FIG. 30 shows τₒₙ and τ_{off} of ethylene glycol binding to a PBA. Histograms of the inter-event interval (tₒₙ) and the event dwell time (t_{off}) with different ethylene glycol concentrations are presented. Ethylene glycol was added to trans with a final concentration of 2-18 mM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 31 shows glycerol binding to a PBA. a. The schematic diagram. The PNRSS strand 14PBA (Table 1) contains a sole PBA at site 14, capable of binding glycerol⁹⁸, as illustrated by the cartoon diagram. b. Representative traces containing glycerol binding events. A buffer of 1.5 M KCl, 10 mM HEPES, pH 8.0 was used. A +160 mV potential was continuously applied. Glycerol was added to trans with a final concentration of 0-12 mM, marked on the left of each corresponding trace. The rate of event appearance increases when the glycerol concentration is raised. c. Concentration dependence. The reciprocal of inter-event interval (1/τₒₙ) and the reciprocal of dwell time (1/τ_{off}) is plotted against the final concentration of glycerol. 1/τₒₙ demonstrates a linear correlation with the concentration of glycerol. 1/τ_{off} remains constant. d. Scatter plot of ΔI vs. τ_{off}. 267 events are included in the scatter plot. The histogram of ΔI, superimposed with its Gaussian fitting result, is plotted to the right of the scatter plot. The glycerol concentration was 10 mM. The events were extracted from a 15 min continuously recorded trace.

FIG. 32 shows τₒₙ and τ_{off} of glycerol binding to a PBA. Histograms of the inter-event interval (tₒₙ) and the event dwell time (t_{off}) with different glycerol concentrations are presented. Glycerol was added to trans with a final concentration of 4-12 mM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 33 shows L-lactic acid binding to a phenylboronic acid (PBA) reactant. a. The schematic diagram. The PNRSS strand 14PBA (Table 1) contains a sole PBA at site 14, capable of binding L-lactic acid⁹⁷, as illustrated by the cartoon diagram. b. Representative traces containing L-lactic acid binding events. A buffer of 1.5 M KCl, 10 mM HEPES, pH 8.0 was used. A +160 mV potential was continuously applied. L-lactic acid was added to trans with a final concentration of 0-5 mM, marked on the left of each corresponding trace. The rate of event appearance increases when the L-lactic concentration is raised. c. Concentration dependence. The reciprocal of inter-event interval (1/τₒₙ) and the reciprocal of dwell time (1/τ_{off}) is plotted against the final concentration of L-lactic acid. 1/τₒₙ demonstrates a linear correlation with the concentration of L-lactic acid. 1/τ_{off} stays constant. d. Scatter plot of ΔI vs. t_{off}. 213 events are included in the scatter plot. The histogram of ΔI, superimposed with its Gaussian fitting result, is plotted to the right of the scatter plot. The lactic acid concentration was 4 mM. The events were extracted from a 15 min continuously recorded trace.

FIG. 34 shows τₒₙ and τ_{off} of L-lactic acid binding to a PBA. Histograms of the inter-event interval (tₒₙ) and the event dwell time (t_{off}) with different L-lactic acid concentrations are presented. L-lactic acid was added to trans with a final concentration of 1-5 mM. The applied concentration is marked on the left of each corresponding histogram plot. All histograms were respectively fit with a single exponential function y=a*exp(−x/τ), from which the mean inter-event interval (τₒₙ) and the mean event dwell time (rot) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 35 shows vitamin C binding to a PBA. a. The schematic diagram. The PNRSS strand 14PBA (Table 1) contains a sole PBA at site 14, capable of binding vitamin C⁹⁹, as illustrated by the cartoon diagram. b. Representative traces containing vitamin C binding events. A buffer of 1.5 M KCl, 10 mM HEPES, pH 8.0 was used. A +160 mV potential was continuously applied. vitamin C was added to trans with a final concentration of 0-2 mM, marked on the left of each corresponding trace. The rate of event appearance increases when the vitamin C concentration is raised. c. Concentration dependence. The reciprocal of inter-event interval (1/τₒₙ) and the reciprocal of dwell time (1/τ_{off}) is plotted against the final concentration of vitamin C. 1/τₒₙ demonstrates a linear correlation with the concentration of vitamin C. 1/1τ_{off} stays constant. d. Scatter plot of ΔI vs. τ_{off}. 120 events are included in the scatter plot. The histogram of ΔI, superimposed with its Gaussian fitting result, is plotted to the right of the scatter plot. The vitamin C concentration was 1.6 mM. The events were extracted from a 15 min continuously recorded trace.

FIG. 36 shows τₒₙ and τ_{off} of vitamin C binding to a PBA. Histograms of the inter-event interval (tₒₙ) and the event dwell time (t_{off}) with different vitamin C concentrations are presented. Vitamin C was added to trans with a final concentration of 0.4-2.0 mM. The applied concentration is marked on the left of each corresponding histogram. All histograms were respectively fit with a single exponential function y=a*exp(−x/τ), from which the mean inter-event interval (τₒₙ) and the mean event dwell time (τ_{off}) were derived and marked in each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 37 shows vitamin B6 binding to a PBA. a. The schematic diagram. The PNRSS strand 14PBA (Table 1) contains a sole PBA at site 14, capable of binding vitamin B6¹⁰⁰, as illustrated by the cartoon diagram. b. Representative traces containing vitamin B6 binding events. A buffer of 1.5 M KCl, 10 mM HEPES, pH 8.0 was used. A +160 mV potential was continuously applied. Vitamin B6 was added to trans with a final concentration of 0-0.05 mM, marked on the left of each corresponding trace. The rate of event appearance increases when the vitamin B6 concentration is raised. c. Concentration dependence. The reciprocal of inter-event interval (1/τₒₙ) and the reciprocal of dwell time (1/τ_{off}) is plotted against the final concentration of vitamin B6. (1/τₒₙ) demonstrates a linear correlation with the concentration of vitamin B6. (1/τ_{off}) stays constant. d. Scatter plot of ΔI vs. t_{off}. 763 events are included in the scatter plot. The histogram of ΔI, superimposed with its Gaussian fitting result, is plotted to the right of the scatter plot. The vitamin B6 concentration was 40 μM. The events were extracted from a 15 min continuously recorded trace.

FIG. 38 shows τₒₙ and τ_{off} of vitamin B6 binding to a PBA. Histograms of the inter-event interval (tₒₙ) and the event dwell time (t_{off}) with different vitamin B6 concentrations are presented. Vitamin B6 was added to trans with a final concentration of 0.01-0.05 mM. The applied concentration is marked on the left of corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 39 shows Tris binding to a PBA. a. The schematic diagram. The PNRSS strand 14PBA (Table 1) contains a sole PBA at site 14, capable of binding tris, as illustrated by the cartoon diagram. b. Representative traces containing tris binding events. A buffer of 1.5 M KCl, 10 mM HEPES, pH 8.0 was used. A +160 mV potential was continuously applied. Tris was added to trans with a final concentration of 0-1.0 mM, marked on the left of each corresponding trace. The rate of event appearance is increased when the tris concentration is raised. c. Concentration dependence. The reciprocal of inter-event interval (1/τₒₙ) and the reciprocal of dwell time (1/τ_{off}) is plotted against the final concentration of tris. (1/τₒₙ) demonstrates a linear correlation with the concentration of tris. (1/τ_{off}) remains constant. The events were extracted from a 15 min continuously recorded trace for each condition.

FIG. 40 shows τₒₙ and τ_{off} of tris binding to a PBA. Histograms of the inter-event interval (tₒₙ) and the event dwell time (t_{off}) with different tris concentrations are presented. Tris was added to trans with a final concentration of 0.2-1.0 mM. The applied concentration was marked on the left of corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 41 shows observing chemical intermediates with PNRSS. a. A suggested reaction model of tris, when bound with a PBA. Tris could be protonated or deprotonated depending on the environment pH. b. Representative PNRSS events containing chemical intermediates, acquired at pH 8.0. Transition between state 0, 1 or 2 were observed. Transition to other states was however never observed. c-d. Representative traces acquired at pH 7.0 (c) or at pH 8.0 (d). At pH 7.0, tris binding to a PBA results in only one type of blockage level I_{b1}. However, at pH 8.0, tris binding results in a new blockage level I_{b2}, on top of I_{b1}. e-f. Event scatter plots of ΔI vs. t_{off} formed from events acquired at pH 7.0 (e) or pH 8.0 (f). In the scatter plot, a new event population of I_{b2} in ΔI is observed at pH 8.0. All above measurements were carried out as described in Methods. 1577 events are included in e. 8261 events are included in f. Measurement at a higher pH results in a much higher rate of event appearance. 14PBA was applied as the PNRSS strand. All above measurements were performed with a buffer of 1.5 M KCl, 10 mM tris. A +160 mV potential was continuously applied. The scatter plots (e, f) were formed from continuous 15 min recordings for each condition.

FIG. 42 shows investigation of the chemical nature of irreversible oxidation of a PBA. The PNRSS measurement was carried out similarly to that described in FIG. 4. The electrolyte buffer was 1.5 M KCl, 10 mM HEPES, pH 8.0. The PNRSS strand 14 PBA was added to cis with a 10 nM final concentration. H₂O₂, Ni²⁺ and glycerol were simultaneously added to trans with a 5.4 mM, a 0.2 mM and a 8 mM final concentration respectively. A +160 mV potential was continuously applied. a. A representative trace acquired during PNRSS. Binding of H₂O₂ or glycerol to a PBA results in positive going events, whereas binding of Ni²⁺ results in negative going events. The PBA may as well be irreversibly oxidized by H₂O₂ to generate a phenol (red arrow marked). Afterwards, binding of H₂O₂ or glycerol are no longer observed from the trace. b. A zoomed-in view of a trace segment from a (blue marked). Binding of H₂O₂ (green triangle), glycerol (orange circle) and Ni²⁺ (Purple square) are labelled respectively on the trace. c. The proposed mechanism of the binding. d. A zoomed-in view of a trace segment from a (grey marked). Only Ni²⁺ bindings (purple square) are still observable. e. The proposed mechanism. The disappearance of H₂O₂ and glycerol binding event and the retaining of Ni²⁺ binding have confirmed the hypothesis that the PBA has been irreversibly oxidized to a phenol.

FIG. 43 shows norepinephrine binding to a PBA. a. The schematic diagram. The PNRSS strand 14PBA (Table 1) contains a sole PBA at site 14, capable of binding norepinephrine, as illustrated by the cartoon diagram. b. Representative traces containing norepinephrine binding events. PNRSS measurements were carried out as described in FIG. 5. The electrolyte buffer was 1.5 M KCl, 10 mM HEPES, pH 8.0. A +160 mV potential was continuously applied. Norepinephrine was added to trans with a final concentration of 0-180 μM, marked on the left of each corresponding trace. The rate of event appearance increases when the norepinephrine concentration is raised. c. The reactive mechanism¹⁰¹. d. Concentration dependence. The norepinephrine concentration was modulated between 20-180 μM. 15 min continuous recording was performed for each condition. τₒₙ and τ_{off} values were derived as described in FIG. 10. Mean and standard deviation values were from three independent measurements for each condition. The reciprocal of inter-event interval (1/τₒₙ) and the reciprocal of dwell time (1/τ_{off}) is plotted against the final concentration of norepinephrine. (1/τₒₙ) demonstrates a linear correlation with the concentration of norepinephrine. (1/τ_{off}) stays constant. e. Scatter plot of ΔI vs. τ_{off}. 106 events are included in the scatter plot. The histogram of ΔI, superimposed with its Gaussian fitting result, is plotted to the right of the scatter plot. The norepinephrine concentration was 140 μM. The events were extracted from a 15 min continuously recorded trace.

FIG. 44 shows τₒₙ and τ_{off} of norepinephrine binding to a PBA. Histograms of the inter-event interval (tₒₙ) and the event dwell time (t_{off}) with different norepinephrine concentrations are presented. Norepinephrine was added to trans with a final concentration of 20-180 μM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 45 shows epinephrine binding to a PBA. a. The schematic diagram. The PNRSS strand 14PBA (Table 1) contains a sole PBA at site 14, capable of binding epinephrine, as illustrated by the cartoon diagram. b. Representative traces containing epinephrine binding events. PNRSS measurements were carried out as described in FIG. 5. The electrolyte buffer was 1.5 M KCl, 10 mM HEPES, pH 8.0. A +160 mV potential was continuously applied. Epinephrine was added to trans with a final concentration of 0-180 μM, marked on the left of each corresponding trace. The rate of event appearance increases when the epinephrine concentration is raised. c. The reactive mechanism¹⁰¹. d. Concentration dependence. The epinephrine concentration was modulated between 20-180 μM. 15 min continuously recording was performed for each condition. τₒₙ and τ_{off} values were derived as described in FIG. 10. Mean and standard deviation values were from three independent measurements for each condition. The reciprocal of inter-event interval (1/τₒₙ) and the reciprocal of dwell time (1/τ_{off}) are plotted against the final concentration of epinephrine. (1/τₒₙ) demonstrates a linear correlation with the concentration of epinephrine. (1/τ_{off}) stays constant. e. Scatter plot of ΔI vs. τ_{off}. The scatter plot was generated from a 15 min continuously recorded trace. 109 events are included in the scatter plot. The histogram of ΔI, superimposed with its Gaussian fitting result, is plotted to the right of the scatter plot. The epinephrine concentration was 140 μM. The events were extracted from a 15 min continuously recorded trace.

FIG. 46 shows τₒₙ and τ_{off} of epinephrine binding to a PBA. Histograms of the inter-event interval (tₒₙ) and the event dwell time (t_{off}) with different epinephrine concentrations are presented. Epinephrine was added to trans with a final concentration of 20-180 μM. The applied concentration was marked on the left of each corresponding histogram plot. All histograms were respectively fit with a single exponential function y=a*exp (−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 47 shows isoprenaline binding to a PBA. a. The schematic diagram. The PNRSS strand 14PBA (Table 1) contains a sole PBA at site 14, capable of binding isoprenaline, as illustrated by the cartoon diagram. b. Representative traces containing isoprenaline binding events. PNRSS measurements were carried out as described in FIG. 5. The electrolyte buffer was 1.5 M KCl, 10 mM HEPES, pH 8.0. A +160 mV potential was continuously applied. Isoprenaline was added to trans with a final concentration of 0-180 μM, marked on the left of each corresponding trace. The rate of event appearance increases when the isoprenaline concentration is raised. c. The reactive mechanism¹⁰¹. d. Concentration dependence. The isoprenaline concentration was modulated between 20-180 μM. 15 min continuous recording was performed for each condition. τₒₙ and τ_{off} values were derived as described in FIG. 10. Mean and standard deviation values were from three independent measurements for each condition. The reciprocal of inter-event interval (1/τₒₙ) and the reciprocal of dwell time (1/τ_{off}) are plotted against the final concentration of isoprenaline. (1/τₒₙ) demonstrates a linear correlation with the concentration of isoprenaline. (1/τ_{off}) stays constant. e. Event scatter plot of ΔI vs. t_{off}. The scatter plot was generated from a 15 min continuously recorded trace. 101 events are included in the scatter plot. The histogram of ΔI, superimposed with its Gaussian fitting result, is plotted to the right of the scatter plot. The isoprenaline concentration was 140 μM. The events were extracted from a 15 min continuously recorded trace.

FIG. 48 shows τₒₙ and τ_{off} of isoprenaline binding to a PBA. Histograms of the inter-event interval (tₒₙ) and the event dwell time (t_{off}) with different isoprenaline concentrations are presented. Isoprenaline was added to trans with a final concentration of 20-180 μM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 49 shows positive and negative going events. a. The schematic diagram. The PNRSS strand 14PBA (Table 1) contains a sole PBA at site 14, capable of binding catechol or norepinephrine, as illustrated by the cartoon diagram. b. A representative trace containing catechol and norepinephrine binding events. Catechol and norepinephrine were simultaneously added to trans, respectively reaching a 400 μM and a 140 μM final concentration respectively. Binding of a catechol or a norepinephrine to a PBA respectively reports positive (I_{b,C}, I_{b,C}>Iₚ) or negative going events (I_{b,N}, I_{b,N}<Iₚ). A visualized demonstration is also provided (Video 4). c. A zoomed-in demonstration of different binding events. Representative events of binding from a catechol (top left) or a norepinephrine (top right) are demonstrated. The chemical structures of a catechol (bottom left) or a norepinephrine (bottom right) when bound to a PBA are also demonstrated. d. Scatter plot of ΔI vs. t_{off}. The events were extracted from a 15 min continuously recorded trace. A total of 119 events are included in the scatter plot. From the scatter plot, binding events from catechol and norepinephrine result in two clearly separated populations. The histogram of ΔI is plotted to the right of the scatter plot. Two peaks of ΔI were respective Gaussian fitted and superimposed on the histogram.

FIG. 50 shows frequency split demonstration. When probed by PNRSS, binding of catecholamines, such as norepinephrine, epinephrine and isoprenaline, to a PBA results in rich information of chemical process, appearing as fluctuations in different frequency domains. The chemical structure of boronate ester complexes resulted from (a) norepinephrine, (e) epinephrine or (i) isoprenaline binding to a PBA are illustrated. During PNRSS, the raw trace was acquired with a 25 kHz sampling rate and low pass filtered at 1 kHz. The recorded traces were frequency split into low pass and the high pass portions, performed by Butterworth filtering. A cut-off frequency of 100 Hz and a filter order of 2 were selected. The raw events resulted from (b) norepinephrine, (f) epinephrine or (j) isoprenaline binding to a PBA were demonstrated. The low pass portion of the event resulted from (c) norepinephrine, (g) epinephrine or (k) isoprenaline binding to a PBA were demonstrated. The high pass portion of the event resulted from (d) norepinephrine, (h) epinephrine or (l) isoprenaline binding to a PBA were demonstrated. Specifically, norepinephrine shows no fluctuation in the low pass portion of the event, but epinephrine and isoprenaline demonstrate minor telegraphic switching. Isoprenaline can be distinguished from epinephrine by recognizing its unique noise characteristics in the high pass portion of the event.

FIG. 51 shows the machine learning workflow. Machine learning was carried out with DarwinML®, a commercial AutoML platform developed based on an evolutionary algorithm for model automation design. To perform the learning process (I), PNRSS measurements were respectively performed with norepinephrine, epinephrine or isoprenaline as the sole analyte (FIGS. 43-48). Raw time traces in abf files were extracted by the neo module (v0.8.0, https://pypi.org/project/neo-python/) in Python. Events in the traces were extracted by a custom event segmentation program, written by Python. The extracted events were then frequency split into the high pass and the low pass portion by a Butterworth filter, integrated in the SciPy module of Python. The cut off frequency was set to 100 Hz and the filter order was set to 2. Standard deviation of the high pass and the low pass portion were respectively calculated and applied to form a feature matrix. 1455 events in the feature matrix was fed into the DarwinML¹⁰² platform for model building. Briefly, 80%, 10% and 10% of the events were respectively used as the training, validation and test data sets. The training and the validation sets were used to build and validate the model. A 10-fold cross validation method was applied. More than 10 popular models, such as SVC (SVM for classification), logistic regression, Random Forest, XGboost, LightGBM, RidgeClassifier, MLPClassifier, BaggingClassifier and some others were applied. When evaluated with the test set (the remaining 10% of all 1455 events), the SVC model reported the highest accuracy score of 99.6%. The trained SVC model was further validated with all 1455 events and an overall accuracy score of 98.3% was reported. The confusion matrix result is demonstrated in FIG. 5f. To perform the predicting process (II), PNRSS was carried out with a sample mixture. The raw current trace was frequency split into the high and the low frequency portions. SVC was applied to label the events (FIG. 5f). To generate the decision boundary, a mesh grid was generated within the area of 0-3 pA in the Lp S.D. and 1-4.5 pA in the Hp S.D. with a 0.01 pA interval. Event type regions can be identified by these mesh grid parameters when inferenced from the SVC model. The boundary separating these regions were taken as the decision boundaries (FIG. 5f).

FIG. 52 shows sequential addition of norepinephrine, epinephrine and isoprenaline. PNRSS measurements were carried out as described in FIG. 5. The buffer applied was 1.5 M KCl, 10 mM HEPES, pH 8.0. A +160 mV potential was continuously applied. Norepinephrine (N), epinephrine (E) and isoprenaline (I) were sequentially added to the trans compartment, reaching a 280 μM, a 280 μM and a 180 μM final concentration respectively. a. A representative trace acquired when only norepinephrine was added. b. The event scatter plot generated from a 15 min continuously recorded trace, acquired as described in a. c. 241 events are included in the scatter plot. A representative trace when epinephrine was further added. d. The corresponding event scatter plot generated from a 15 min trace acquired acquired as described in c. 323 events are included in the scatter plot. e. A representative trace acquired when isoprenaline was further added. f. The corresponding event scatter plot generated from a 15 min trace acquired from the condition described in e. 388 events are included in the scatter plot. Event scatter plots were generated according to the low pass (Lp) and the high pass (Hp) standard deviation (S.D.) values of each event, as described in FIG. 50.

FIG. 53 shows Remdesivir binding to a PBA. a. The schematic diagram. The PNRSS strand 14PBA (Table 1) contains a sole PBA at site 14, capable of binding remdesivir, as illustrated by the cartoon diagram. b. The reactive mechanism. The ribose moiety of remdesivir binds to the PBA, forming a boronate ester¹⁰³. c. Representative traces containing remdesivir binding events. The electrolyte buffer was 1.5 M KCl, 10 mM HEPES, pH 8.0. A +160 mV potential was continuously applied. Remdesivir, dissolved in DMSO with a 10 mM concentration, was added to trans to reach a final concentration of 0-100 μM, marked on the left of each corresponding trace. The rate of event appearance increases when the remdesivir concentration is raised. d. Concentration dependence. The reciprocal of inter-event interval (1/τₒₙ) and the reciprocal of dwell time (1/τ_{off}) is plotted against the final concentration of remdesivir. 1/τₒₙ demonstrates a linear correlation with the concentration of remdesivir. However, 1/τ_{off} of stays constant. e. Scatter plot of ΔI vs. t_{off}. 118 events are included in the scatter plot. The histogram of ΔI, superimposed with its Gaussian fitting result, is plotted to the right of the scatter plot. The remdesivir concentration was 80 μM. The events were extracted from a 15 min continuously recorded trace.

FIG. 54 shows τₒₙ and τ_{off} of remdesivir binding to a PBA. Histograms of the inter-event interval (tₒₙ) and the event dwell time (t_{off}) with different remdesivir concentrations are presented. Remdesivir was added to trans with a final concentration of 20-100 μM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 55 shows Remdesivir metabolite binding to a PBA. a. The schematic diagram. The PNRSS strand 14PBA (Table 1) contains a sole PBA at site 14, capable of binding the remdesivir triphosphate metabolite, as illustrated by the cartoon diagram. b. The reactive mechanism. The ribose moiety of remdesivir triphosphate metabolite binds to the PBA, forming a boronate ester¹⁰³. c. Representative traces containing remdesivir triphosphate metabolite binding events. The electrolyte buffer was 1.5 M KCl, 10 mM HEPES, pH 8.0. Remdesivir triphosphate metabolite, originally dissolved in DMSO at a 10 mM concentation, was added to trans with a final concentration of 0-600 μM, marked on the left of each corresponding trace. A +160 mV potential was continuously applied. The rate of event appearance increases when the remdesivir triphosphate metabolite concentration is raised. d. Concentration dependence. The reciprocal of inter-event interval (1/τₒₙ) and the reciprocal of dwell time (1/τ_{off}) is plotted against the final concentration of remdesivir triphosphate metabolite. 1/τₒₙ demonstrates a linear correlation with the concentration of remdesivir triphosphate metabolite. However, 1/τ_{off} stays constant. e. Scatter plot of ΔI vs. t_{off}. 130 events are included in the scatter plot. The histogram of ΔI, superimposed with its Gaussian fitting result, is plotted to the right of the scatter plot. The remdesivir triphosphate metabolite concentration was 500 μM. The events were extracted from a 15 min continuously recorded trace.

FIG. 56 shows τₒₙ and τ_{off} of remdesivir metabolite binding to a PBA. Histograms of the inter-event interval (tₒₙ) and the event dwell time (t_{off}) with different remdesivir metabolite concentrations are presented. Remdesivir metabolite was added to trans with a final concentration of 200-600 μM. The applied concentration is marked on the left of each corresponding histogram plot. All histograms were respectively fit with a single exponential function y=a*exp(−x/τ), from which the mean inter-event interval (τₒₙ) and the mean event dwell time (τ_{off}) were derived and marked on each corresponding histogram plot. The PNRSS measurements were performed as described in Methods. The PNRSS strand 14PBA (Table 1) was applied. A buffer of 1.5 M KCl, 10 mM HEPES, pH 8.0 was used. A +160 mV potential was continuously applied.

FIG. 57 shows sequential addition of remdesivir metabolite and remdesivir. The PNRSS measurement was carried out as described in FIG. 6. The electrolyte buffer was 1.5 M KCl, 10 mM HEPES, pH 8.0. A +160 mV potential was continuously applied. Remdesivir metabolite (M) and remdesivir (R) were sequentially added to the trans compartment, reaching a 500 μM and a 20 μM final concentration respectively. a. The representative trace acquired when only remdesivir metabolite was added. Events of remdesivir metabolite binding are marked with purple M characters. b. The event scatter plot of low pass (Lp) standard deviation vs. the high pass (Hp) standard deviation from a 15 min continuously recorded trace, as described in a. 100 events are included in the scatter plot. Only remdesivir metabolite binding events (M) were observed in the scatter plot. c. The representative trace acquired when remdesivir was further added. The newly emerged events of remdesivir binding are marked with magenta R characters. d. The event scatter plot of low pass (Lp) standard deviation vs. the high pass (Hp) standard deviation from a 15 min continuously recorded trace, as described in c. 126 events are included in the scatter plot. Two clearly separated event populations are observed from the plot, demonstrating binding of remdesivir metabolite (M) and remdesivir (R) respectively.

FIG. 58 shows demonstration of PNRSS with α-HL. a. The measurement configuration. During PNRSS, an α-hemolysin (α-HL) nanopore serves to dock a streptavidin tethered PNRSS strand. b. The schematic diagram. The PNRSS strand 14PBA (Table 1) contains a sole PBA at site 14, capable of binding an isoprenaline, as illustrated by the cartoon diagram. c. Representative traces containing isoprenaline binding events (purple circle). Even without the addition of isoprenaline, some negative going spiky noises were observed, indicating that the phenylboronic acid has detectable interactions with amino acid residues within the pore lumen. This is however not observed when measured with MspA. Binding of isoprenaline to a PBA results in negative going events. PNRSS measurements were carried out as described in Methods. The electrolyte buffer was 1.5 M KCl, 10 mM HEPES, pH 8.0. A +160 mV potential was continuously applied. Isoprenaline was added to trans with a final concentration of 0-30 μM. The applied concentration is marked on the left of each corresponding trace. The rate of event appearance increases when the isoprenaline concentration is raised. Inset: expanded view of the binding event. d. Concentration dependence. The isoprenaline concentration was modulated between 10-30 μM. 15 min continuous recording was performed for each condition. τₒₙ and τ_{off} values were derived as described in FIG. 10. Mean and standard deviation values were from three independent measurements for each condition. The reciprocal of inter-event interval (1/τₒₙ) and the reciprocal of dwell time (1/τ_{off} are plotted against the final concentration of isoprenaline. 1/τₒₙ demonstrates a linear correlation with the concentration of isoprenaline. However, 1/τ_{off} stays constant. e. Event scatter plot of ΔI vs. t_{off}. The histogram of ΔI, superimposed with its Gaussian fitting result, is plotted to the right of the scatter plot. A single population of events, measuring ˜−4.6 pA in ΔI was identified. The isoprenaline concentration was 30 μM. The events were extracted from a 10 min continuously recorded trace. The number of binding events is 100.

FIG. 59 shows a PNRSS strand with no traction section. a. The schematic diagram of a PNRSS strand 14TAK-NTS (Table 1). 14TAK-NTS has no traction section. b. A representative trace of PNRSS measurement with streptavidin tethered 14TAK-NTS. The measurement was carried out as described in Methods. A buffer of 1.5 M KCl, 10 mM HEPES, pH 8.0 was used. A +160 mV potential was continuously applied. Without the traction section, the streptavidin-tethered 14TAK-NTS can't be efficiently trapped by MspA. Only transient pore blockages were observed. This confirms that the traction section of a PNRSS strand is critical to electrophoretically lead the strand into the pore and to maintain the strand in the pore lumen for a continuous measurement.

FIG. 60 shows catechol binding to a PBA measured at different voltages. a. Representative traces for catechol binding to a PBA when a +80 mV, +100 mV, +120 mV, +140 mV or +160 mV voltage was applied. Catechol in trans was kept at a 200 μM concentration. Binding of catechol to a PBA results in positive going events. The rate of event appearance is generally unchanged when the applied voltage is modulated. The event amplitude (ΔI=I_{b}−Iₚ) increases when the voltage is raised. b. A plot of 1/τₒₙ or 1/τ_{off} vs. the applied voltage. Both 1/τₒₙ and 1/τ_{off} generally stay constant when different voltages were applied. c. Plot of the mean event amplitude (Δ̅I̅) vs. the applied voltage. The mean event amplitude (Δ̅I̅) is larger when a larger voltage is applied. Δ̅I̅ is exponentially related to the applied voltage. The events were extracted from a 15 min continuously recorded trace. Three independent measurements (N=3) were performed for each condition to generate the statistics.

FIG. 61 shows norepinephrine binding to a PBA at different voltages. a. Representative traces for norepinephrine binding to a PBA when a +80 mV, +100 mV, +120 mV, +140 mV or +160 mV voltage was applied. Norepinephrine in trans was kept at a 60 μM concentration. Binding of norepinephrine to a PBA results in negative going events. The rate of event appearance increases when the voltage is raised. The amplitude of event increases when the voltage is raised. b. A plot of 1/τₒₙ or 1/τ_{off} vs. the applied voltages. 1/τₒₙ is linearly correlated to the voltage. However, 1/τ_{off} stays constant. c. Plot of the mean event amplitude (ΔI) vs. the applied voltages. The absolute value of ΔI increases when the voltage is raised and is linearly correlated with the voltage. The events were extracted from a 15 min continuously recorded trace. Three independent measurements (N=3) were performed for each condition to generate the statistics.

FIG. 62 shows norepinephrine binding to a PBA at different salt concentrations. a. Representative traces containing events of norepinephrine binding to a PBA when a 0.5 M, 1.5 M or 2.5 M KCl electrolyte buffer (other components: 10 mM HEPES, pH 8.0) was applied. Norepinephrine in trans was kept at a 60 μM concentration. A +160 mV potential was continuously applied. Binding of norepinephrine to a PBA results in negative going events. The rate of event appearance decreases when the concentration of KCl is raised. However, the amplitude of event increases. b. A plot of I/τₒₙ or 1/τ_{off} vs. the concentration of KCl in electrolyte buffer. 1/τₒₙ is linearly negatively correlated to the [KCl], whereas I/τ_{off} stays constant. c. Plot of the mean event amplitude (ΔI) vs. the [KCl]. The absolute value of ΔI increases when the [KCl] is raised. The events were extracted from a 15 min continuously recorded trace. Three independent measurements (N=3) were performed for each condition to generate the statistics.

FIG. 63 shows norepinephrine binding to a PBA at different temperature. a-e. Representative traces containing events of norepinephrine binding to a PBA when the temperature was set at 5° C., 10° C., 15° C., 20 or 25° C. Norepinephrine in cis was kept at a 400 μM concentration. Binding of norepinephrine to a PBA results in negative going events. The rate of event appearance and the event dwell time increase when the temperature is raised. f. A plot of 1/τₒₙ vs. the temperature. g. A plot of 1/τ_{off} vs. the temperature. Both 1/τₒₙ and 1/τ_{off} are exponentially related to the temperature and could be described by the Arrhenius relation²². h. A plot of K_{b} vs. the temperature. K_{b} decreases with the increase of the temperature. All measurements involved temperature variation were carried out with an Orbit Mini miniaturized bilayer workstation (Nanion Technologies GmbH, Germany) with a 1.25 kHz sampling rate and no further digital filtration. The events were extracted from a 15 min continuously recorded trace. Three independent measurements (N=3) were performed for each condition to generate the statistics.

FIG. 64 shows PNRSS detection of human urine samples. The measurements were carried out as described in Methods. A buffer of 1.5 M KCl, 10 mM HEPES, pH 8.0. was used. A +160 mV potential was continuously applied. A PNRSS strand 14PBA (Table 1) was applied. a. A representative PNRSS trace when no urine sample was added. Positive spiky events were stochastically prior to the addition of any urine sample. b. A representative trace when 20 μL human urine sample was added to trans. c. A representative trace when 50 μL urine sample was added to trans. No extra events were observed from human urine samples, confirming that urine sample is not generating any interfering events for this measurement.

FIG. 65 shows PNRSS detection of vitamin B6 in urine. a. The workflow. The detection is composed of three steps, including urine sample collection (I), premixing different concentrations of vitamin B6 in urine (II) and PNRSS detection (III). The urine sample was collected from a healthy volunteer (Asian, male, age 27). For the purpose of calibration and test of feasibility, vitamin B6 was added to urine samples to reach a final concentration of 10, 15, 20, 25 or 30 μM. 50 μL urine samples containing vitamin B6 were added to trans prior to each PNRSS measurement. b-d. Representative traces acquired with different urine samples. The events of vitamin B6 were marked with purple circles. Inset: an expanded view of the binding events. Generally, the rate of event appearance is increased when a higher concentration of vitamin B6 was added. e. A plot of 1/τₒₙ or 1/τ_{off} vs. the concentration of vitamin B6 in urine. (1/τₒₙ) demonstrates a linear correlation with the concentration of vitamin B6 in urine. (1/τ_{off}) stays constant. f. Scatter plot of ΔI vs. t_{off}. 189 events are included in the scatter plot. The histogram of ΔI, superimposed with its Gaussian fitting result, is plotted to the right of the scatter plot. A single population of events, measuring ˜7.4 pA in ΔI was identified. The concentration vitamin B6 in urine was 40 μM. The events were extracted from a 10 min continuously recorded trace.

FIG. 66 shows PNRSS measurement with polymer PNRSS strand. a. The schematic diagram. The polymer PNRSS strand 14PBA-Spacer9 consists of oligonucleotides and polymer. The polymer unit was formed by the polymerization of three molecules of ethylene glycol, as illustrated by the cartoon diagram. In addition, 14PBA-Spacer9 contains a sole PBA, capable of binding norepinephrine. b. A representative trace when no mobile reactant was added. c. Representative trace containing norepinephrine binding events. A buffer of 1.5 M KCl, 10 mM HEPES, pH 8.0 was used. A +160 mV potential was continuously applied. The experiments were carried out as described in Methods. Norepinephrine was added to trans reaching a final concentration of 0.1 mM. 10 min continuous recording was performed for each condition.

FIG. 67 shows conceptual demonstration of fPNRSS. a. The process diagram of fixed-PNRSS (fPNRSS). The PNRSS strand 14TAK with amino modification was reacted with 4-(azidomethyl) benzeneboronic acid by CuAAC as described in Methods. And then, the product was conjugated to MspA protein containing mutations in cysteine via Sulfo-SMCC linker. b. The measurement configuration. Initially, the pore was unoccupied (i) and an open pore current was reported as I₀(i). The PNRSS strand conjugated to the pore was then captured under 20 mV voltage, causing an immediate drop of the blockage level to Iₚ(ii). c. A representative trace when no mobile reactant was added. d. Representative trace containing norepinephrine binding events. A buffer of 1.5 M KCl, 10 mM HEPES, pH 8.0 was used. A +160 mV potential was continuously applied. The experiments were carried out as described in Methods. Norepinephrine was added to trans reaching a final concentration of 0.2 mM. 10 min continuous recording was performed for each condition.

FIG. 68 shows conceptual demonstration of 1PNRSS. a. The process diagram of locked-PNRSS (1PNRSS). A 1PNRSS strand contains a locked section, capable of forming hairpin structure to avoid 1PNRSS strand escape from the pore. In addition, 1PNRSS strand 14PBA contains a sole PBA, capable of binding norepinephrine. b. The measurement configuration. Initially, the pore was unoccupied (i) and an open pore current was reported as I₀(i). A 1PNRSS strand was then captured by the pore, causing an immediate drop of the blockage level twice (ii and iii) to reach a final blockage level as Iₚ(iii). Among them, the blockage level (ii) was caused by the hairpin structure unzipping, as illustrated by the cartoon diagram. c. Representative trace containing norepinephrine binding events. A buffer of 1.5 M KCl, 10 mM HEPES, pH 8.0 was used. A +160 mV potential was continuously applied. The experiments were carried out as described in Methods. Norepinephrine was added to trans reaching a final concentration of 0.05 mM. 10 min continuous recording was performed for each condition.

FIG. 69 shows saccharide binding to a PBA. a. The schematic diagram. The PNRSS strand 14PBA (Table 1) contains a sole PBA at site 14, capable of binding saccharide, as illustrated by the cartoon diagram. b. A representative trace when no mobile reactant was added. c-e. Representative traces containing D-(−)-Ribose (c), D-Fructose (d), D-(+)-Mannose (e) binding events (purple circle). The structural formulas of saccharide were marked on the left of each corresponding trace. The spiky events in blank (b) were from Tris. Tris is a component used to provide buffering capability and reactive to phenylboronic acid (FIG. S33-S35). However, the events of Tris binding to PBA (b) were apparently different from the events of saccharide binding to PBA (c-e). A buffer of 1.5 M KCl, 10 mM Tris, pH 8.0 was used. A +140 mV potential was continuously applied. The experiments were carried out as described in Methods. D-(−)-Ribose (4 mM, c), D-Fructose (4 mM, d), D-(+)-Mannose (6 mM, e) were added to trans reaching the aforementioned final concentrations. 10 min continuous recording was performed for each condition.

FIG. 70 shows 5′-CMP binding to a PBA. a. The schematic diagram. The PNRSS strand 14PBA (Table 1) contains a sole PBA at site 14, capable of binding cytidine 5′-monophosphate (5′-CMP), as illustrated by the cartoon diagram. b. A representative trace when no mobile reactant was added. c. Representative trace containing 5′-CMP binding events (purple circle). d. Scatter plot of ΔI vs. t_{off}. 65 events are included in the scatter plot. e. The histogram of ΔI superimposed with its Gaussian fitting result. A buffer of 1.5 M KCl, 10 mM HEPES, pH 8.0 was used. A +160 mV potential was continuously applied. The experiments were carried out as described in Methods. 5′-CMP was added to trans reaching a final concentration of 0.8 mM. 10 min continuous recording was performed for each condition.

FIG. 71 shows PNRSS discrimination of enantiomeric norepinephrine. a. The schematic diagram. The PNRSS strand 14PBA (Table 1) contains a sole PBA at site 14, capable of binding L or D-Norepinephrine, as illustrated by the cartoon diagram. b. A representative trace acquired when only L-Norepinephrine was added. L-Norepinephrine binding to a PBA results in only one type of blockage level of I_{b,L}. c. A representative trace when D-Norepinephrine was further added. However, D-Norepinephrine binding results in a new blockage level I_{b,D}, beyond the I_{b,L}. d. The event scatter plot generated from a 10 min continuously recorded trace, acquired as described in b. 136 events are included in the scatter plot. e. The event scatter plot generated from a 10 min continuously recorded trace, acquired as described in c. 199 events are included in the scatter plot. Here, the ability of PNRSS to distinguish enantiomers was verified by L or D-Norepinephrine. Other enantiomeric catecholamines, such as DL-3,4-dihydroxyphenylalanine (DL-DOPA), DL-Epinephrine and DL-Isoprenaline will be also studied by this system in a follow up work. A buffer of 1.5 M KCl, 10 mM HEPES, pH 8.0 was used. A +160 mV potential was continuously applied. The experiments were carried out as described in Methods. L or D-Norepinephrine was added to trans reaching a final concentration of 0.15 mM. 10 min continuous recording was performed for each condition.

FIG. 72 shows catechol-D6 binding to a PBA. a. The schematic diagram. The PNRSS strand 14PBA (Table 1) contains a sole PBA at site 14, capable of binding catechol-D6, as illustrated by the cartoon diagram. Catechol-D6 is a deuterium compound, and deuterium replaces all the hydrogen atoms on catechol. b. A representative trace when no mobile reactant was added. c. Representative trace containing catechol-D6 binding events. A buffer of 1.5 M KCl, 10 mM HEPES, pH 8.0 was used. A +160 mV potential was continuously applied. The experiments were carried out as described in Methods. Catechol-D6 was added to trans reaching a final concentration of 0.3 mM. 10 min continuous recording was performed for each condition. Catechol-D6 was phased from Cambridge Isotope Laboratories, Inc (U.S.A).

FIG. 73 shows polysaccharide sensing with PNRSS. A phenylboronic acid (PBA) was introduced for the detection of saccharides with vicinal diols. Representative polysaccharides containing fructose, such as 4-O-β-d-galactopyranosyl-d-fructofuranose (lactulose, a), 6-O-α-D-glucopyranosyl-D-fructofuranose (isomaltulose, b) and 4-O-b-D-galactosylsucrose (galactosylsucrose, c), all report clear and distinct binding events. The potential vicinal diols for binding are marked with red characters. Among three polysaccharides, galactosylsucrose shows the lowest affinity with PBA. Because the 1,2-cis-diols in fructosyl, which are known to have a high affinity for PBA, have been broken for the formation of glycosidic bonds. The experiments were carried out as described in Methods. Lactulose (a), isomaltulose (b) and galactosylsucrose (c) were added to the trans chamber with a final concentration of 8 mM for each analyte.

FIG. 74 shows 3,4-Dihydroxymandelic acid binding to a PBA. a. The schematic diagram. The PNRSS strand 14PBA contains a sole PBA at site 14, capable of binding 3,4-dihydroxymandelic acid, as illustrated by the cartoon diagram. b. Representative traces containing 3,4-dihydroxymandelic acid binding events. A buffer of 1.5 M KCl, 10 mM HEPES, pH 8.0 was used. A +160 mV potential was continuously applied. 3,4-Dihydroxymandelic acid was added to trans with a final concentration of 0 and 0.4 mM, marked on the left of the corresponding trace. Binding of 3,4-dihydroxymandelic acid results in positive going events (I_{b}>Iₚ). c. Scatter plot of ΔI vs. t_{off}. d. The histogram of ΔI, superimposed with its Gaussian fitting result. The 3,4-dihydroxymandelic acid concentration was 0.4 mM. The events were extracted from a 15 min continuously recorded trace. ΔI=26.6349 pA.

FIG. 75 shows 4-Hydroxy-3-methoxymandelic acid (VMA) binding to a PBA. a. The schematic diagram. The PNRSS strand 14PBA contains a sole PBA at site 14, capable of binding VMA, as illustrated by the cartoon diagram. b. Representative traces containing VMA binding events. A buffer of 1.5 M KCl, 10 mM HEPES, pH 8.0 was used. A +160 mV potential was continuously applied. VMA was added to trans with a final concentration of 0 and 2 mM, marked on the left of the corresponding trace. Binding of VMA results in positive going events (I_{b}>Iₚ). c. Scatter plot of ΔI vs. t_{off}. d. The histogram of ΔI, superimposed with its Gaussian fitting result. The VMA concentration was 2 mM. The events were extracted from a 15 min continuously recorded trace. ΔI=26.4662 pA.

FIG. 76 shows 3,4-Dihydroxyphenylacetic acid binding to a PBA. a. The schematic diagram. The PNRSS strand 14PBA contains a sole PBA at site 14, capable of binding 3,4-dihydroxyphenylacetic acid, as illustrated by the cartoon diagram. b. Representative traces containing 3,4-dihydroxyphenylacetic acid binding events. A buffer of 1.5 M KCl, 10 mM HEPES, pH 8.0 was used. A +160 mV potential was continuously applied. 3,4-Dihydroxyphenylacetic acid was added to trans with a final concentration of 0 and 0.5 mM, marked on the left of the corresponding trace. Binding of 3,4-dihydroxyphenylacetic acid results in positive going events (I_{b}>Iₚ). c. Scatter plot of ΔI vs. t_{off}. d. The histogram of ΔI, superimposed with its Gaussian fitting result. The 3,4-dihydroxyphenylacetic acid concentration was 0.5 mM. The events were extracted from a 15 min continuously recorded trace. ΔI=25.1196 pA.

### DETAILED DESCRIPTION OF THE INVENTION

The embodiments described herein can be understood more readily by reference to the following detailed description, examples, and claims, and their previous and following description. It is to be understood that the embodiments described herein are not limited to the specific uses, methods, and/or products. It is also to be understood that the terminology used herein is for the purpose of describing particular aspects only and is not intended to be limiting.

Further, the following description is provided as an enabling teaching of the various embodiments in their best, currently known aspect. Those skilled in the relevant art will recognize that many changes can be made to the aspects described, while still obtaining the beneficial results of this disclosure. It will also be apparent that some of the desired benefits of the present invention can be obtained by selecting some of the features of the various embodiments without utilizing other features. Accordingly, those who work in the art will recognize that many modifications and adaptations to the various embodiments described herein are possible and can even be desirable in certain circumstances and are a part of the present disclosure. Thus, the following description is provided as illustrative of the principles of the embodiments described herein and not in limitation thereof.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are to disclose and describe the methods and/or materials in connection with which the publications are cited.

Throughout this application, the term “about” is used to indicate that a value includes the standard deviation of error for the system or method being employed to determine the value, for example, the term “about” may refer to a range equal to the particular value plus or minus twenty percent (+/−20%). In any embodiment discussed in the context of a numerical value used in conjunction with the term “about,” it is specifically contemplated that the term “about” can be omitted.

It must be noted that as used herein and in the appended claims, the singular forms “a,” “an,” and “the” include plural referents unless the context clearly dictates otherwise. Thus, for example, reference to “an analyte” includes one analyte and a plurality of different analytes and reference to “the molecule” includes reference to one or more molecules. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “comprise”, “include”, “contain”, “have” and variations of these terms, such as comprising, comprises and comprised, are not intended to exclude further additions, components, integers or steps. These terms also encompass the meaning of “consist of” or “consisting of”.

The term “and/or” refers to any one, any few or all of the elements connected by the term.

It should be understood that the method of the present invention may be performed in vivo, in vitro, or ex vivo. The method of the present invention may be not for the purpose of disease treatment, and/or not for the purpose of disease diagnosis.

The term “modified” or “modifying”, as used herein, is meant a changed state or structure of a molecule of the invention. Molecules may be modified in many ways, including chemically, structurally, and functionally, for example, by introduction of a molecule or a group by covalent attachment.

The term “alkyne” and “alkynyl” can be used interchangeably, and refers to —C≡C—.

The term “azide” and “azido” can be used interchangeably, and refers to —N₃.

Chemical reactions of single molecules, caused by rapid formation or breaking of chemical bonds, are difficult to observe even with state of the art instruments. A biological nanopore can be engineered into a single molecule reactor, capable of detecting the binding of a monatomic ion or the transient appearance of chemical intermediates. Pore engineering of this type is however technically challenging, which has significantly restricted further development of this technique. We propose a versatile strategy, “programmable nano-reactors for stochastic sensing” (PNRSS), by which a variety of single molecule reactions of hydrogen peroxide, metal ions, ethylene glycol, glycerol, lactic acid, vitamins, catecholamines or nucleoside analogues can be observed directly. PNRSS presents a refined sensing resolution which can be further enhanced by an artificial intelligence algorithm. Remdesivir, a nucleoside analogue and an investigational anti-viral drug used to treat COVID-19, can be distinguished from its active triphosphate form by PNRSS, suggesting applications in pharmacokinetics or drug screening.

Chemical reactions of single molecules, caused by rapid formation or breaking of chemical bonds, are difficult to observe even with state-of-the-art instruments. A biological nanopore can be engineered into a single molecule reactor, capable of detecting the binding of a monatomic ion or the transient appearance of chemical intermediates. Pore engineering of this type is however technically challenging, which has significantly restricted further development of this technique. We propose a versatile strategy, “programmable nano-reactors for stochastic sensing” (PNRSS), by which a variety of single molecule reactions of hydrogen peroxide, metal ions, ethylene glycol, glycerol, lactic acid, vitamins, catecholamines or nucleoside analogues can be observed directly. PNRSS presents a refined sensing resolution which can be further enhanced by an artificial intelligence algorithm. Remdesivir, a nucleoside analogue and an investigational anti-viral drug used to treat COVID-19, can be distinguished from its active triphosphate form by PNRSS, suggesting applications in pharmacokinetics or drug screening.

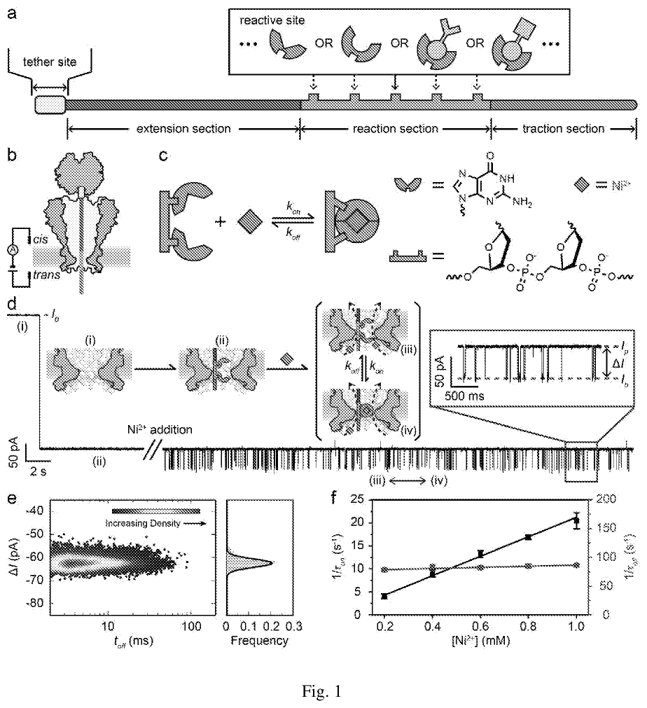

We here present a versatile strategy, “programmable nano-reactors for stochastic sensing” (PNRSS) to democratize nanopore based single molecule chemistry investigations. PNRSS is carried out cooperatively with a strand of synthetic polymer and a nanopore, defined respectively as the PNRSS strand and the PNRSS pore. A PNRSS strand is itself composed of functional modules defined as the tether site, the extension section, the reaction section and the traction section (FIG. 1a). The tether site serves to conjugate one end of the strand to a tether such as a streptavidin. A streptavidin-tethered PNRSS strand is electrophoretically docked, remaining fully stretched in the PNRSS pore (FIG. 1b). This configuration has been previously applied to discriminate between different nucleic acid sequences^{15,33,34} but has never been applied to perform nanopore single molecule chemistry measurements. This configuration has much lowered the technical hurdle of pore engineering. Design of PNRSS strand is fully flexible and the corresponding synthesis can be performed by low-cost commercial services. The length of the extension section of a PNRSS strand is optimized with a 3.5 Å precision so that the reaction section is located at the pore restriction for optimum performance. One or multiple reactive sites within the reaction section form a fixed reactant, which directly participates in the reaction under investigation. The traction section serves to maintain the electrophoretic force on the strand. The mobile reactant, which binds to the fixed reactant, is placed in the measurement environment and acts when bound to the fixed reactant. A PNRSS strand can be composed of any synthetic polymer such as nucleic acid, peptide, polysaccharide or combinations thereof, but to study a wider variety of single molecule reactions, the composition of the PNRSS strand should be arbitrarily programmable. DNA, the most investigated synthetic polymer, can be easily and economically synthesized, chemically modified, enzymatically treated, purified, characterized and stored³⁵. The method is not restricted to DNA but it is an ideal component of a PNRSS strand.

The PNRSS pore should possess a sharp and narrow restriction for a high spatial resolution, a rigid and reproducible structure for a high measurement consistency and a chemically inert pore lumen to minimize undesired reactions. Recent reports of engineered Mycobacterium smegmatis porin A (MspA) in applications of single molecule chemistry have demonstrated its structural superiority by which a significantly enlarged event amplitude (˜55 pA) was reported²⁷. Though not restricted to, an MspA (Methods) is applied to demonstrate all PNRSS measurements in this paper.

In summary, chemical reactions of single molecules, caused by rapid formation or breaking of chemical bonds, are difficult to observe even with state of the art instruments. A biological nanopore can be engineered into a single molecule reactor, capable of detecting the binding of a monatomic ion or the transient appearance of chemical intermediates. Pore engineering of this type is however technically challenging, which has significantly restricted further development of this technique. We propose a versatile strategy, “programmable nano-reactors for stochastic sensing” (PNRSS), by which a variety of single molecule reactions of hydrogen peroxide, metal ions, ethylene glycol, glycerol, lactic acid, vitamins, catecholamines or nucleoside analogues can be observed directly. PNRSS presents a refined sensing resolution which can be further enhanced by an artificial intelligence algorithm. Remdesivir, a nucleoside analogue and an investigational anti-viral drug used to treat COVID-19, can be distinguished from its active triphosphate form by PNRSS, suggesting applications in pharmacokinetics or drug screening.

Nanopore

The term “nanopore”, as used herein, generally refers to a pore, channel or passage which has a very small diameter on the order of nanometres and extends through a membrane. A nanopore may have a characteristic width or diameter on the order of 0.1 nanometres (nm) to about 1000 nm.

The nanopore of the present invention may be in any form and may be a biological nanopore or a synthetic nanopore, the nanopore of the present invention may be, as known by the person skilled in the art. e.g., a solid-state nanopore, a protein nanopore, a hybrid solid state-protein nanopore, or a DNA origami nanopore.

Examples of the protein nanopore comprise alpha-hemolysin (α-L) Mycobacterium smegmatis porin A (MspA), Aerolysin, curli production assembly/transport component (CsgG), outer membrane porin F (OmpF), Cytolysin A (ClyA), ferric hydroxamate uptake component A (FhuA), Fragaceatoxin C (FraC), Pleurotolysin A (PlyA)/Pleurotolysin B(PlyB), Curli production assembly/transport component CsgG (CsgG) and Phi29 connector protein. The protein nanopore may be a naturally occurring wild-type protein nanopore or a homolog or variant of the wild-type protein nanopore.

Sequences of wild type protein nanopore can be found in GenBank on https://www.ncbi.nlm.nih.gov/. For example, the wild-type MspA may have the following amino acid sequence:

GLDNELSLVDGQDRTLTVQQWDTFLNGVFPLDRNRLTREWFHSGRAKYIVAG PGADEFEGTLELGYQIGFPWSLGVGINFSYTTPNILIDDGDITAPPFGLNSVITPNLFPG VSISADLGNGPGIQEVATFSVDVSGAEGGVAVSNAHGTVTGAAGGVLLRPFARLIAST GDSVTTYGEPWNMN (SEQ ID NO:1).

As known by the person skilled in the art, a protein nanopore generally comprises a constriction zone, which is the narrowest portion of the nanopore channel, A protein nanopore may also comprise a vestibule at one end of the nanopore channel, which is also a part of the nanopore channel but has a larger diameter than the constriction zone.

Some protein nanopore may comprise two or more monomers, which associate with each other and form a tunnel, wherein each monomer may be the same of different. Any one of the monomers that formed the protein nanopore may be selected from a wild-type protein, or a homology or a variant thereof. In some embodiments, all monomers in the protein nanopore are the same.

The term “homolog”, as defined herein, is a gene or its protein product that has a similar structure and function with another gene or its protein product. A homolog may have a sequence identity of at least 50%, 55%, 60%, 65%, 70%, 75%, 80%. 85%. 90%, 95%, 96%, 97%, 98%, or 99% compared to its counterpart. The term “homolog” is sometimes used to apply to the relationship between genes or their protein products separated by the event of speciation (see “ortholog”) or to the relationship between genes or their protein products separated by the event of genetic duplication (see “paralog”). The term “ortholog” refers to genes or their protein products in different species that evolved from a common evolutionary origin. The term “paralog” refers to genes related by duplication within a genome.

A variant may have one or more mutations (such as one or more additions, substitutions and/or deletions of amino acids) compared to their wild-type ones, and retains tunnel-forming capability.

The person skilled in the art readily understand how to determine the identity of two polypeptides. For example, the identity can be calculated after aligning two sequences so that the identity is at its highest level. For example, to determine the “percent identity” of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences may be a function of the number of identical positions shared by the sequences (i.e., percent identity=number of identical positions/total number of positions (e.g. overlapping positions)×100). In one embodiment, the two sequences are the same length. Sequence identity can be determined in a number of different manners and through a number of algorithms. To determine sequence identity, sequences can be aligned using various methods and computer programs (e.g. BLAST, T-COFFEE, MUSCLE, MAFFT, etc.), available over the world wide web at sites including ncbi.nlm.nih.gov/BLAST, ebi.ac.uk/Tools/msa/coffee/, ebi.ac.uk/Tools/msa/muscle/, mafft.cbrc.jp/alignment/software/. See, e.g., Altschul et al. (1990), J. Mol. Bioi. 215:403-10.

In some embodiments, the mutations (such as one or more additions, substitutions and/or deletions of amino acids) may be at any site, such as in the surface of the lumen, in the rim or the outside of the periplasmic loops of the protein nanopore. In some embodiments, for a protein nanopore that has a lumen of conical shape, the mutations may in the constriction zone and/or the vestibule of the protein nanopore.

In some embodiments, the variant of the protein nanopore may comprise at least one additional positively charged amino acid, at least one additional negatively charged amino acid, at least one less positively charged amino acid or at least one less negatively charged amino acid compared to its parental protein in the lumen.

In some embodiments, one or more positively charged amino acid in the lumen of the protein nanopore is replaced with a negatively charged amino acid, and each negatively charged anno acid is the same or different; or one or more negatively charged amino acid in the lumen of the protein nanopore is replaced with a positively charged amino acid, and each positively charged amino acid is the same or different.

As an example of the variants, a variant of MspA may comprise (i) mutations such that amino acid positions 90, 91. and 93 contain amino acids with a neutral charge and (ii) one or more mutations at the following amino acid positions: 88, 105, 108, 118, 126, 134, 138 or 139; preferably, a variant MspA may comprise mutations of D90N/D91N/D93N or D93N/D91N/D90N/D118R/D134R/E139K compared to the wild-type MspA. D90N/D91N/D93N or D93N/D91N/D90N/D118R/D134R/E139K means that the mutant comprises simultaneously all of listed six mutations; more preferably, a variant of MspA may only has the mutations of D90N/D91N/D93N (M1 MspA) or D93N/D91N/D90N/D118R/D134R/E139K (M2 MspA) compared to the wild-type MspA. The number used herein identifies the location of site directed mutagenesis, where the first amino acid immediately after the start codon is defined as 1.

In the present invention, the protein nanopore may be recombinant protein.

Examples of the solid-state nanopore comprise nanopores fabricated with solid state materials such as SiNx, graphene, glass, quartz.

Examples of the hybrid nanopores comprise protein nanopores configured in a solid-state membrane or a solid state nanopore having a protein nanopore embedded therein.

The nanopore may be modified, such as be chemically modified. The nanopore may be chemically modified, e.g., on the surface of the lumen of the nanopore, in any way and at any site. The protein nanopore may be chemically modified by attachment of a molecule to one or more amino acids, such as cysteines or lysines. Suitable methods for carrying out such modifications are well-known in the art. The nanopore may be chemically modified by the attachment of any molecule. For instance, the nanopore may be chemically modified by attachment of reactive handle. The protein nanopore may be chemically modified by attachment of or an adaptor that has an effect on the physical or chemical properties of the nanopore, such as cyclodextrin.

Preferably, the protein nanopore used in the present invention does not gate spontaneously, even at 150 mV-200 mV or more. “To gate” or “gating” refers to the spontaneous change of electrical conductance through the tunnel of the protein that is usually temporary (e.g., lasting for as few as 1-10 milliseconds to up to a second). For some protein nanopore, the probability of gating increases with the application of higher voltages. Typically, the protein becomes less conductive during gating, and conductance may permanently stop (i.e., the tunnel may permanently shut) as a result, such that the process is irreversible. Optionally, gating refers to the conductance through the tunnel of a protein spontaneously changing to less than 75% of its open state current.

The preparation method of a nanopore is well known by the person skilled in the art, for example, a protein nanopore could be prepared by prokaryote expression and easily purified by chromatography, and a solid-state nanopore could be prepared via an etching method by focused ion beam and high-energy electron beam.

Polymer Strand (PNRSS Strand)

The PNRSS strand, as used herein, generally is a polymer strand comprising one or more sensing modules, therefore, the PNRSS strand is also be called a polymer strand in the present invention. In the present invention, the PNRSS strand enters the channel of a nanopore. Preferably, the PNRSS strand stretches in the channel of the nanopore. After the target analyte enters the channel of the nanopore, the interaction between the sensing module and the target analyte can cause a blockage of the nanopore which is measurable, such as measured as an ionic current change. The analyte can be characterized by the measurement of the blockage caused by the interaction. The characterization of different analytes can be achieved by using different sensing modules, thereby render the PNRSS strand programmable.

The polymer strand of the present invention may be driven into the nanopore and stretches in the channel of the nanopore in any manner, e.g., by a voltage across the nanopore.

The polymer strand of the present invention comprises at least a tether site and a reaction section. The polymer strand may be charged, for example, positively or negatively.

The tether site is used to tether (or constrain) the polymer strand so that the polymer strand cannot translocate through the nanopore. The tether site may be located at one end or the polymer strand. The polymer strand is tethered so that the reaction section is located in a zone suitable for measurement of a blockage. The polymer strand may be tethered in any suitable way to any suitable substrate via the tether site.

For example, but not a limitation, the substrate may be a stopper molecule. The stopper molecule may have a size that prevent the stopper molecule from passing through the nanopore, preferably entering enter the nanopore. In other word, the size of the stopper molecule or the size of at least a portion of the stopper molecule is larger than the opening of the nanopore). The stopper molecule may have a three-dimensional structure which determine the size of the stopper molecule. The stopper molecule may be any molecule that meets the above size requirements, such as a protein molecule.

Another example of the substrate is the nanopore itself, such as the protein nanopore. The polymer strand may be tethered to any suitable position of the protein nanopore, such as any amino acid at the outside of the channel of the protein nanopore, e.g., the rim or the outside of the periplasmic loops of the protein nanopore.

The polymer strand may be tethered to the substrate in any suitable way, which is known to the person skilled in the person.

As an example, the polymer strand may be tethered to the stopper molecule via a high binding affinity between a protein and a compound. The stopper molecule may be a protein that can specifically bind to a compound (such as a small molecular compound), and the tether site comprises the compound. The polymer strand is tethered to the stopper molecule via the high binding affinity between the protein and the compound. As an example, the stopper molecule may be an antibody of a hapten, and the tether site may comprise the hapten. As an example, the stopper molecule may be a streptavidin, and the tether site may comprise a biotin. As another example, the stopper molecule may be an anti-digoxin antibody, and the tether site may comprise a digoxin.

As another example, the stopper molecule may be any protein comprising a natural amino acid which can react with a small molecule, or the nanopore protein comprises a natural amino acid which can react with a small molecule, and the tether site may comprise the small molecule. Said natural amino acid may located on the surface of the stopper molecule or the nanopore protein. Said natural amino acid may located on the surface of the rim or the outside of the periplasmic loops of the nanopore protein, or on the surface near the opening of the channel of the nanopore protein. The polymer strand may be tethered to the stopper molecule or the nanopore protein via the reaction between the natural amino acid and the small molecule which can react with the natural amino acid, including, but being not limited to, michael addition reaction between the thiol group of cysteine and maleimide or derivative thereof (Nair, D. P et al., 2013, The Thiol-Michael Addition Click Reaction: A Powerful and Widely Used Tool in Materials Chemistry. Chemistry of Materials, 26(1), 724-744), covalently binding of iodoacetamide or derivative thereof to the thiol group of cysteine (Tyagarajan, K. et al., 2003, Thiol-reactive dyes for fluorescence labeling of proteomic samples. ELECTROPHORESIS, 24(14), 2348-2358), thiol-ene reaction between the thiol group of cysteine and a vinyl of a compound (Dondoni, A., 2008, The Emergence of Thiol-Ene Coupling as a Click Process for Materials and Bioorganic Chemistry. Angewandte Chemie International Edition, 47(47), 8995-8997); thiol-thiol reaction between the thiol group of cysteine and a thiol of a compound (Gilbert, H. F, 1995, [2] Thiol/disulfide exchange equilibria and disulfidebond stability. Biothiols Part A Monothiols and Dithiols, Protein Thiols, and Thiyl Radicals, 8-28); reaction between lysine and trimethylammonium nitrofluorobenzene (Sutton, D. A. et al., 1972, Evaluation of 1-fluoro-2-nitro-4-trimethylammoniobenzene iodide, a protein-solubilizing reagent. Biochemical Journal, 130(2), 589-595) or aryl halides (Lautrette, G. et al., 2016, Nitrogen Arylation for Macrocyclization of Unprotected Peptides. Journal of the American Chemical Society, 138(27), 8340-8343); reaction between methionine and oxaziridine or derivative thereof (Lin, Shixian, et al., 2017, Redox-based reagents for chemoselective methionine bioconjugation, Science, 355(6325), 597-602). The natural amino acid reacting with a small molecule compound comprise, but is not limited to, cysteine, lysine, and/or methionine. The small molecule compound reacting with a natural amino acid include, but is not limited to, maleimide or derivative thereof, iodoacetamide or derivative thereof, a small molecular comprising vinyl or thiol, trimethylammonium nitrofluorobenzene, aryl halides, and/or oxaziridine or derivative thereof. Preferably, in the stopper molecule or the nanopore protein, the group of the natural amino acid which can react with the small molecular compound is free. Preferably, in the stopper molecule or the nanopore protein, the natural amino acid and/or the group of the natural amino acid which can react with the small molecular compound is exposed on the surface of the stopper molecule or the nanopore protein.

As an example, D56 of MspA may be mutated to a cysteine, the tether site may comprise a maleimide or derivative thereof, an iodoacetamide or derivative thereof, a small molecular comprising vinyl or thiol, and the polymer strand is tethered to the cysteine at position 56 of MspA.

As another example, the stopper molecule may be any protein with an introduced first reactive handle, or a first reactive handle is introduced into the nanopore protein. For example, a non-natural amino acid comprising a first reactive handle can be incorporated into the stopper molecule or the nanopore protein, e.g., during the artificial synthesis of the protein or by chemical modification of the protein. Therefore, the stopper molecule or the nanopore protein comprises an exposed first reactive handle, the tether site comprises a second reactive handle which can react with the first reactive handle, and the polymer strand is tethered to the stopper molecule or the nanopore protein via the reaction between the first and second reactive handles. Said natural amino acid may located on the surface of the stopper molecule or the nanopore protein. Said first reactive handle may located on the surface of the stopper molecule or on the rim or the outside of the periplasmic loops of the nanopore protein, or on the surface near the opening of the channel of the nanopore protein.

The term “reactive handle”, as used herein, is meant a chemical molecule, a chemical moiety or a chemical group that is exposed and can react with another reactive handle. Reactive handle pair is composed of a first reactive handle and a second reactive handle, wherein the first reactive handle can react with the second reactive handle. Reactive handle pair are known to the person skilled in the art. Reactive handle pair that can be used in the present invention include, but are not limited to, click reaction handles. Examples of reactive handle pair include, but are not limited to, azide and alkyne, which can react with each other by a copper(I)-catalyzed alkyne-azide cycloaddition (CuAAC); azide and difluorinated cyclooctyne, which can react with each other by copper free alkyne-azide cycloaddition; azide and phosphine which can react with each other by staudinger ligation; thiol and alkene, which react with each other by a radical addition; thiol and maleimide, which react with each other by a Michael addition; amine and para-fluoro, which react with each other by a nucleophilic substitution (Becer, Hoogenboom, and Schubert, Click Chemistry beyond Metal-Catalyzed Cycloaddition, Angewandte Chemie International Edition, 2009, 48: 490-4908; Rostovtsev, V. V. et al., 2002, A stepwise Huisgen cycloaddition process: Copper(I)-catalyzed regioselective “ligation” of azides and terminal alkynes. Angew. Chem., Int. Ed. 41, 2596-2599; Tome, C. W. et al., 2002, Peptidotriazoles on solid phase: [1,2,3]-Triazoles by regiospecific copper(I)-catalyzed 1,3-dipolar cycloadditions of terminal alkynes to azides. J. Org. Chem. 67, 3057-3064; Agard, N. J. et al., 2004, A strain promoted [3+2] azide-alkyne cycloaddition for covalent modification of biomolecules in living systems. J. Am. Chem. Soc. 126, 15046-15047; Kohn, M., and Breinbauer, R., 2004, The Staudinger ligation: A gift to chemical biology. Angew. Chem., Int. Ed. 43, 3106-3116). Any one of a reactive handle pair can be used as the first reactive handle or the second reactive handle.

Through the design of the substrate and the tether site, the polymer strand of the present invention can be tethered in a variety of and flexible ways, which are not limited to any of the above examples.

The terms “polypeptide” and “protein”, are used interchangeably and refer to a polymeric form of amino acids of any length, which can include naturally and non-naturally occurring amino acids, chemically or biochemically modified or derivatized amino acids, and polypeptides having modified peptide backbones.

The reaction section may comprise one or more monomers (such as two or more, three or more, four or more, or five or more) and comprise one or more sensing modules (which is also be called fixed reactant in the present invention). Each sensing module may be regarded as a sensing site or reactive site that interacts with single molecule of the target analyte (which is also be called mobile reactant in the present invention). When the reaction section comprises two or more monomers, the two or more monomers polymerize to form a polymer chain. The reaction section may be charged, for example, positively or negatively.

The term “sensing module”, as used herein, refers to a chemical portion that can interact with single molecule of a target analyte. A sensing module may be comprised of one or more (such as two or more) sensing moieties.

The term “moiety”, as used herein, refers to a chemical molecule or any part of a chemical molecule, such as, a functional group. The term “sensing moiety”, as used herein, refers to a chemical molecule or a part of a chemical molecule which interacts with one or two or more binding sites of single molecule of a target analyte. The sensing moiety may be comprised in the side chain of the monomer unit of the reaction section. The term “side chain”, as used herein, refers to a chemical group that is attached to a core part of the molecule called “main chain” or backbone.

The term “interact” or “interaction”, as used herein, may refer to reaction or binding between the sensing module or the sensing moiety and the target analyte, which may be reversible or irreversible. The interaction between the sensing module and the target analyte may cause a change in the ionic current across the nanopore, which is measurable.

A sensing module may consist of only one sensing moiety capable of interacting with single molecule of a target analyte alone, wherein the sensing moiety is called a non-cooperative sensing moiety.

A sensing module may also consist of two or more sensing moieties, wherein the two or more sensing moieties together interact with single molecule of a target analyte and each sensing moiety interacts with one or two or more binding sites of the single molecule. The two or more sensing moieties together interact with single molecule of a target analyte are called cooperative sensing moieties. The cooperative sensing moieties may be comprised in neighboring monomer units. Single molecule of some target analytes may comprise two or more binding sites where the sensing moiety interacts with the target analyte. The two or more binding sites in one molecule may be identical of different from each other, e.g., have identical or different groups or have identical or different bonds. The two or more cooperative sensing moieties in one sensing module may interact with the two or more binding sites in one molecule, respectively. The two or more cooperative sensing moieties in one sensing module may be identical or different from each other, which can be designed according to the binding sites in the target analyte. The analyte molecule can be grasped more easily and strongly by a sensing module consisting of cooperative sensing moieties.

As an example, the reaction section is a nucleic acid and two neighboring purines selected from the group consisting of guanine and adenine may form a sensing module to grasp single molecule of a divalent metal ion, such as Ni²⁺, Co²⁺ or Cu²⁺, Zn²⁺, Cd²⁺, and the two neighboring purines may be the same or different.

The total number of the sensing module within the reaction section may be one to twenty, such as one, two, three, four, five, six, seven, eight, night, ten, eleven, twelve, thirteen, fourteen, fifteen, sixteen, seventeen, eighteen, nineteen or twenty. Two or more sensing modules within the reaction section may be identical or different from each other. In some embodiments, all of the sensing modules within the reaction section are identical. In some embodiments, part of the sensing modules within the reaction section are identical. In some embodiments, all of the sensing modules within the reaction section are different from each other.

The reaction section may comprise two or more sensing modules capable of interacting with different target analytes to facilitate characterization of different target molecules simultaneously or successively.

For example, two or more identical or different sensing modules, or a combination of them may be used, as long as the sensing modules are capable of interacting with different target analytes, then said different target analytes may interact with the two or more sensing modules, respectively, thereby being characterized simultaneously.

For example, two or more different sensing modules that can interact with specific target analyte respectively and have no cross-reactivity with other target analytes to be characterized can be used. In this case, different analytes can not only be characterized simultaneously, but also be characterized in successive rounds respectively. For example, the first target analyte in the first sample can be characterized first, and then the second target analyte in the second sample can be characterized in sequence without changing the PNRSS strand. As an example, the reaction section may comprise a first sensing module which interacts with a first target analyte and a second sensing module which interacts with a second target analyte, and the first sensing module and the second sensing module are different, wherein the first sensing module does not interact with the second target analyte and the second sensing module does not interact with the first target analyte. In the first round of measurement, the first target molecule occupied the first sensing module and is characterized, and in the second round of measurement, although the first sensing module is occupied, the second sensing module is still available, so the second target molecule can interact with the second sensing module and is characterized.

The two or more monomer units containing the sensing moiety may be arranged next to each other, alternatively, the two or more monomer units containing the sensing moiety may be separated by one or more monomer units without a sensing moiety. The reaction section may consist of one or more monomer units containing the sensing moiety, such as one monomer units containing the sensing moiety or two or more monomer units containing the sensing moiety which are arranged next to each other. The reaction section may also comprise one or more monomer units containing the sensing moiety and additional monomer unit without a sensing molecule, such as 1-3 monomer units located on either side of any monomer unit containing the sensing moiety, or 1-3 monomer units located between any two monomer units containing the sensing molecules.

The size (such as the diameter or the width) of the reaction section with the sensing module should be configured that the reaction section can enter and be accommodated in the channel of the nanopore, and the blockage caused by the interaction between the sensing module and the target analyte can be measured.

Although the narrowest region of the nanopore has the highest sensitivity, it should be understood that a measurable blockage can occur in any region of the channel. The quality and resolution of the blockage signal are related to the diameter of the specific region of the channel and the size of the blockage object present in that region. For a designed reaction section, the person skilled in the art should know which region is suitable to accommodate it, e.g., according to the diameter of the channel of the nanopore, the size of the reaction section of the polymer strand and the size of the target analyte. For example, the larger the reaction section of the polymer strand and the target analyte are, the more suitable the channel region with the larger diameter are; the smaller the reaction section of the polymer strand and the target analyte are, the more suitable the channel region with the smaller diameter are. As an example, for some protein analyte with three-dimensional structures, the vestibule of a protein nanopore having a lumen of conical shape may be suitable. As an example, for the target analyte with a small size, the constriction zone of a nanopore having a cylindrical lumen or a protein nanopore having a conical lumen may be suitable. The person skilled in the art can determine which region of the nanopore channel is suitable for obtaining a measurable blockage signal cause by the interaction between the reaction section of the polymer strand and the target analyte. Therefore, the person skilled in the art can determine the position of the reaction section on the polymer strand and/or the length of the reaction section to locate the reaction section at suitable position.

A sensing module may be designed to interact with specific target analyte and a reaction section may be designed to incorporate the sensing module. A suitable monomer unit may be designed to incorporate the sensing moiety constituting the sensing module. The method for preparing the designed reaction section with one or more sensing modules should be known to the person skilled in the art. The designed reaction section with one or more sensing may be prepared in any suitable way, for example by chemical synthesis.

As an example, a monomer comprising a sensing moiety may be used to synthesize the reaction section so that the sensing moiety is incorporated in the reaction section. In the preparation of the reaction section, one or more monomers comprising a sensing moiety are incorporated into the reaction section. Such monomers include, but are not limited to, natural nucleotide (such as guanine nucleotide, adenine nucleotide, thymine nucleotide, cytosine nucleotide, or uracil nucleotide), amino acid (such as alanine, arginine, asparagine, aspartic acid, cysteine, glutamine, glutamic acid, glycine, Histidine, isoleucine, leucine, lysine, methionine, phenylalanine, proline, serine, threonine, tryptophan, tyrosine, valine). As an example, a nucleotide or a nucleotide analog which contains a guanine, an adenine, a thymine, a cytosine, or a uracil may be used as a monomer to synthesize the reaction section, and the guanine, the adenine, the thymine, the cytosine, or the uracil may be used as the sensing moiety or the sensing module. In some embodiments, the synthesized reaction section comprises two neighbouring purines nucleotides selected from the group consisting of guanine nucleotide and adenine nucleotide. The two neighbouring purines constitute the sensing module.

As another example, the monomer used for synthesis may comprise a functional group, and the functional group may be further modified to form a sensing moiety, thereby incorporating the sensing moiety into the reaction section. In the preparation of the reaction section, one or more monomers comprising the functional group are incorporated into the reaction section. Such monomers include, but are not limited to, 5-Ethynyl-dU-CE phosphoramidite. As an example, a monomer containing an alkyne or an azide, such as 5-Ethynyl-dU-CE phosphoramidite, is used for synthesis, and the alkyne or the azide may be further modified by a Huisgen copper (I)-catalyzed azid-alkyne 1,3-dipolar cycloaddition (CuAAC) to form 1,2,3-trizole, which may be used as a sensing moiety or a sensing module.

As another example, the monomer used for synthesis may comprise a first reactive handle, and the first reactive handle may further react with a second reaction handle linked to a sensing moiety, thereby incorporating the sensing moiety into the reaction section. In the preparation of the reaction section, one or more monomers comprising the first reactive handle are incorporated into the reaction section. The term “reactive handle” is defined as above. As an example, a monomer containing an alkyne or an azide, such as 5-Ethynyl-dU-CE phosphoramidite, is used for synthesis, wherein the alkyne or the azide, as the first reactive handle, may further react with the second reactive handle (an azide or an alkyne) linked to a sensing moiety, such as second reactive handle linked to PBA.

The above ways may be used in combination. As an example of the combination of the above ways, the monomer used for synthesis may comprise a functional group, and the functional group may be further modified to form a first reactive handle, and the first reactive handle may further react with a second reaction handle linked to a sensing moiety, thereby incorporating the sensing moiety into the reaction section.

It should be understood that the above-mentioned way of preparing the reaction section having the sensing moiety is merely illustration, and is not intended to limit the scope of the present invention. The sensing moiety may be incorporated in the reaction section in any suitable manner.

The sensing module can be designed to match the target molecule to be characterized. Different target analytes can be characterized by merely changing the sensing module on the PNRSS strand. For example, the target analyte may include, but are not limited to:
ion comprising metal element, wherein the ion comprising metal element may be a cation or an anion, or a polyatomic ion or a monatomic ion, and the ion comprising metal element may be an ion comprising alkaline-earth metal or transition metal, such as AuCl₄⁻, Mg²⁺, Ca²⁺, Ba²⁺, Ni²⁺, Cu²⁺, Co²⁺, Zn²⁺, Cd²⁺, Ag²⁺, Pb²⁺, etc. saccharides such as monosaccharide, oligosaccharide or polysaccharide, wherein the monosaccharide may be selected from ribose, fructose and mannose, e.g., D-(−)-ribose, D-fructose, D-(+)-mannose, the oligosaccharide may be selected from disaccharide and trisaccharide; wherein examples of the disaccharide may comprise 4-O-β-d-galactopyranosyl-d-fructofuranose (lactulose) or 6-O-α-D-glucopyranosyl-D-fructofuranose (isomaltulose); examples of the trisaccharide may comprise 4-O-b-D-galactosylsucrose (galactosylsucrose);
glucoside;
polyphenol, such as anthocyanin or proanthocyanidin;
catecholamine or catecholamine derivative, such as epinephrine, norepinephrine, or isoprenaline;
polyol, such as compound containing two vicinal hydroxyl groups, a 1,2-cis-diol or a 1,3-cis-diol moiety, e.g., 3,4-Dihydroxymandelic acid, 4-Hydroxy-3-methoxymandelic acid (VMA), 3,4-Dihydroxyphenylacetic acid, catechol, ethylene glycol, glycerol, L-lactic acid, or vitamin (such as vitamin C or vitamin B6);
protonated or deprotonated forms of a compound, such as protonated or deprotonated forms of tris;
a compound containing a ribose moiety, such as a nucleotide, nucleoside, analog thereof or monophosphate derivative thereof or polyphosphate derivative thereof, such as ribonucleotide or deoxyribonucleotide, galidesvir, ribavirin, favipiravir-RTP, remdesivir or its triphosphate metabolite, e.g., cytidine 5′-monophosphate (5′-CMP);
hydrogen peroxide;
oligopeptide or cyclopeptide;
buffer reagent, such as tris;
smaller molecular drug such as nucleoside analogue medicines, such as galidesvir, ribavirin, favipiravir-RTP, remdesivir or its triphosphate metabolite;
neurotransmitter such as catecholamine or derivative thereof;
compound with a specific chirality, such as L-Norepinephrine or D-Norepinephrine; analyte containing an isotope, such as catechol-D6 (deuterium replaces all the hydrogen atoms on catechol);
chemical intermediate;
or any combination thereof.

As an example, guanine, adenine, a sensing module consisting of two neighbouring purines selected from the group consisting of guanine and adenine, or 1,2,3-trizole may be used as a sensing module to interact with an ion comprising metal element which is used as a target analyte. The ion comprising metal element may be cation or anion. The ion comprising metal element may be a polyatomic ion or a monatomic ion. The ion comprising metal element may be an ion comprising alkaline-earth metal or transition metal, such as AuCl₄⁻, Mg²⁺, Ca²⁺, Ba²⁺, Ni²⁺, Cu²⁺, Co²⁺, Zn²⁺, Cd²⁺, Ag²⁺, Pb²⁺, etc.

As an example, PBA may be used as a sensing module to interact with the following target analyte:
saccharides such as monosaccharide, oligosaccharide or polysaccharide, wherein the monosaccharide may be selected from ribose, fructose and mannose, e.g., D-(−)-ribose, D-fructose, D-(+)-mannose, the oligosaccharide may be selected from disaccharide and trisaccharide; wherein examples of the disaccharide may comprise 4-O-β-d-galactopyranosyl-d-fructofuranose (lactulose) or 6-O-α-D-glucopyranosyl-D-fructofuranose (isomaltulose); examples of the trisaccharide may comprise 4-O-b-D-galactosylsucrose (galactosylsucrose);
glucoside;
polyphenol, such as anthocyanin or proanthocyanidin;
catecholamine or catecholamine derivative, such as epinephrine, norepinephrine, or isoprenaline;
polyol, such as compound containing two vicinal hydroxyl groups, a 1,2-cis-diol or a 1,3-cis-diol moiety, e.g., 3,4-Dihydroxymandelic acid, 4-Hydroxy-3-methoxymandelic acid (VMA), 3,4-Dihydroxyphenylacetic acid, catechol, ethylene glycol, glycerol, L-lactic acid, or vitamin (such as vitamin C or vitamin B6);
protonated or deprotonated forms of a compound, such as protonated or deprotonated forms of tris;
a compound containing a ribose moiety, such as a nucleotide, nucleoside, analog thereof or monophosphate derivative thereof or polyphosphate derivative thereof, such as ribonucleotide or deoxyribonucleotide, galidesvir, ribavirin, favipiravir-RTP, remdesivir or its triphosphate metabolite, e.g., cytidine 5′-monophosphate (5′-CMP);
hydrogen peroxide;
oligopeptide or cyclopeptide;
buffer reagent, such as tris;
smaller molecular drug such as nucleoside analogue medicines, such as galidesvir, ribavirin, favipiravir-RTP, remdesivir or its triphosphate metabolite;
neurotransmitter such as catecholamine or derivative thereof;
compound with a specific chirality, such as L-Norepinephrine or D-Norepinephrine; analyte containing an isotope, such as catechol-D6 (deuterium replaces all the hydrogen atoms on catechol);
chemical intermediate;
or any combination thereof.

In some embodiments, the polymer strand may further comprise an extension section between the tether site and the reaction section, although the extension section is not necessary.

If the length of the PNRSS strand with only the reaction section and the tether site can be such that the reaction section will be located in a region suitable for measurement of the blockage caused by the interaction between the sensing module and the target analyte after the PNRSS strand is driven into the channel of the nanopore, then the extension section may not be needed. However, if the length of the PNRSS strand with only the reaction section and the tether site is not long enough to enable the reaction section to be located in a region suitable for measurement of the blockage caused by the interaction between the sensing module and the target analyte after the PNRSS strand is driven into the channel of the nanopore, the extension strand can be used to adjust the position of the reaction section in the channel so that the reaction section is located in a suitable position in channel of the nanopore, so that the blockage caused by the interaction between the reaction section and the target analyte can be measured. In preferred embodiments, the extension strand is used to locate the reaction section in the narrowest region of the channel of the nanopore. Therefore, the length of the extension section depends on the actual need. The extension section may comprise one or more monomer units. As an example, the extension section may comprise an oligonucleotide. The length of the oligonucleotide may 1 nt or more, 2 nt or more, 3 nt or more, 4 nt or more, 5 nt or more, 6 nt or more, 7 nt or more, 8 nt or more, 9 nt or more, 10 nt or more, 11 nt or more, 12 nt or more, 13 nt or more, 14 nt or more, or 15 nt or more.

In some embodiments, the polymer strand may further comprise a traction section, although the traction section is not necessary.

The traction section may be on one side of the reaction section oppsite to the tether site, that is, the traction section and the tether site are not on the same side of the reaction section. The traction section may be charged, for example, positively or negatively.

The traction section can be used to hold the polymer strand (especially the reaction section) in the channel of the nanopore. The traction section can hold the reaction section in the region within the channel of the nanopore suitable for measurement of the blockage caused by the interaction between the target analyte and the sensing module. The traction section can prevent the polymer strand from moving in a reverse direction. The traction section can prevent the polymer strand from exiting the entrance of the nanopore. The traction section facilitates stabilizing the reaction section in the channel region suitable for measurement of the blockage caused by the interaction between the reaction section and the target analyte.

By “reverse direction”, it means the direction opposite to the direction of movement of the polymer strand enter the nanopore.

By “exit the entrance of the nanopore”, it means the polymer strand leave the nanopore from the opening where it enters the nanopore.

The traction section is not necessary. Even if there is no traction section to hold the reaction section in the nanopore, the measurement can be achieved, as long as the polymer strand can temporarily enter the nanopore and stretch.

There is no limitation on the form of the traction section, as long as it can perform its function in any way.

As an example, the traction section can be designed as a polymer chain, which can be subjected to electrophoretic force or electroosmotic flow in the electric field applied to the nanopore so as to tend to move to the other side of the nanopore channel (or tend to pass through the nanopore channel), thereby pulling the reaction section. As an example, the traction section may comprise an oligonucleotide. The length of the oligonucleotide may be 10 nt or more, 11 nt or more, 12 nt or more, 13 nt or more, 14 nt or more, 15 nt or more, 16 nt or more, 17 nt or more, 18 nt or more, 19 nt or more, 20 nt or more, 21 nt or more, 22 nt or more, 23 nt or more, 24 nt or more, 25 nt or more, 26 nt or more, 27 nt or more, 28 nt or more, 29 nt or more, 30 nt or more, 31 nt or more, 32 nt or more, 33 nt or more, 34 nt or more, 35 nt or more, 36 nt or more, 37 nt or more, 38 nt or more, 39 nt or more, 40 nt or more, 41 nt or more, 42 nt or more, 43 nt or more, 44 nt or more, 45 nt or more, 50 nt or more, or 55 nt or more.

By “the other side of the channel” or “the other side of the nanopore”, it means the side opposite to the opening where the polymer strand enters the nanopore.

As another example, the traction section may be a coupling site which is used to tether the reaction section to the surface of the nanopore channel. For example, the traction section may be a small molecule that can react with a natural amino acid on the surface of the nanopore channel. The reaction section may be tethered to the surface of the nanopore channel via the reaction between the natural amino acid and the small molecule. Examples of the small molecule that can react with a natural amino acid are described as above.

As another example, a first reactive handle may be introduced to the surface of the nanopore channel and the traction section comprises a second reactive handle. For example, a non-natural amino acid comprising a first reactive handle may be incorporated into the nanopore protein, e.g., during the artificial synthesis of the protein or by chemical modification of the protein, so that the introduced first reactive handle is located on the surface of the nanopore channel. The reaction section may be tethered to the surface of the nanopore channel via the reaction between the first and second reactive handles. Reactive handle is defined as above.

As another example, the traction section can be designed as a polymer chain, and at least a portion of the traction section can be driven to pass through the nanopore in the electric field applied to the nanopore. The portion of traction section passing through the nanopore can form a three-dimension structure outside the nanopore. the three-dimension structure may have a size larger than the exit opening of the nanopore and may prevent said portion of traction section from retracting into the nanopore. The length of the traction section can be designed to ensure that the portion which can form a three-dimensional structure can pass through the nanopore and reach the outside of the nanopore. In some embodiments, the traction section may comprise a nucleic acid. In some embodiments, the three-dimension structure may be a hairpin. In some embodiments, the three-dimension structure may be a hairpin formed from a nucleic acid sequence.

The term “polymer”, as used herein, refers to a molecule comprising two or more monomers linked together by a covalent bond. The term “polymer” includes homopolymer, copolymer, biological polymer such as nucleic acid or peptide.

The terms “polymer strand”, “polymer chain” or “polymer”, as used herein, can be used interchangeably, and can be linear or branched.

The terms “monomer”, “unit”, “monomer unit” and “structural unit” can be used interchangeably in the present invention and refers to a building block of a polymer. The two or more monomers comprised in a polymer strand or a polymer chain may be identical of different, or part of them are identical.

The monomer unit of the polymer strand of the present invention are not limited, and may include nucleotide or analog thereof, abasic, amino acid or analog thereof, monosaccharide, a monomer unit which can be polymerized to form a homopolymer (such as ethylene glycol, which is can be polymerized to form PEG), a monomer unit which can be polymerized to form a copolymer, or any combination thereof. The monomer unit which can be polymerized to form a homopolymer or a copolymer may be a small molecule compound.

The terms “small molecule” and “small molecule compound”, as used herein, can be used interchangeably, and refer to a low molecular weight compound, e.g., <900 daltons, or with a size on the order of 1 nm. A small molecule in the present invention may mean a small molecule other than a nucleotide, an amino acid and/or a monosaccharide.

The term “nucleotide analog” generally is non-naturally occurring and has a modified nucleotide base moiety, a modified pentose moiety, and/or a modified phosphate moiety compared to the naturally occurring nucleotide (A, T, C or G). Examples of nucleotide analog include, but are not limited to, the monomer unit of arabino nucleic acids (ANA), bridged nucleic acid (BNA), cyclohexenyl nucleic acid (CeNA), 2′-fluoroarabino nucleic acids (FANA), glycol nucleic acid (GNA), hexose nucleic acid (HNA), locked nucleic acid (LNA), morpholino, peptide nucleic acid (PNA) or threose nucleic acid (TNA).

The term “amino acid analog” refers to a compound structurally similar to a naturally occurring amino acid wherein either the C-terminal carboxy group, the N-terminal amino group or side-chain functional group has been chemically modified. Amino acid analogs include, but are not limited to, β-amino acids and amino acids where the amino or carboxyl group is substituted with a similar reaction group (for example, the substitution of a primary amine with a secondary or tertiary amine, or the substitution of a carboxyl group with an ester). aspartic acid-(beta-methyl ester) is an amino acid analog of aspartic acid; N-ethylglycine is an amino acid analog of glycine; or alanine carboxamide is an amino acid analog of alanine.

The polymer strand of the present invention may be based on (in other words, essentially consist of) a nucleic acid, nucleic acid analog, a polypeptide, a polysaccharide, a homopolymer (such as polyethylene, PEG), a copolymer or any combination thereof, that is to say, at least 50%, at least 60%, at least 70%, at least 80%, at least 90%, at least 95%, or 100% of the monomer units of the polymer strand can be polymerized to form a nucleic acid, nucleic acid analog, a polypeptide, a polysaccharide, a homopolymer (such as polyethylene, PEG), a copolymer or any combination thereof.

“Nucleic acid analog”, as used herein, refer to are compounds which are analogous (structurally similar) to naturally occurring RNA and DNA. Nucleic acids are chains of nucleotides, which are composed of three parts: a phosphate backbone, a pentose sugar, either ribose or deoxyribose, and one of four nucleobases. An analogue may have any of these altered. Nucleic acid analog may be distinguished from naturally occurring DNA or RNA by changes to the backbone of the molecule. Examples of nucleic acid analog include, but not limit to, arabino nucleic acids (ANA). bridged nucleic acid (BNA), cyclohexenyl nucleic acid (CeNA), 2′-fluoroarabino nucleic acids (FANA), glycol nucleic acid (GNA), hexose nucleic acid (HNA), locked nucleic acid (LNA), morpholino, peptide nucleic acid (PNA), threose nucleic acid (TNA).

Each section of the polymer strand of the present invention (such as the reaction section, the extension section and/or the traction section) may independently be based on (in other words, essentially consist of) a nucleic acid, nucleic acid analog, a polypeptide, a polysaccharide, a homopolymer (such as polyethylene, PEG), a copolymer, or any combination thereof, that is to say, at least 50%, at least 60%, at least 70%, at least 80%, at least 90%, or at least 95% of the monomer units of each of these sections (such as the reaction section, the extension section and/or the traction section) can be polymerized to form a nucleic acid, nucleic acid analog, a polypeptide, a polysaccharide, a homopolymer (such as polyethylene, PEG), a copolymer, or any combination thereof.

The term “nucleic acid analog”, as use herein, refers to a compound which is analogous (structurally similar) to naturally occurring RNA and DNA and consists of nucleotide analogs. Nucleic acid analogues include, but not limit to, arabino nucleic acids (ANA), bridged nucleic acid (BNA), cyclohexenyl nucleic acid (CeNA), 2′-fluoroarabino nucleic acids (FANA), glycol nucleic acid (GNA), hexose nucleic acid (HNA), locked nucleic acid (LNA), morpholino, peptide nucleic acid (PNA) or threose nucleic acid (TNA).

The term “polypeptide”, as used herein, may comprise naturally occurring amino acids and/or amino acid analogs.

In view of the foregoing, each part of the polymer strand, including the tether site, the extension section, the reaction section and/or the traction section, may be designed. Each part of polymer strand may be implemented in any suitable manner, and is not limited to the above-exemplified ones.

System and Method for Characterizing of a Target Analyte

A nanopore may be disposed in a membrane that separates a first conductive liquid medium from a second conductive liquid medium, which may be called a nanopore system. The channel of the nanopore is the only path for the first conductive liquid medium and the second conductive liquid medium to communicate, Generally, a target analyte is added in at least one of the first conductive liquid medium and the second conductive liquid medium. The membrane can be an organic membrane, such as a lipid bilayer, or a synthetic membrane, such as a membrane formed of a polymeric material. The thickness of the membrane through which the nanopore extends can range from 1 nm to around 10 μm.

The preparation of a nanopore system is well known, for example, for a protein nanopore system, when a porin (such as MspA) is placed in any one of a first conductive liquid medium and a second conductive liquid medium separated by a membrane (such as a lipid bilayer), the protein can insert spontaneously into the membrane to form a nanopore.

The polymer strand (the PNRSS strand) may be placed in either side of the nanopore, i.e., the first conductive liquid medium or the second conductive liquid medium. The target analyte may be placed in either side of the nanopore, i.e., the first conductive liquid medium or the second conductive liquid medium. In some embodiments, the polymer strand and the target analyte are placed in the same side or in different sides of the nanopore.

When an electrical potential difference (also called a voltage or an electric field) is applied between the first conductive liquid medium and the second conductive liquid medium (i.e., an electric field or a voltage is applied across the nanopore), an ionic current is generated through the channel of the nanopore, and the polymer strand may be driven into the nanopore from the conductive liquid medium and stretch, e.g., under the action of electrophoretic force and/or electroosmotic flow. The electrical potential difference may be no less than 20 mV, no less than 40 mV, no less than 60 mV, no less than 80 mV, no less than 100 mV, no less than 120 mV, no less than 140 mV, no less than 160 mV, no less than 180 mV or no less than 200 mV; or range from about 20 mV to 200 mV, range from about 40 mV to 180 mV, range from about 600 mV to 180 mV, range from about 80 mV to 180 mV, range from about 100 mV to 180 mV, range from about 120 mV to 180 mV, range from about 140 mV to 180 mV, range from about 160 mV to 180 mV.

In some embodiments, the electrical potential difference between the first conductive liquid medium and the second conductive liquid medium varies or remains constant. Process and apparatus for applying an electric field to a nanopore are known to the person skilled in the art. For example, a pair of electrodes may be used to applying an electric field to a nanopore. As will be understood, the voltage range that can be used can depend on the type of nanopore system and the analyte being used.

The nanopore system in combination with the polymer strand may be used to characterize (or identify) a target analyte. First, the polymer strand is driven into the channel of the nanopore and stays in the channel, and then the target analyte is driven into the nanopore and interacts with the sensing module on the polymer strand. This interaction leads to a blockage which is measured to characterize the target analyte. A system for characterization of a target analyte may further comprise the target analyte. Optionally, in the system, the target analyte may have interacted with the sensing module, or the target analyte may have not interacted with the sensing module.

The target analyte may be driven into the nanopore by an electrophoretic force or a concentration difference (diffusion effect). The target analyte interacts with the sensing module present in the channel of the nanopore and the interaction causes a blockage of the ionic current, which is measurable, for example, by measuring the current after the target analyte enters the nanopore and comparing it with the current when the polymer strand has entered the nanopore and the target analyte has not entered the nanopore. The blockage of the ionic current may be related to the identity of the target analyte, the interaction between die target analyte with an agent (such as the sensing module), die binding kinetics of the target analyte, etc.

In general, a “blockage of the ionic current” may also be called a “blockade current”, which is evidenced by a change in ionic current that is clearly distinguishable from noise fluctuations and is usually associated with the presence of an analyte molecule within the nanopore. The strength of the blockade, or change in current, will depend on a characteristic of the analyte. More particularly, “blockage” may refer to an interval where the ionic current drops to a level which is about 5-100% lower than the unblocked current level, remains there for a period of time, and returns spontaneously to the unblocked level. For example, the blockade current level may be about, at least about, or at most about 5%, 10%, 15%, 20%, 25%, 30%, 35%, 40%, 45%, 50%, 55%, 60%, 65%, 70%, 75%, 80%, 85%, 90%, 95%, or 100% lower than the unblocked current level. A blockage may be called a blockade event or an event.

The measurement can be performed at any suitable temperature, such as −4° C.-100° C., e.g., 4° C.-50° C., 5° C.-25° C. or room temperature.

After the measurement is completed, a reverse voltage can be applied to drive the polymer strand to move in a reverse direction and exit the nanopore. Then, the voltage direction may be changed again, another polymer strand whose sensing module is not occupied may be driven into the channel of the nanopore and the next measurement can be performed. Therefore, the method of the present invention can repeatedly use the same system for multiple measurements.

Measurement of the current through a nanopore are well known in the art and may be performed by way of optical signal or electric current signal. For example, one or more measurement electrodes could be used to measure the current through the nanopore. These can be, for example, a patch-clamp amplifier or a data acquisition device.

A “liquid medium” includes aqueous, organic-aqueous, and organic-only liquid media. Organic media include, e.g., methanol, ethanol, dimethylsuilfoxide, and mixtures thereof. Liquids employable in methods described herein are well-known in the art. Descriptions and examples of such media, including conductive liquid media, are provided in U.S. Pat. No. 7,189,503, for example, which is incorporated herein by reference in its entirety. Salts, detergents, or buffers may be added to such media. Such agents may be employed to alter pH or ionic strength of the liquid medium. In some embodiments, the salt may comprise KCl and/or the concentration of the salt may be 0.5 M-2.5M. In some embodiments, the concentration of KCl is 1.5 M. The buffering agent may be HEPES or Tris, etc. The pH of the first conductive liquid medium and/or the second conductive liquid medium may be 1.0-13.0, preferably 6.0-8.0, preferably 7.0-7.4, which may depend on the desired charge properties of the target analyte. In some embodiments, the first conductive liquid medium and/or the second conductive liquid medium does not contain Tris.

A current pattern and a current trace, as used herein, may be used interchangeably, refer to the ionic current over time. A current pattern may contain one or more types of blockade event, and may contain one or more individual blockade events of the same type. Characteristics about distribution, frequency, amplitude, etc. of the blockade events can be learned from the current pattern.

In the present invention, the nanopore has different states during the measurement. State I represents an unoccupied nanopore, at which the measured current is the open pore current (I₀). State II and III represents a nanopore occupied with a PNRSS strand, in which the sensing moiety of the PNRSS strand either does not interact (II) or interact (iii) with a target molecule. The measured current at state (II) or (III) is respectively defined as first blockade current (Iₚ) or second blockade current (I_{b})

“Event”, as used herein, refers to a blockage of the nanopore by a target analyte (i.e., an interval where the ionic current drops to a level which is about 5-100% lower than the first blockade current level, remains there for a period of time, and returns spontaneously to the first blockade current level), and also refers to a current change caused by the blockage of the target analyte. The first blockade current level, as used herein, refers to a current level measured when the nanopore occupied with a PNRSS strand, in which the sensing module of the PNRSS strand does not interact with a target analyte molecule. The person skilled in the art know how to determine the occurrence of an event.

A variety of characteristic parameters can be obtained from the current pattern. The characteristic parameters include, but not limit to, open pore current (Iₒ), first blockade current amplitude (Iₚ), second blockade current amplitude (I_{b}), event amplitude (ΔI, defined as ΔI=I_{b}−Iₚ), inter-event duration (tₒₙ), event dwell time (t_{off}), mean event amplitude (Δ̅I̅). One or more of these characteristic parameters can be used to characterize an analyte.

The characterization of the target analyte may include, but is not limited to, determining the identity of the target analyte, determining whether the target analyte is a specific substance, determining the presence or absence of the target analyte, determining the interaction of the target analyte and an agent (for example, the agent may be the sensing module, and the system and the method of the present may be used to determine whether there is an interaction between the target analyte and the sensing moiety), or measuring the binding kinetics of the target analyte and an agent (for example, the agent may be the sensing module, and the system and the method of the present may be used to determine the binding kinetics of the target analyte and the sensing moiety). The identity may include, but is not limited to, what the analyte is, the structure of the analyte, the protonation state or the deprotonation state of the analyte, the chirality of the analyte, etc.

As an example, to determine the identity of the target analyte, a tested current pattern may be compared with a reference current pattern and the identity of the target analyte is determine.

As an example, to determine whether the target analyte and an agent interact with each other, the agent may be comprised in the reaction section of the polymer strand as a sensing module, and occurrence of an event represent the interaction between the target analyte and the agent.

A tested current pattern, as used herein, refers to the current pattern obtained by using the tested analyte (i.e., the target analyte).

A reference current pattern refers the current pattern used as a reference to determine at least one characteristic of the target analyte. According to the purpose of detection, different reference current pattern can be used. For example, the reference current pattern can be a current pattern obtained by using a known analyte under the same conditions with the tested current pattern. It can be determined whether the tested analyte is the same with or different from the reference analyte.

In some embodiments, the characterization of the target analyte according to the tested current pattern may be achieved by using machine learning algorithm.

In some embodiments, the tested current pattern may be filtered to obtain a high pass and/or a low pass, and the tested current pattern is provided from the high pass and/or the low pass. In some embodiments, the cut off frequency of the high pass and/or the low pass is about 100 Hz, the cut off frequency of the high pass and/or the low pass is about 100 Hz.

The system and method of the present invention can be used to characterize the target analyte of single molecule. A large number of analytes can be characterized by the system and method of the present invention, as long as the size of the analyte allows it to enter the channel of the nanopore. If the analyte can interact with a moiety, the analyte can be characterized through the system and method of the present invention, using the moiety as the sensing module.

The system and method of the present invention may be used to characterize multiple different target analytes, such as, by using a polymer strand comprising multiple (such as two or more) sensing modules which can interact with multiple (such as two or more) different target analytes. In some embodiments, multiple different target analytes may be driven to enter the channel of the nanopore simultaneously, and interact with the multiple sensing modules, respectively. The resulting multiple interactions may be measured simultaneously and be distinguished from each other according to their respective current patterns. In some embodiments, multiple different target analytes may be driven to enter the channel of the nanopore in different rounds for measurement. For example, the multiple sensing modules are different from each other and each sensing module can specifically interact with one or more specific analytes. Such a design of sensing modules may be used to characterize multiple different target analytes, wherein each target analyte can only interact with one of the multiple sensing modules. As an illustration, a first target analyte may be driven to enter the channel of the nanopore and interact with a first sensing module, and a first interaction between the first target analyte and the first sensing module is measured; then, a second target analyte may be driven to enter the channel of the nanopore and interact with a second sensing module, and a second interaction between the second target analyte and the second sensing module is measured; the above steps are repeated for other target analytes until all target analytes are characterized or all sensing modules are occupied.

In the system and the method of the present invention, multiple nanopores can be used simultaneously, which can increase the detection limit of the analyte. Preferably, the multiple nanopores are the same.

The present invention also relates to the following aspects:

Aspect 1. A system for identifying a target analyte, comprising:
(1) a nanopore;
(2) a polymer strand composed of a tether site, an extension section, a reaction section and a traction section in turn;
wherein the polymer strand is coupled with a tethering molecule at the tether site, and the tethering molecule has a size larger than the nanopore cavity so that it cannot enter the nanopore cavity;
wherein the extension section has a suitable length so that when the polymer strand is loaded into the nanopore, the reaction section is located at the narrowest part of the nanopore;
wherein the reaction part comprises a first active group capable of binding to the target analyte.

Aspect 2. The system according to aspect 1, wherein the first active group is comprised in the polymer backbone.

Aspect 3. The system according to aspect 2, wherein the polymer backbone comprises one or more polymer monomer derivatives and the first active group is comprised in the one or more polymer monomer derivatives.

Aspect 4. The system according to aspect 1, wherein the first active group is linked to the polymer backbone directly or by one or more linkers.

Aspect 5. A system for identifying a target analyte, comprising:
(1) a nanopore;
(2) a polymer strand composed of a tether site, an extension section, a reaction section and a traction section in turn;
wherein the polymer strand is coupled with a tethering molecule at the tether site, and the tethering molecule has a size larger than the nanopore cavity so that it cannot enter the nanopore cavity;
wherein the extension section has a suitable length so that when the polymer strand is loaded into the nanopore, the reaction section is located at the narrowest part of the nanopore;
wherein the reaction part comprises a second active group, and the second active group is capable of binding to a compound comprising a first active group directly of by one or more linkers; wherein the first active group is capable of binding to the target analyte.

Aspect 6. The system according to aspect 5, wherein the second active group is comprised in the polymer backbone.

Aspect 7. The system according to aspect 6, wherein the polymer backbone comprises one or more polymer monomer derivatives and the first active group is comprised in the one or more polymer monomer derivatives.

Aspect 8. The system according to aspect 5, wherein the second active group is linked to the polymer backbone directly or by one or more linkers.

Aspect 9. The system according to any one of preceding aspects, wherein the system further comprises two compartments separated by an interface; wherein each of the two compartments comprises a liquid medium and the nanopore is in the interface.

Aspect 10. The system according to any one of preceding aspects, wherein the polymer backbone is nucleic acid, peptide, polysaccharide or any combination thereof.

Aspect 11. The system according to any one of preceding aspects, wherein the polymer backbone is DNA, RNA, or a hybrid of DNA and RNA.

Aspect 12. The system according to any one of preceding aspects, wherein the reaction section comprises guanine, adenine or any combination thereof.

Aspect 13. The system according to any one of preceding aspects, wherein the reaction section comprises neighbouring purines selected from the group consisting of guanine and adenine, preferably two neighbouring purines selected from the group consisting of guanine and adenine.

Aspect 14. The system according to any one of preceding aspects, wherein the first active group is 1,2,3-triazole.

Aspect 15. The system according to any one of preceding aspects, wherein 1,2,3-triazole is produced by conjugating an azide to an alkyne via a Huisgen copper (I)-catalyzed azide-alkyne 1,3-dipolar cycloaddition (CuAAC) reaction.

Aspect 16. The system according to any one of preceding aspects, wherein the alkyne is comprised in the polymer backbone, or a compound comprising the alkyne is linked to the polymer backbone directly or by one or more linkers.

Aspect 17. The system according to any one of preceding aspects, wherein the polymer backbone comprises 5-Ethynyl-dU-CE phosphoramidite.

Aspect 18. The system according to any one of preceding aspects, wherein the azide is 3-azidopropylamine.

Aspect 19. The system according to any one of preceding aspects, wherein the first active group is phenylboronic acid.

Aspect 20. The system according to any one of preceding aspects, wherein phenylboronic acid is produced by conjugating 4-(azidomethyl)benzeneboronic acid to an alkyne via CuAAC.

Aspect 21. The system according to any one of preceding aspects, wherein the second active group is an alkyne.

Aspect 22. The system according to any one of preceding aspects, wherein the alkyne is comprised in the polymer backbone, or a compound comprising the alkyne is linked to the polymer backbone directly or by one or more linkers.

Aspect 23. The system according to any one of preceding aspects, wherein the polymer backbone comprises 5-Ethynyl-dU-CE phosphoramidite.

Aspect 24. The system according to any one of preceding aspects, wherein the target analyte is selected from the group consisting of a metal ion, saccharide, catecholamine, catecholamine derivative, a compound containing a 1,2-cis-diol or a 1,3-cis-diol moiety, polyol such as catechol, ethylene glycol, glycerol, L-lactic acid, vitamin such as vitamin C or vitamin B6, buffer reagent such as protonated or deprotonated forms of tris, epinephrine, norepinephrine, isoprenaline, anti-viral medicine such as Remdesivir or triphosphate metabolite of Remdesivir, hydrogen peroxide, polysaccharide or cyclopeptide.

Aspect 25. The system according to any one of preceding aspects, wherein the metal ion is transition metal ion, preferably Ni²⁺, Zn²⁺, Cd²⁺, Co²⁺, or Cu²⁺.

Aspect 26. The system according to any one of preceding aspects, wherein the target analyte is polyol and the liquid medium does not contain tris buffer.

Aspect 27. The system according to any one of preceding aspects, wherein the tethering molecule is a streptavidin.

Aspect 28. The system according to any one of preceding aspects, wherein the tether site is modified with a 5′ biotin-TEG.

Aspect 29. The system according to any one of preceding aspects, wherein the second active group is capable of binding to a variety of compounds containing different first active groups.

Aspect 30. The system according to any one of preceding aspects, wherein the target analyte is a single molecule, a monatomic ion or a chemical intermediate.

Aspect 31. The system according to any one of preceding aspects, wherein the target analyte is a monatomic ion or a chemical intermediate.

Aspect 32. The system according to any one of preceding aspects, wherein the nanopore is protein nanopore, a solid nanopore or a DNA nanopore.

Aspect 33. The system according to any one of preceding aspects, wherein the nanopore has a cavity of conical shape or a cylindrical shape.

Aspect 34. The system according to any one of preceding aspects, wherein the protein nanopore is MspA, M2 MspA mutant (D93N/D91N/D90N/D118R/D134R/E139K), α-HL, aerolysine, ClyA, FraC, PlyA/B or Phi 29 connector.

Aspect 35. The system according to any one of preceding aspects, wherein the nanopores is fabricated with solid state materials such as SiNx, graphene, glass, quartz or DNA frameworks.

Aspect 36. Method for identifying a target analyte, the method comprising:
Providing a nanopore;
Providing a polymer strand composed of a tether site, an extension section, a reaction section and a traction section in turn; wherein the polymer strand is coupled with a tethering molecule at the tether site, and the tethering molecule has a size larger than the nanopore cavity so that it cannot enter the nanopore cavity; wherein the extension section has a suitable length so that when the polymer strand is loaded into the nanopore, the reaction section is located at the narrowest part of the nanopore; and wherein the reaction part has an active group capable of binding to the target analyte;
Allowing the polymer strand to enter the nanopore and positioning the reaction section at the narrowest part of the nanopore;
Allowing the target analyte translocate across the nanopore; and
Measuring the change of the ionic current through the nanopore during the translocation, thereby identifying the target analyte.

Aspect 37. The method according to aspect 36, wherein the first active group is comprised in the polymer backbone.

Aspect 38. The method according to aspect 37, wherein the polymer backbone comprises one or more polymer monomer derivatives and the first active group is comprised in the one or more polymer monomer derivatives.

Aspect 39. The method according to aspect 36, wherein the first active group is linked to the polymer backbone directly or by one or more linkers.

Aspect 40. Method for identifying a target analyte, the method comprising: Providing a nanopore; Providing a polymer strand composed of a tether site, an extension section, a reaction section and a traction section in turn; wherein the polymer strand is coupled with a tethering molecule at the tether site, and the tethering molecule has a size larger than the nanopore cavity so that it cannot enter the nanopore cavity; wherein the extension section has a suitable length so that when the polymer strand is loaded into the nanopore, the reaction section is located at the narrowest part of the nanopore; wherein the reaction part comprises a second active group and the second active group is capable of binding to a compound comprising a first active group directly of by one or more linkers; and wherein the first active group is capable of binding to the target analyte;
Combining the second active group with a compound comprising the first active group directly of by one or more linkers;
Allowing the polymer strand to enter the nanopore and positioning the reaction section at the narrowest part of the nanopore;
Allowing the target analyte translocate across the nanopore; and
Measuring the change of the ionic current through the nanopore during the translocation, thereby identifying the target analyte.

Aspect 41. The method according to aspect 40, wherein the second active group is comprised in the polymer backbone.

Aspect 42. The method according to aspect 41, wherein the polymer backbone comprises one or more polymer monomer derivatives and the first active group is comprised in the one or more polymer monomer derivatives.

Aspect 43. The method according to aspect 40, wherein the second active group is linked to the polymer backbone directly or by one or more linkers.

Aspect 44. Method for identifying multiple target analyte, the method comprising:
(1) providing two compartments separated by an interface; wherein each of the two compartments comprises a liquid medium and the interface has a nanopore;
(2) Providing a polymer strand composed of a tether site, an extension section, a reaction section and a traction section in turn; wherein the polymer strand is coupled with a tethering molecule at the tether site, and the tethering molecule has a size larger than the nanopore cavity so that it cannot enter the nanopore cavity; wherein the extension section has a suitable length so that when the polymer strand is loaded into the nanopore, the reaction section is located at the narrowest part of the nanopore; and wherein the reaction part has an active group capable of irreversibly binding to the first target analyte;
(3) applying a first voltage between the two compartments, thereby causing the polymer strand to enter the nanopore and positioning the reaction section at the narrowest part of the nanopore;
(4) Allowing the first target analyte translocate across the nanopore; and
(5) Measuring the change of the ionic current through the nanopore during the translocation, thereby identifying the first target analyte;
(6) applying a second voltage between the two compartments in the opposite direction to the first voltage, thereby causing the polymer strand irreversibly bound to the first target analyte to leave the nanopore;
(7) repeating (3)-(5) to reload another polymer strand into the nanopore and identify another target analyte.

Aspect 45. The method according to any one of preceding aspects, wherein the method further comprises providing two compartments separated by an interface; wherein each of the two compartments comprises a liquid medium and the nanopore is in the interface.

Aspect 46. The method according to any one of preceding aspects, wherein the polymer backbone is nucleic acid, peptide, polysaccharide or any combination thereof.

Aspect 47. The method according to any one of preceding aspects, wherein the polymer backbone is DNA, RNA, or a hybrid of DNA and RNA.

Aspect 48. The method according to any one of preceding aspects, wherein the reaction section comprises guanine, adenine or any combination thereof.

Aspect 49. The method according to any one of preceding aspects, wherein the reaction section comprises neighbouring purines selected from the group consisting of guanine and adenine, preferably two neighbouring purines selected from the group consisting of guanine and adenine.

Aspect 50. The method according to any one of preceding aspects, wherein the first active group is 1,2,3-triazole.

Aspect 51. The method according to any one of preceding aspects, wherein 1,2,3-triazole is produced by conjugating an azide to an alkyne via a Huisgen copper (I)-catalyzed azide-alkyne 1,3-dipolar cycloaddition (CuAAC) reaction.

Aspect 52. The method according to any one of preceding aspects, wherein the alkyne is comprised in the polymer backbone, or a compound comprising the alkyne is linked to the polymer backbone directly or by one or more linkers.

Aspect 53. The method according to any one of preceding aspects, wherein the polymer backbone comprises 5-Ethynyl-dU-CE phosphoramidite.

Aspect 54. The method according to any one of preceding aspects, wherein the azide is 3-azidopropylamine.

Aspect 55. The method according to any one of preceding aspects, wherein the first active group is phenylboronic acid.

Aspect 56. The method according to any one of preceding aspects, wherein phenylboronic acid is produced by conjugating 4-(azidomethyl)benzeneboronic acid to an alkyne via CuAAC.

Aspect 56. The method according to any one of preceding aspects, wherein the second active group is an alkyne.

Aspect 58. The method according to any one of preceding aspects, wherein the alkyne is comprised in the polymer backbone, or a compound comprising the alkyne is linked to the polymer backbone directly or by one or more linkers.

Aspect 59. The method according to any one of preceding aspects, wherein the polymer backbone comprises 5-Ethynyl-dU-CE phosphoramidite.

Aspect 60. The method according to any one of preceding aspects, wherein the target analyte is selected from the group consisting of a metal ion, saccharide, catecholamine, catecholamine derivative, a compound containing a 1,2-cis-diol or a 1,3-cis-diol moiety, polyol such as catechol, ethylene glycol, glycerol, L-lactic acid, vitamin such as vitamin C or vitamin B6, buffer reagent such as protonated or deprotonated forms of tris, epinephrine, norepinephrine, isoprenaline, anti-viral medicine such as Remdesivir or triphosphate metabolite of Remdesivir, hydrogen peroxide, polysaccharides or cyclopeptide.

Aspect 61. The method according to any one of preceding aspects, wherein the metal ion is transition metal ion, preferably Ni²⁺, Zn²⁺, Cd²⁺, Co²⁺, or Cu²⁺.

Aspect 62. The method according to any one of preceding aspects, wherein the target analyte is polyol and the liquid medium does not contain tris buffer.

Aspect 63. The method according to any one of preceding aspects, wherein the tethering molecule is a streptavidin.

Aspect 64. The method according to any one of preceding aspects, wherein the tether site is modified with a 5′ biotin-TEG.

Aspect 65. The method according to any one of preceding aspects, wherein the second active group is capable of binding to a variety of compounds containing different first active groups.

Aspect 66. The method according to any one of preceding aspects, wherein the target analyte is a single molecule, a monatomic ion or a chemical intermediate.

Aspect 67. The method according to any one of preceding aspects, wherein the target analyte is a monatomic ion or a chemical intermediate.

Aspect 68. The method according to any one of preceding aspects, wherein the nanopore is protein nanopore, a solid nanopore or a DNA nanopore.

Aspect 69. The method according to any one of preceding aspects, wherein the nanopore has a cavity of conical shape or a cylindrical shape.

Aspect 70. The method according to any one of preceding aspects, wherein the protein nanopore is MspA, M2 MspA mutant (D93N/D91N/D90N/D118R/D134R/E139K), α-HL, aerolysine, ClyA, FraC, PlyA/B or Phi 29 connector.

Aspect 71. The method according to any one of preceding aspects, wherein the nanopores is fabricated with solid state materials such as SiNx, graphene, glass, quartz or DNA frameworks.

Aspect 72. The method according to any one of preceding aspects, which is used for screening drugs.

Aspect 73. Use of a system according to any one of aspects 1-35 in screening drugs.

### Example 1 PNRSS with Natural DNA Bases

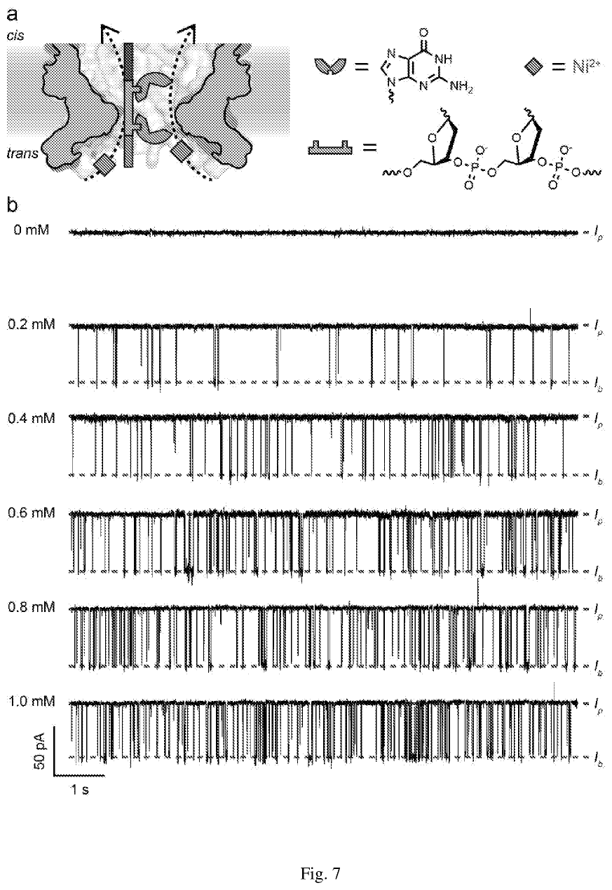

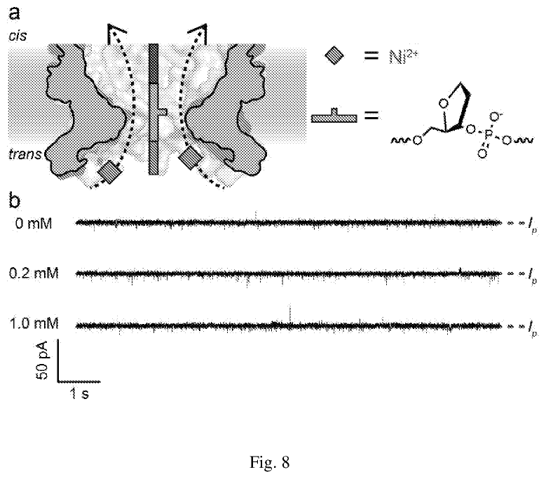

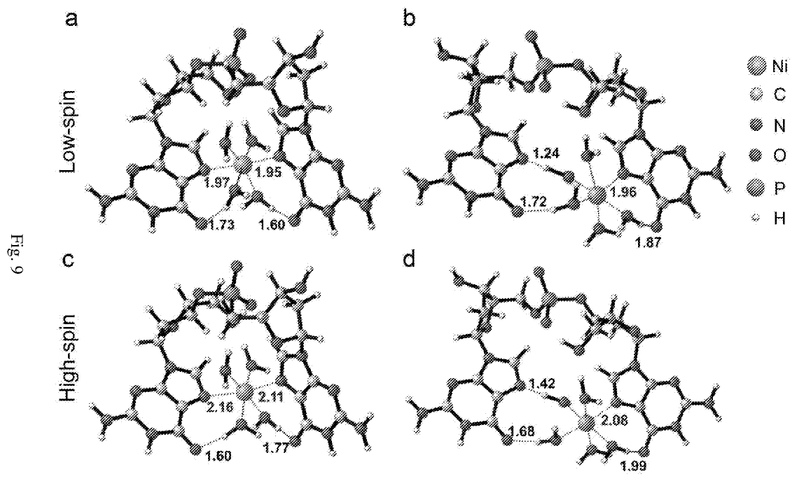

Natural nucleic acid bases, such as guanine, adenine or any combination thereof, can act as a coordination ligand which can bind a metal ion^{36,37}. A first PNRSS strand 13G/14G (Table 1) had two neighbouring guanines that cooperatively bind a Ni²⁺ ion (FIG. 1c). To avoid interferences from other DNA bases, these guanines were surrounded by abasic residues which can not bind metal ions. The PNRSS measurement was carried out as described in Methods. This 13G/14G strand was added to the cis chamber with a 10 nM final concentration. With a single pore inserted and a +180 mV potential continuously applied, an open pore current I₆ was initially reported. Subsequently, a PNRSS strand was captured electrophoretically and reported a static residual current, Iₚ (Table 2). Ni²⁺, serving as the mobile reactant, was then added to the trans chamber with a 1 mM final concentration. This immediately results in further blockage events, reaching a level defined as I_{b}. Successive appearances of events were observed as telegraphic switching between Iₚ and I_{b}. (FIG. 1d, Video 1). The concentration of Ni²⁺ in trans was adjusted between 0-1 mM (FIG. 7), and then the rate of event appearance clearly increased at a higher [Ni²⁺ ]. These events were however not observed from another PNRSS strand 14X (FIG. 8), confirming that they originate from binding to the dual guanine ligand³⁷. This phenomenon was also theoretically simulated, and the N(7) and the 0(6) atoms were seen to play a critical role in the coordination of a Ni²⁺ (FIG. 9, Table 3, Methods).

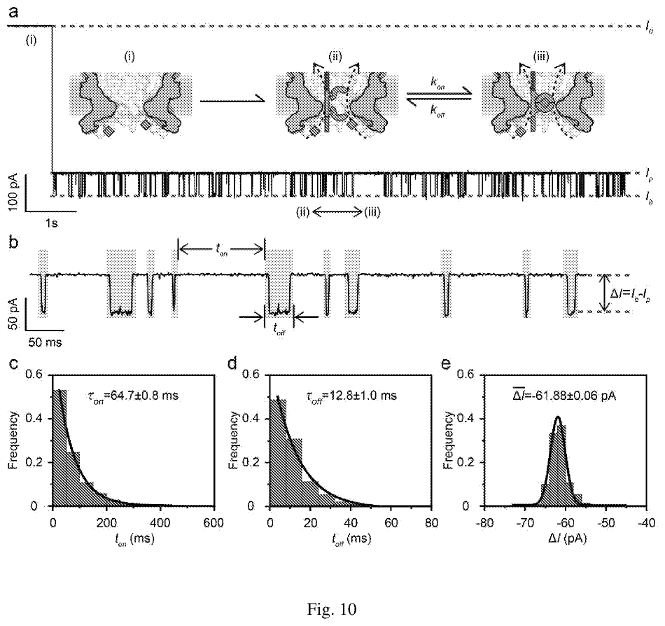

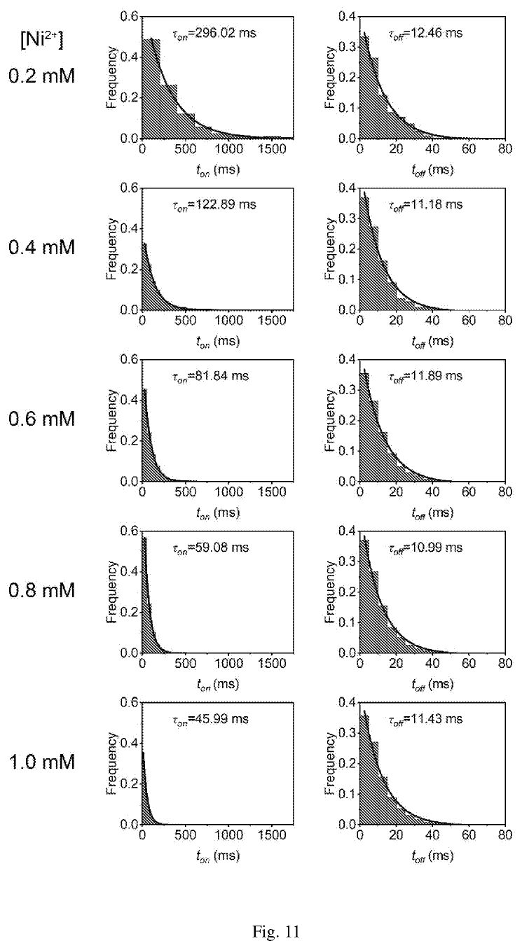

Core parameters that quantitatively describe any PNRSS event are summarized in FIG. 10, in which the dwell time t_{off} and the inter-event interval tₒₙ are defined. The blockage amplitude, ΔI is defined as I_{b}−Iₚ. The event scatter plot of ΔI vs t_{off} for measurement with 13G/14G and Ni²⁺ demonstrates a single population of events, in which ΔI=˜−60 pA (FIG. 1e), significantly larger than that produced by a monatomic ion, which is ˜2 pA in amplitude when observed with an α-HL²³. The mean dwell time, τ_{off} and the mean inter-event interval, τₒₙ are respectively derived as described in FIG. 10. The time histograms were summarized in FIG. 11, from which the corresponding τₒₙ and τ_{off} values were derived. As summarized in FIG. 1f, the reciprocal of the mean inter-event interval (1/τₒₙ) is proportional to [Ni²⁺ ], consistent with a single step bimolecular model, in which 1/τₒₙ=kₒₙ[Ni²⁺ ]. However, the mean dwell time, τ_{off} demonstrates a negligible dependence on [Ni²⁺ ], consistent with a unimolecular dissociation model, in which 1/τ_{off}=k_{off}. Thus, kₒₙ is determined as the slope of the fitted line of 1/τₒₙ vs. [Ni²⁺ ], and k_{off} is determined from the mean of the 1/τ_{off} value.

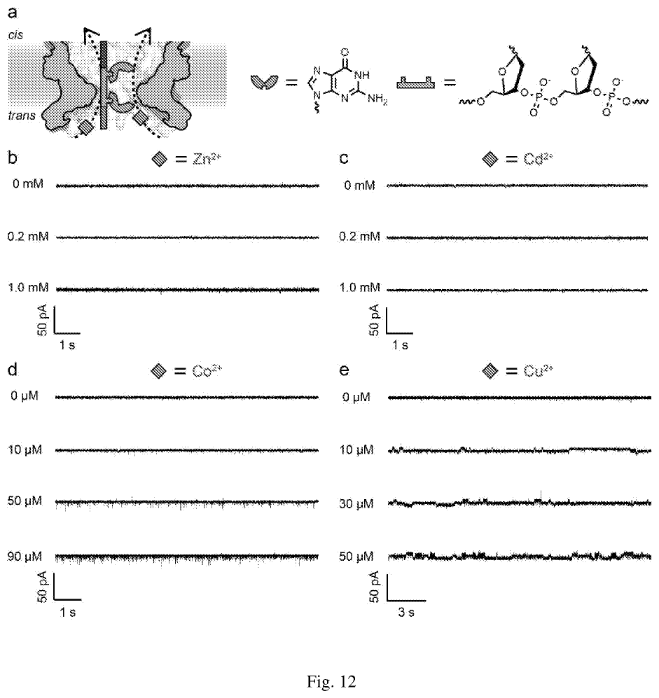

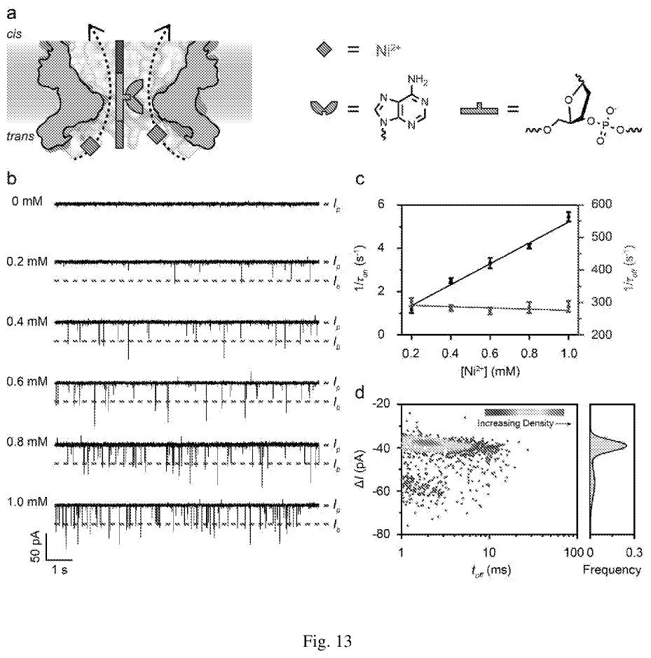

Other divalent ions such as Zn²⁺, Cd²⁺, Co²⁺ or Cu²⁺ were also tested with 13G/14G, which demonstrates a binding preference for Co²⁺ and Cu²⁺ over that of Zn²⁺ or Cd²⁺ (FIG. 12). Binding events from Ni²⁺, Co²⁺ or Cu²⁺ also dramatically differ from each other, indicating that single ion discrimination is feasible by PNRSS. Different PNRSS strands such as 14A, with a sole adenine as the fixed reactant or 14G, with guanine (Table 1), also have provided events of Ni²⁺ binding (FIG. 13-16). The binding characteristics here are however different from those observed with 13G/14G (Table 4), indicating that different chemical processes monitored by PNRSS were occurring. Although previously studied with NMR or infrared spectroscopy in ensembles^{36,37} direct single molecule observation of metal ion binding to DNA bases and their corresponding quantitative binding kinetics have not been reported before, to the best of our knowledge.

### Example 2 PNRSS with a 1,2,3-triazole

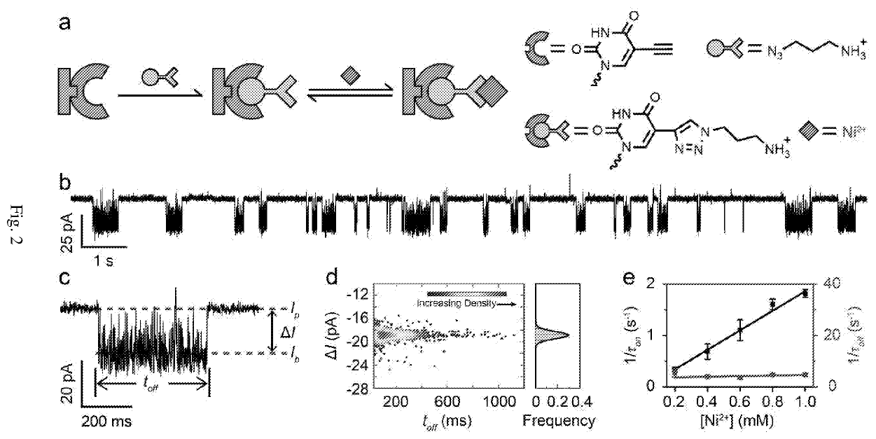

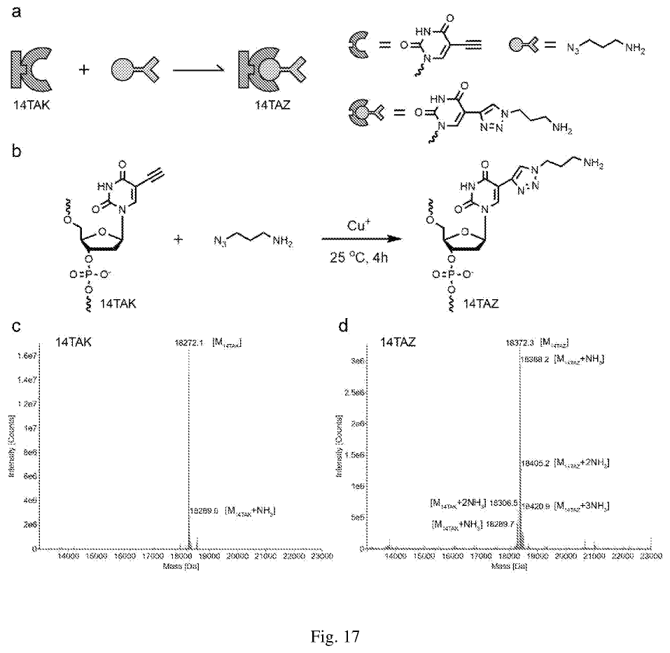

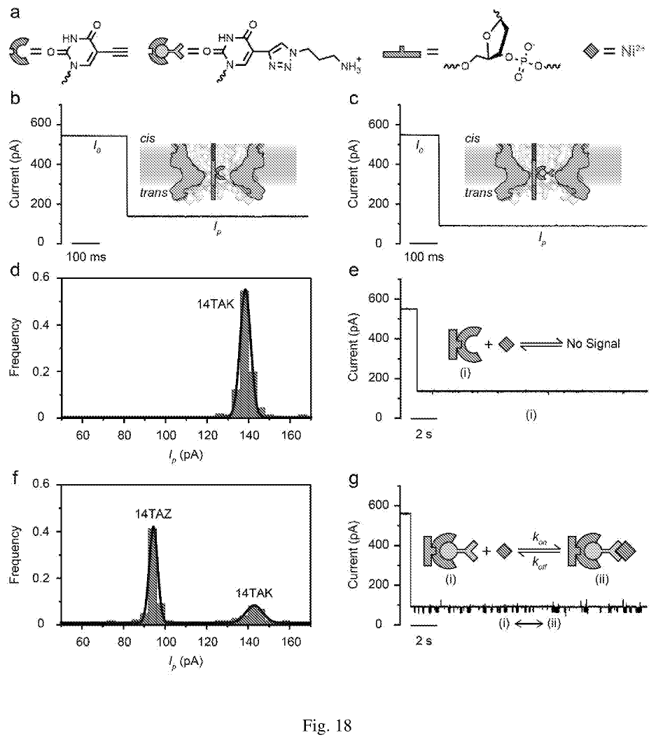

Beyond the above proof of concept, a much wider choice of artificial, functional DNA phosphoramadites provides a relatively unrestricted freedom in the design and synthesis of PNRSS strands, and thoroughly broadens the generality and the complexity of the downstream PNRSS measurements³⁸. 5-Ethynyl-dU-CE phosphoramidite (Glen Research, USA), a thymine derivative containing an alkyne, was used in the synthesis of the PNRSS strand 14TAK (Table 1, Methods). 14TAK contains a sole alkyne at site 14, to which any azides can be conjugated by a Huisgen copper (I)-catalyzed azide-alkyne 1,3-dipolar cycloaddition (CuAAC) reaction, a widely applied click chemistry reaction³⁹. 3-azidopropylamine, one of the simplest azides, was reacted with 14TAK (Table 1, FIG. 17). The product was characterized by mass spectrometry (FIG. 17) and single channel recording (FIG. 18, Table 5). The successful conjugation was confirmed, generating a new PNRSS strand referred to as 14TAZ (Table 1). From the reaction with CuAAC, a 1,2,3-triazole (TAZ) was generated at site 14 of 14TAZ (FIG. 2a).

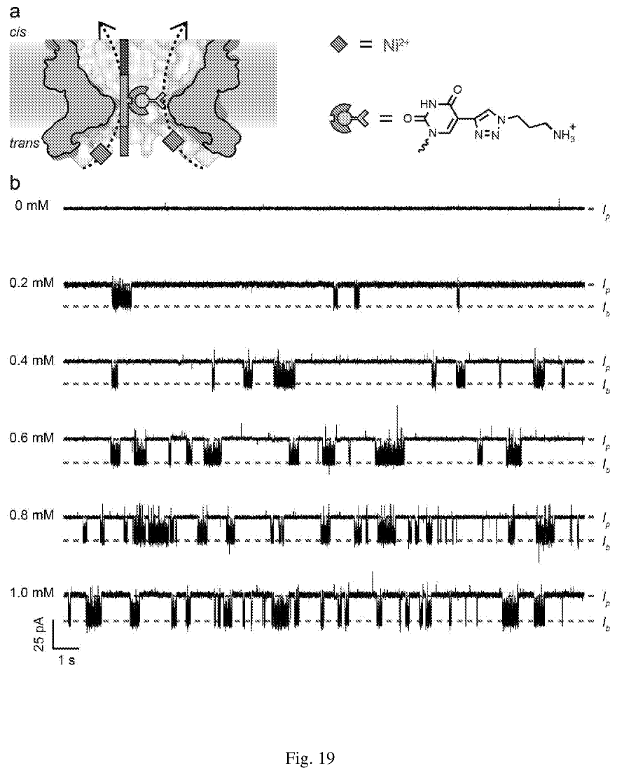

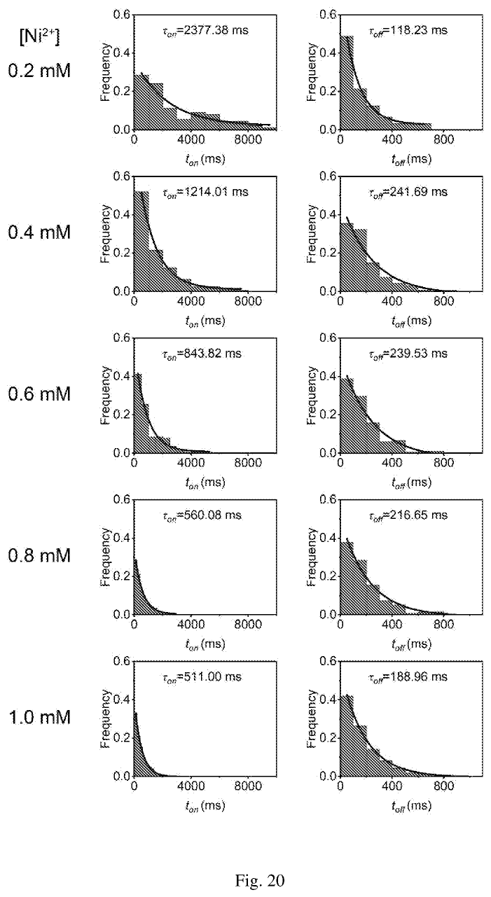

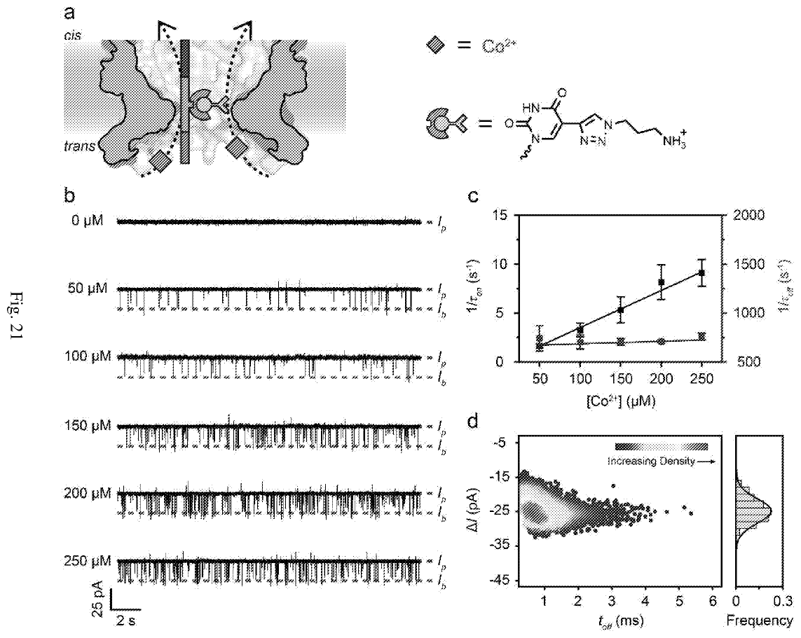

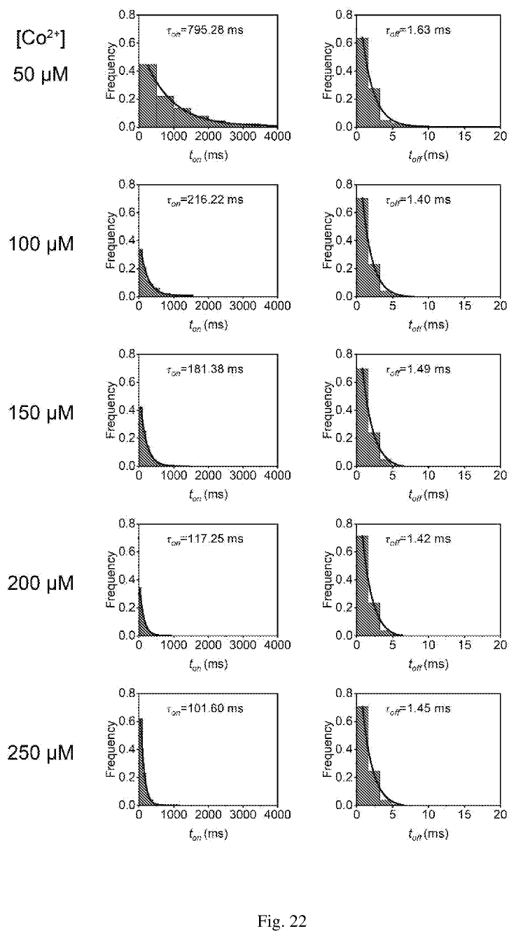

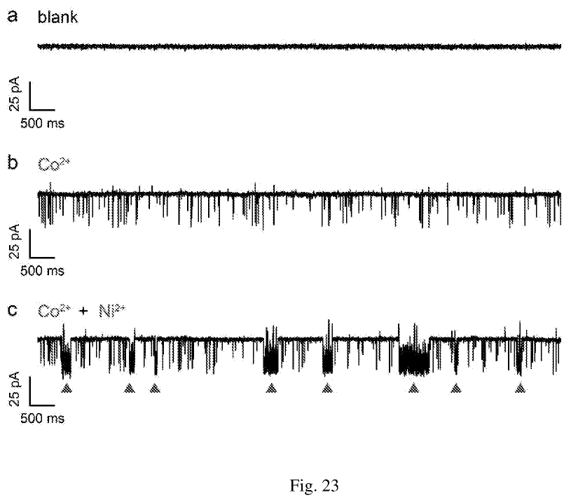

It has previously been reported that the N(2) or the N(3) atom of a TAZ can serve as an electron lone pair donor and act as a coordination site to bind metal ions⁴⁰. As a demonstration of the behaviour of a single molecule, a PNRSS measurement was carried out, in which the TAZ of the 14TAZ was applied as the fixed reactant. Ni²⁺, serving as a mobile reactant, was added to trans with a 1 mM final concentration. With a continuously applied +180 mV potential, successive appearance of binding events with a highly characteristic noise signal was observed (FIGS. 2b-2c, Video 2). A single distribution of events was reported in the scatter plot of ΔI vs t_{off} (FIG. 2d). The rate of event appearance was clearly increased by upregulating the Ni²⁺ concentration in trans from 0 to 1 mM (FIG. 19-20). The reciprocal of the mean inter-event interval (1/τₒₙ) is proportional to the Ni²⁺ concentration (FIG. 2e). However, the mean dwell time, τ_{off} demonstrates a negligible dependence on the [Ni²⁺ ]. Similar measurements were also performed with Co²⁺, in which the binding events appear as transient resistive pulses (FIG. 21-22), reporting a mean dwell time τ_{off} of 1.32 ms, much shorter than was observed with Ni²⁺, which was 220 ms (Table 6). The rate constants kₒₙ and k_{off} were respectively derived from results in FIGS. 2e and 21c. The equilibrium binding constant K_{b} was calculated as K_{b}=kₒₙ/k_{off}, in which Ni²⁺ demonstrates a significantly stronger binding affinity to a TAZ than Co²⁺ (Table 6). When Ni²⁺ and Co²⁺ were simultaneously examined, their binding events were unambiguously recognized (FIG. 23). The PNRSS strand 14TAK has thus demonstrated its generality as a template to introduce any azides. When reacted with the 3-azidopropylamine, the generated TAZ itself can serve as a fixed reactant, to which Ni²⁺ or Co²⁺ will bind.

### Example 3 PNRSS Performed with a Phenylboronic Acid

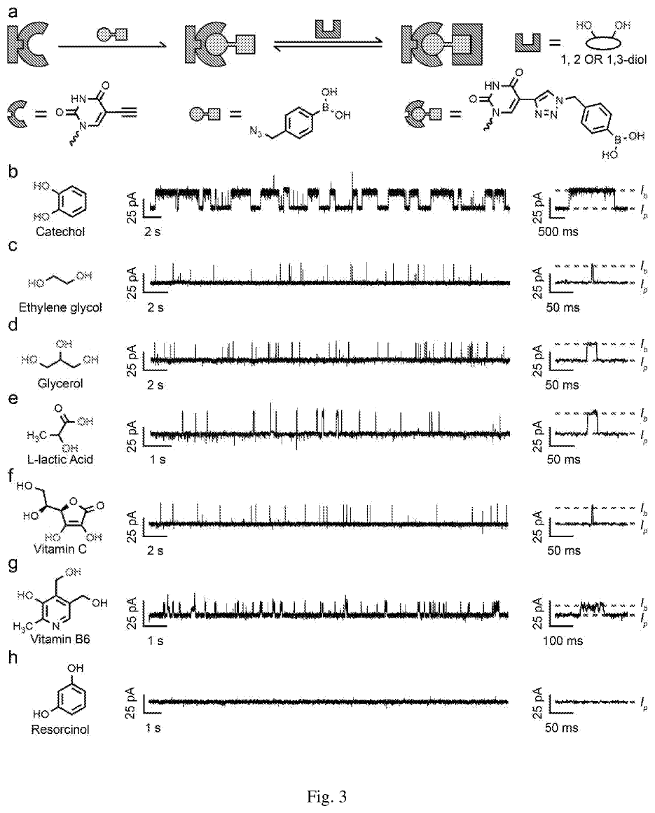

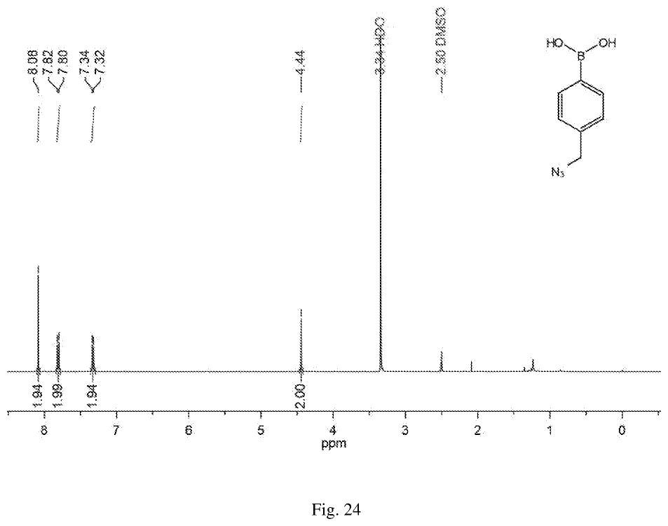

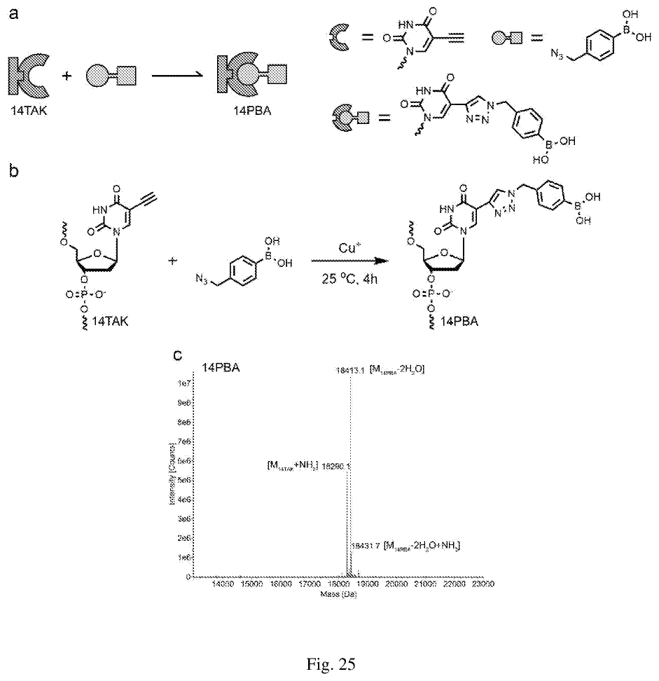

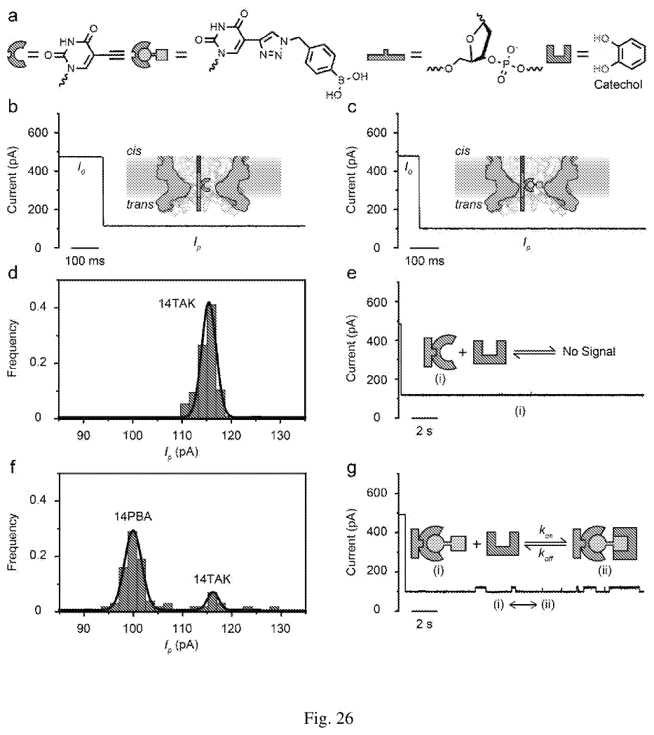

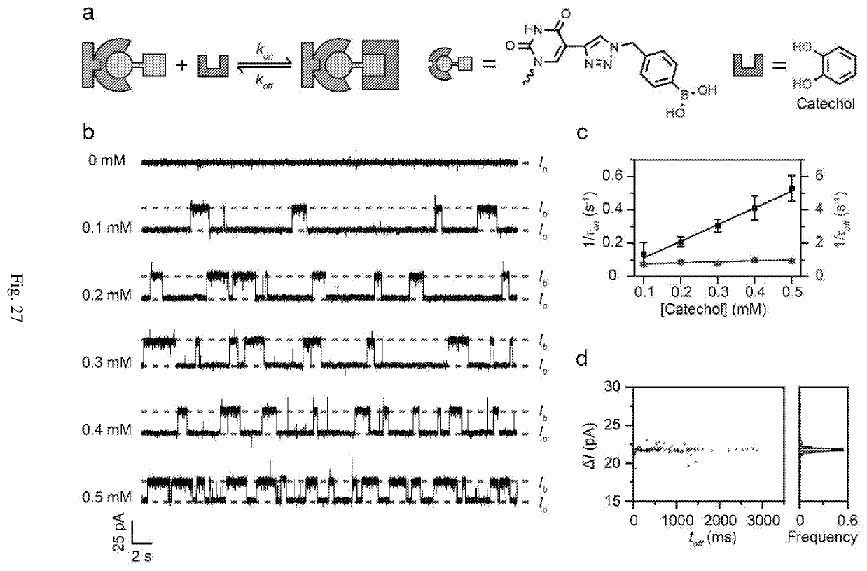

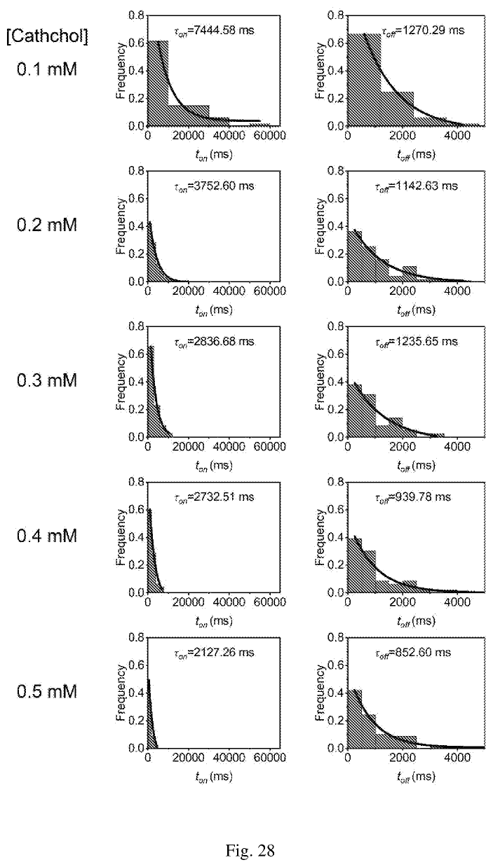

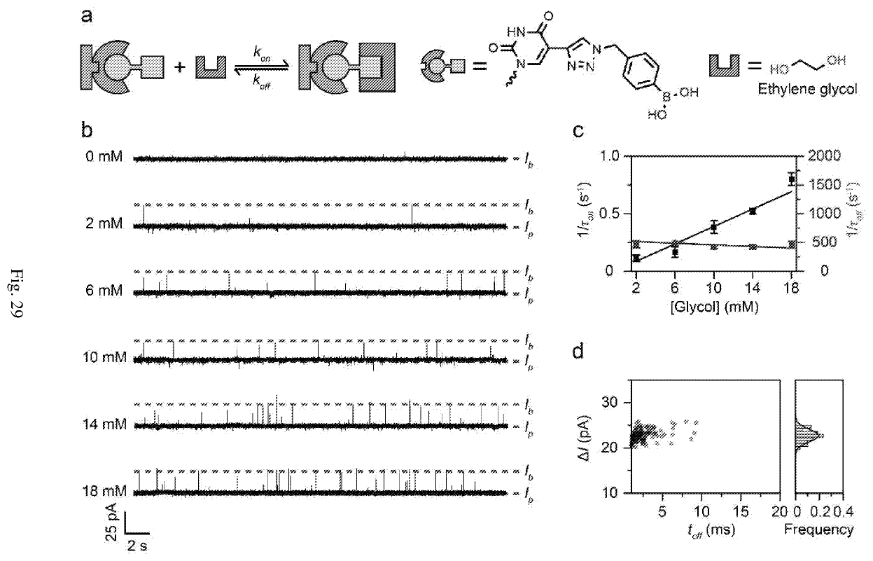

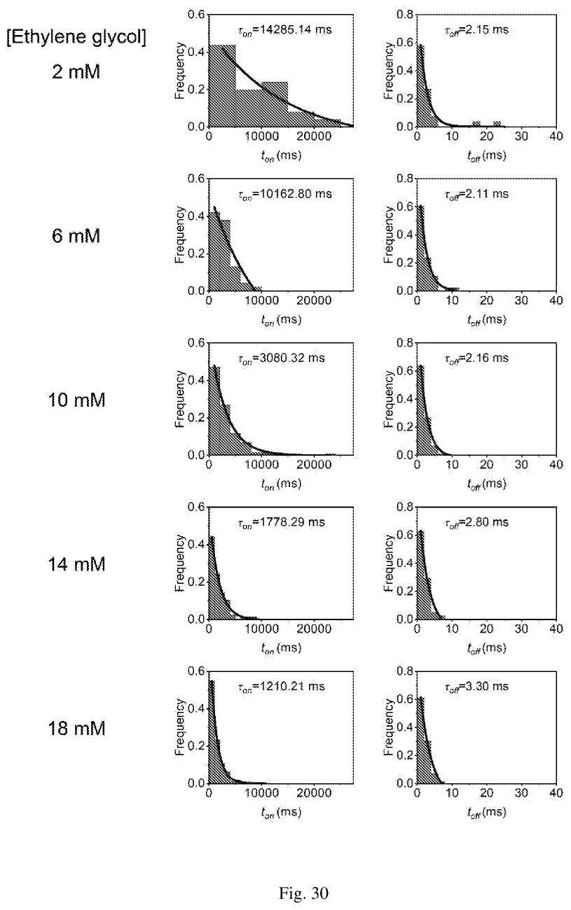

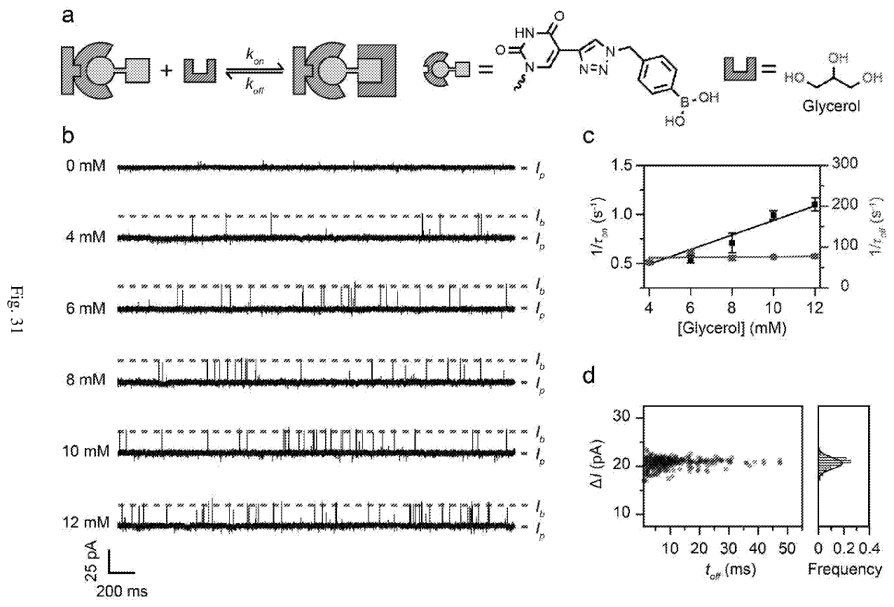

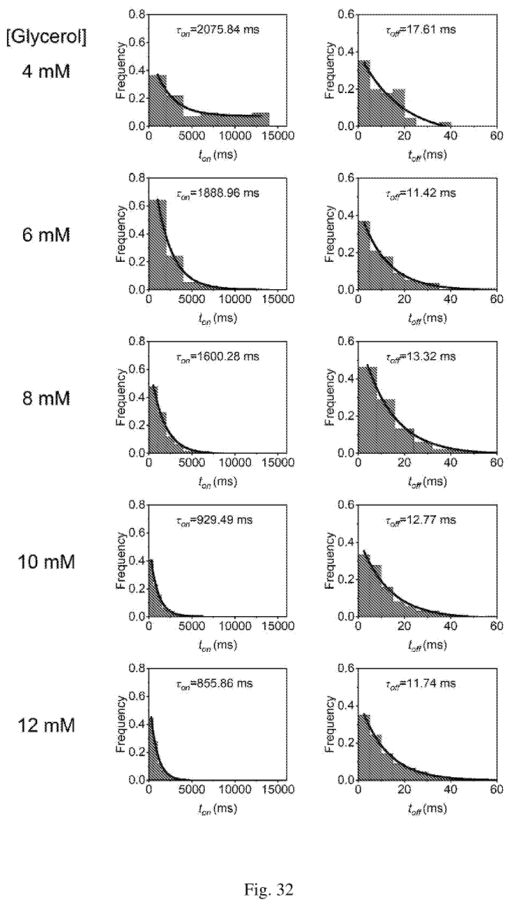

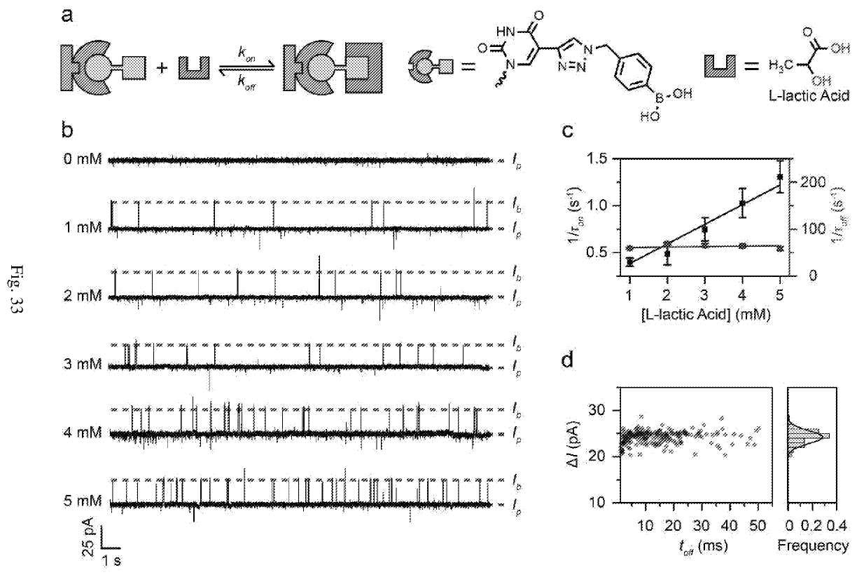

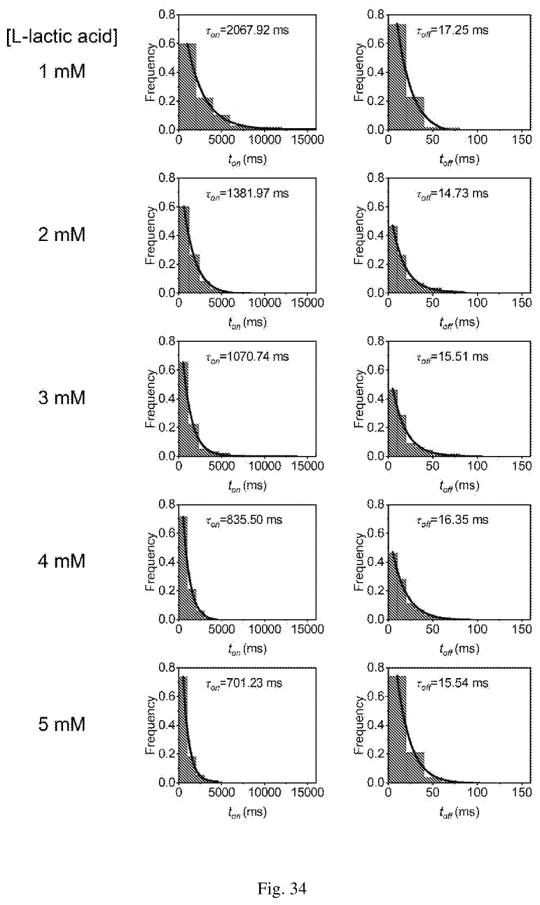

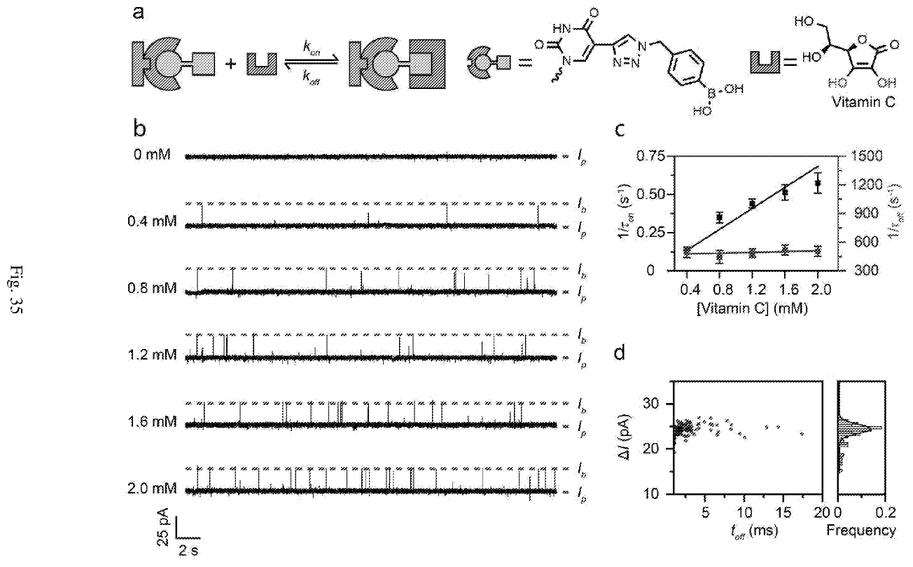

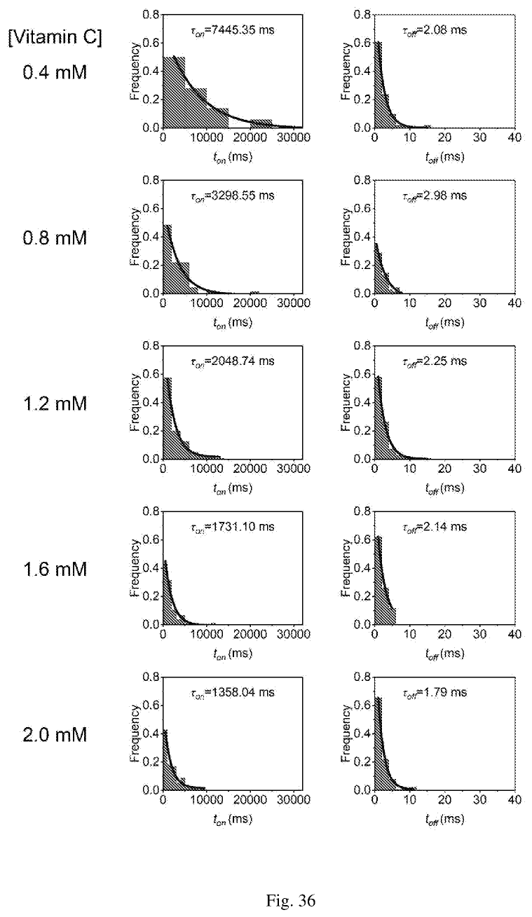

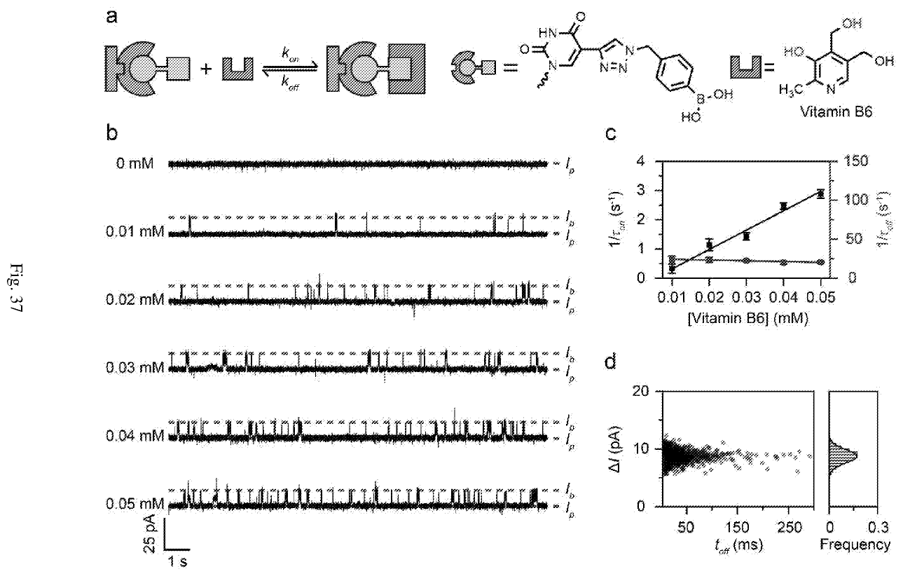

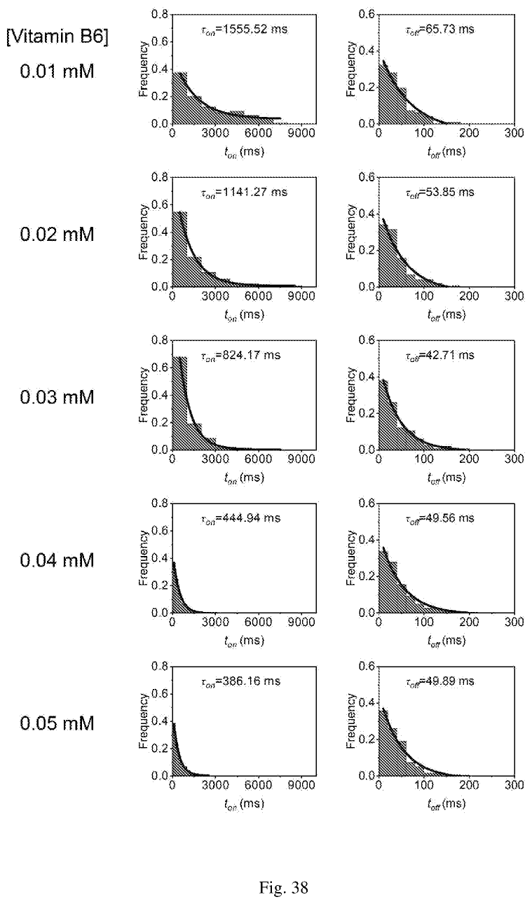

In most circumstances involving CuAAC, a TAZ is however treated as a linker, to which other functional modules can be attached⁴¹. Phenylboronic acid (PBA), the core component in sensors of saccharides or catecholamine⁴², reacts with compounds containing a 1,2-cis-diol or a 1,3-cis-diol moiety to form a five-membered or six-membered boronate ester (FIG. 3a) 43-4 To introduce a PBA to a PNRSS strand, 4-(azidomethyl)benzeneboronic acid was reacted with the PNRSS strand 14TAK by CuAAC (Methods, FIGS. 24-25). The product was further characterized by mass spectrometry (FIG. 25) and single channel recordings (FIG. 26, Table 7), which confirmed the success of the conjugation and generated a new PNRSS strand referred to as 14PBA (Table 1). To perform PNRSS, the PBA at site 14 of 14PBA serves as the fixed reactant. A +160 mV potential was continuously applied. Polyols such as catechol⁴⁴ (FIG. 3b, FIG. 27-28), ethylene glycol⁴⁵ (FIG. 3c, FIG. 29-30), glycerol⁴⁵ (FIG. 3d, FIG. 31-32), L-lactic acid⁴⁴ (FIG. 3e, FIG. 33-34), vitamin C⁴⁶ (FIG. 3f, FIG. 35-36) or vitamin B6⁴⁷ (FIG. 3g, FIG. 37-38), serve as the mobile reactant and were individually added to trans at a desired concentration. Though the binding characteristics and kinetics differ (Tables 8-9), all the aforementioned reactants reported detectable binding to a PBA. In contrast, when probed by 14TAK, no binding events were observed, confirming that the events registered the result of binding to the PBA (FIG. 26e). Resorcinol, which is structural incompatible with reaction with a PBA, failed to produce any binding events (FIG. 3h), further confirming the reaction mechanism. All observed reactions discussed in FIGS. 3b-3g report positive events (I_{b}>Iₚ). This is counterintuitive because a bound molecule, which occupies more space in the nanopore lumen, is expected to produce negative events (I_{b}<Iₚ). A proposed mechanism is that binding of any analyte described above to a PBA results in the generation of an anionic boronic ester, as reported in several previous literatures^{48,49}. This generated negative charge may generally enhance the ionic flow through the pore. An evidence to approve that introduction of negative charges to the pore constriction would enhance the pore conductance is that the wildtype MspA which has more negative charges on the pore constriction than that of M2 MspA, is significantly more conducting than M2 MspA in the open pore state⁷. On the other side, the overall size of the bound analyte may contribute to the reduction of the ionic flow. Thus, the overall contribution of a bound analyte to the blockage amplitude may be positive, especially when the studied analyte is a small molecule. A systematic study using quantum chemistry and molecular dynamics simulations may be carried out to further quantify this phenomenon but in a separate follow up study.

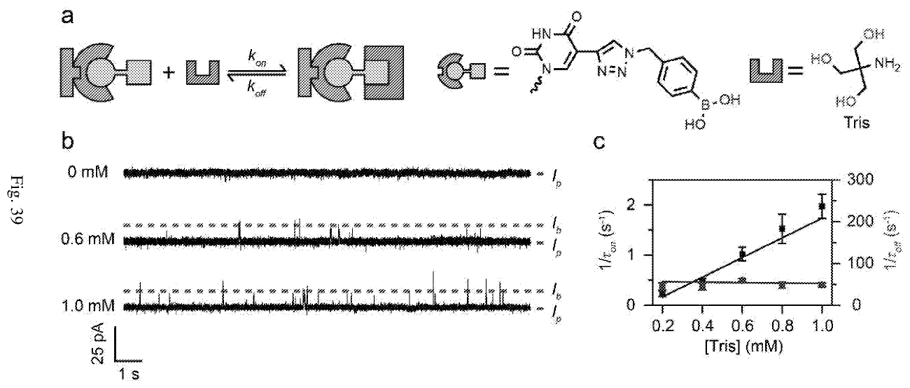

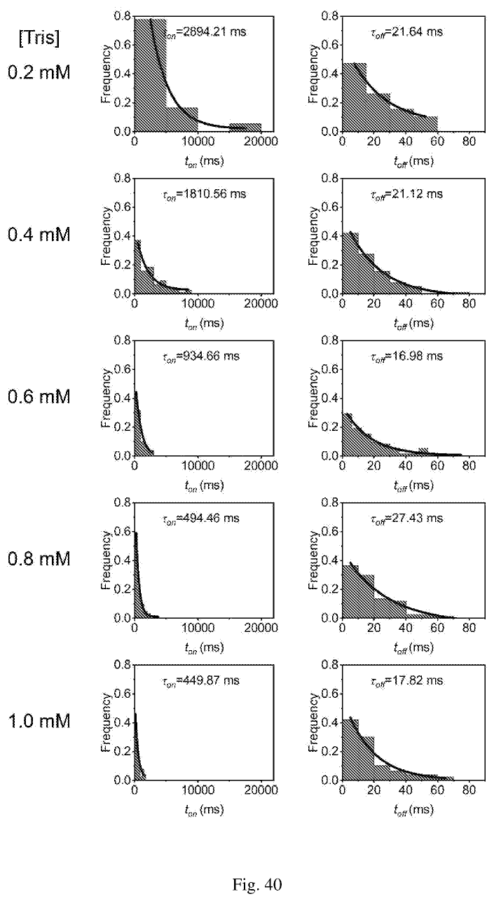

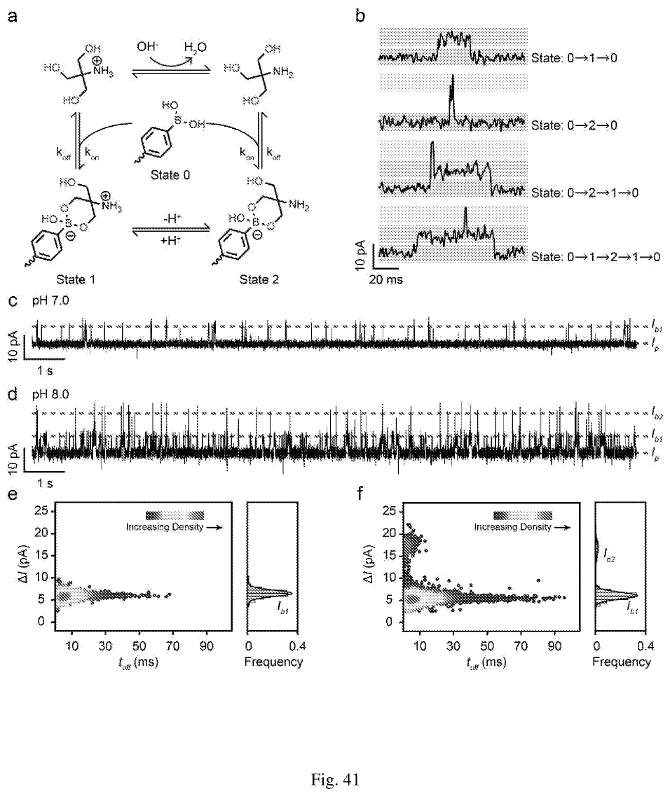

A core advantage of nanopore based single molecule chemistry is that transient appearance of chemical intermediates can be probed, at a μs resolution s. Tris(hydroxymethyl)-aminoethane (tris) is a widely used buffer reagent, whose conjugate acid has a pKa of ˜8.3⁵¹. When dissolved in an aqueous solution at a pH near to its pKa, protonated or deprotonated forms of tris would both exist in significant proportions. In either form, a tris molecule, as a polyol, can react with a PBA and chemical intermediates resulting from its protonation or deprotonation may be observed. Though tris is easily accessible and widely used, tris binding to a PBA appears not to have been investigated to date. To demonstrate the direct observation of chemical intermediates with PNRSS, PBA and tris were applied as the fixed and the mobile reactant respectively (FIGS. 39-40). At pH 7.0, binding of tris to PBA results in positive events, reaching the level I_{b1}. At pH 8.0 however, an extra binding level (I_{b2}) was observed, in addition to I_{b1}. Dynamic switching between Iₚ, I_{b1} and I_{b2} was also observed, where I_{b1} and I_{b2} represent the protonated and the deprotonated tris respectively, when bound to a PBA (FIG. 41). The observation that the deprotonated state which is more negatively charged reports a higher blockage state (I_{b2}>I_{b1}) is also consistent with our previous speculation that more negative charges generally enhances the ionic flow in this measurement configuration. Demonstrated with this simple example, direct observation of chemical intermediates enables an understanding of transient chemical processes. When properly designed, the appearance of chemical intermediates also helps to discriminate between subtly different chemical compounds. The above demonstration also suggests that a tris buffer should be avoided in any PBA-based polyol sensing assay by PNRSS so as to avoid interferences.

### Example 4 Repetitive Observation of Irreversible Reactions

According to the previously reported nanopore based single molecule chemistry measurement⁵², only a sole reactive site was permanently fixed in the pore lumen, meaning that any irreversible chemical reaction at this site would immediately terminate the production of any new information of chemical processes. Though rarely discussed, this technical restriction limits nanopore single molecule chemistry studies to only reversible reactions. However, with PNRSS, the strand containing the fixed reactant is chemically separated from the pore. Even if it is irreversibly reacted, the whole PNRSS strand can be voltage rejected and reloaded, re-initiating a new cycle of measurement.

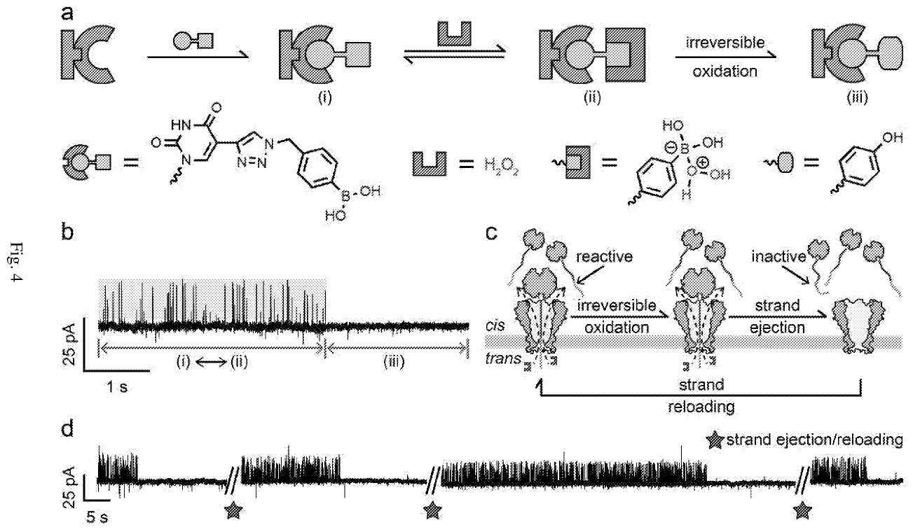

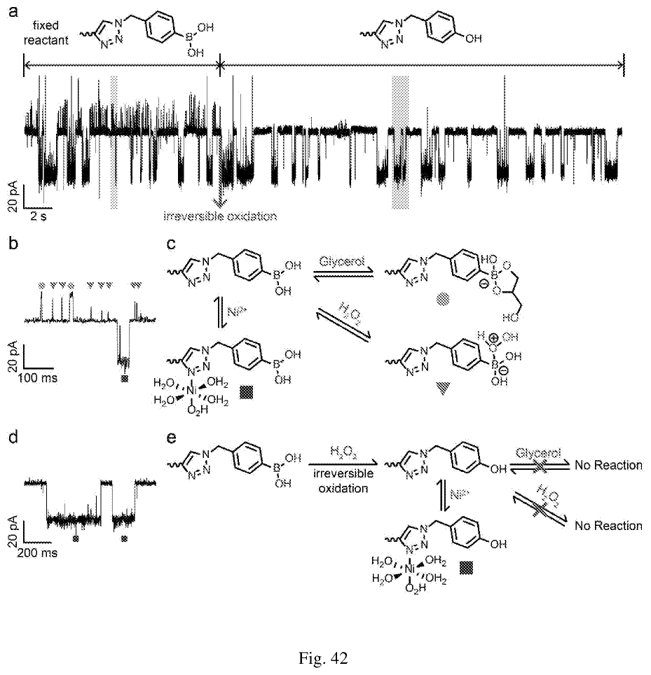

To demonstrate this, hydrogen peroxide (H₂O₂), a strong oxidant capable of irreversibly oxidizing a PBA to a phenol⁵³, was used as the mobile reactant (FIG. 4a) and a PBA was applied as the fixed reactant. During PNRSS, H₂O₂ initially reacts reversibly with the PBA, generating positive going spiky events, likely resulting from an intermediate state prior to the production of a phenol (FIG. 4a ii) as proposed in literatures^{54,55}. However, the boron atom is not yet removed at this stage. Later, these spiky events suddenly disappear, giving a fluctuation-free baseline (FIG. 4b). In this state, this strand can still react with Ni²⁺, indicating that the 1,2,3-triazole linker is still present. However, it can no longer react with any polyol or H₂O₂, indicating that the boronic acid group has been lost, resulted from being irreversibly oxidized to a phenol (FIG. 42). However, a new measurement cycle can be re-initiated by a voltage protocol (FIG. 4c) so that an irreversible single molecule reaction can now be repetitively monitored, acknowledging this unique property of PNRSS (FIG. 4d, Video 3).

### Example 5 Discrimination of Epinephrine, Norepinephrine and Isoprenaline

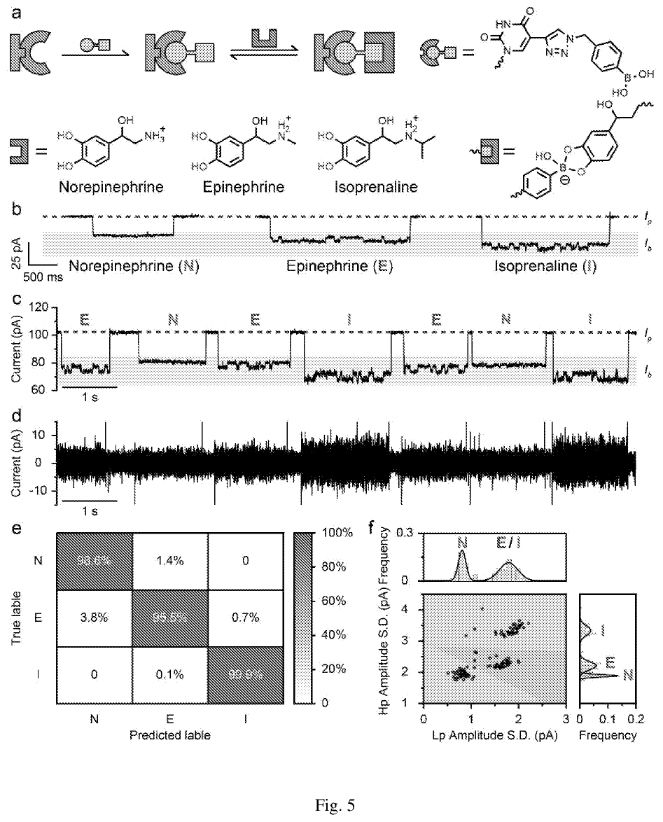

Besides the measurement of binding kinetics, rich information of chemical reactions monitored by PNRSS is also useful in recognition of single molecules. Norepinephrine, epinephrine and isoprenaline are catecholamine derivatives⁵⁶. Norepinephrine and epinephrine are both natural hormones and neurotransmitters and can be used medically⁵⁷. Isoprenaline is a sympathomimetic beta-adrenergic agonist medication⁵⁸. Specifically, norepinephrine acts mostly on alpha receptors and serves to maintain blood pressure while epinephrine less specifically stimulates both alpha and beta receptors and serves to relax the breathing tubes and to regulate blood flow, heart rate and glycogen metabolism. Isoprenaline, however, is used mainly in the treatment of bradycardia, heart block and asthma. These functional differences result from subtle variations in their chemical structures. They all however contain a 1,2-benzenediol moiety which reacts with a PBA (FIG. 5a).

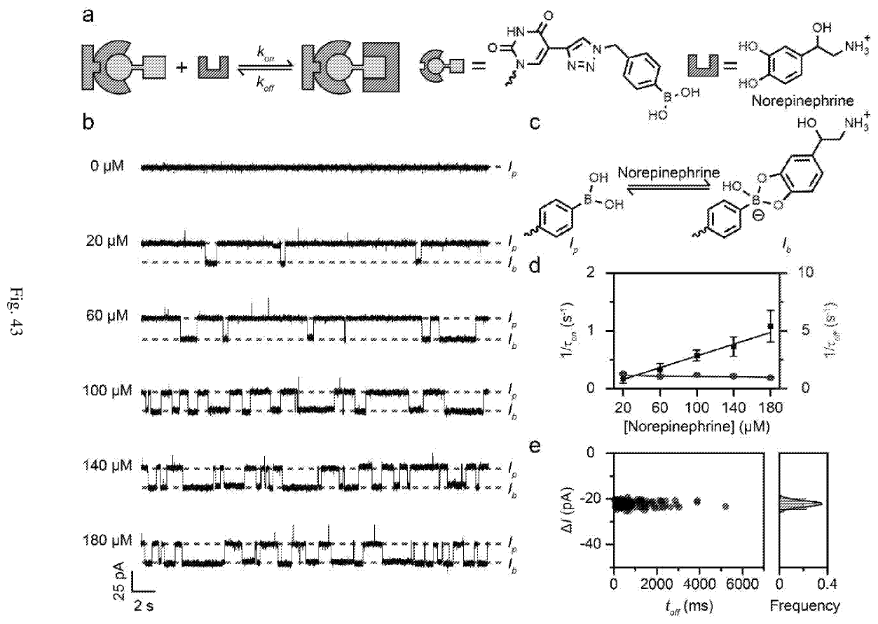

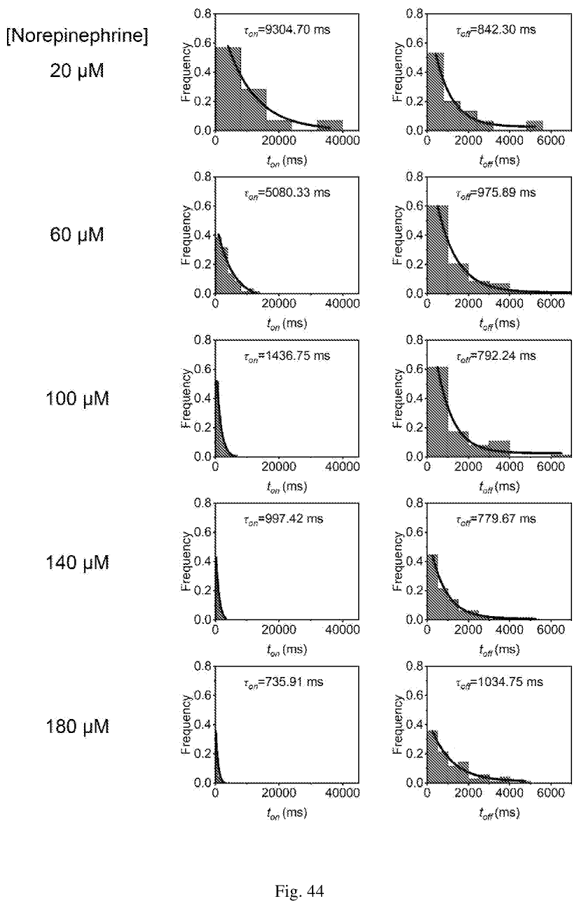

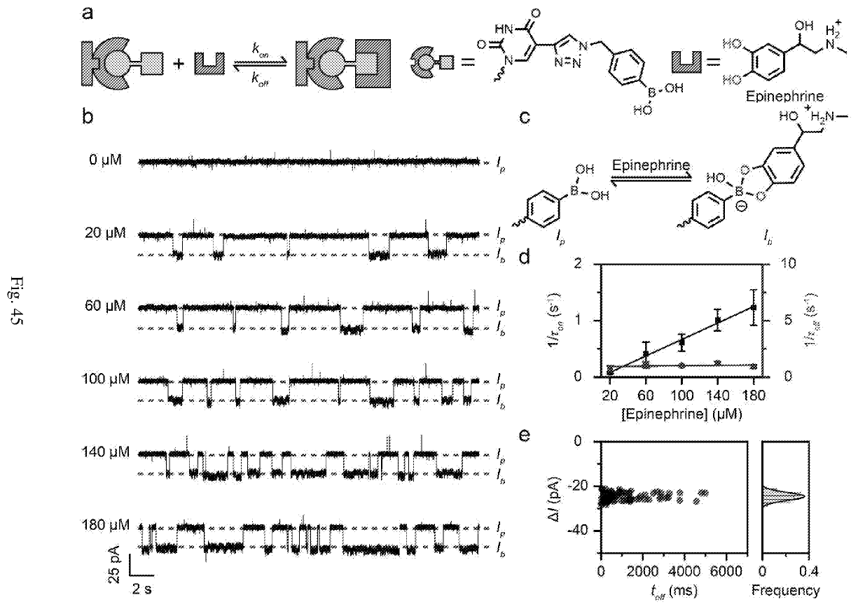

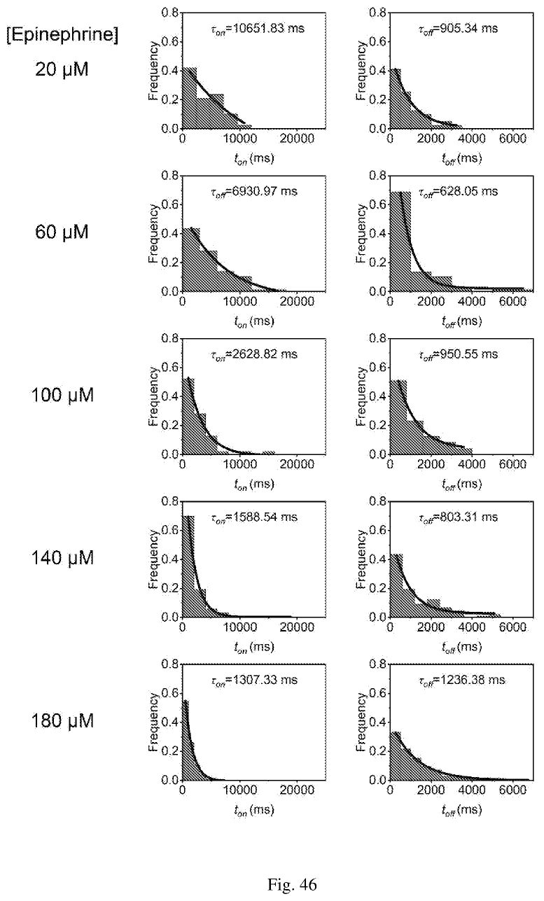

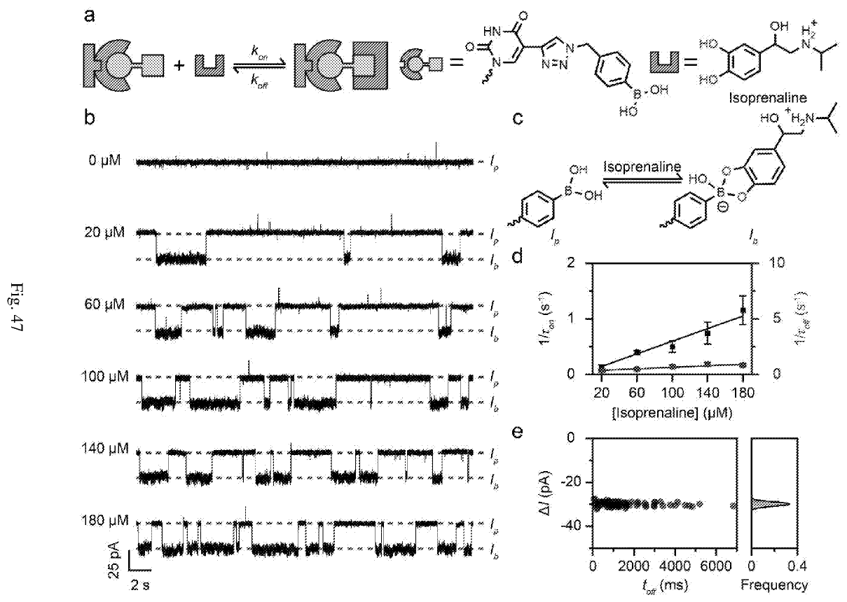

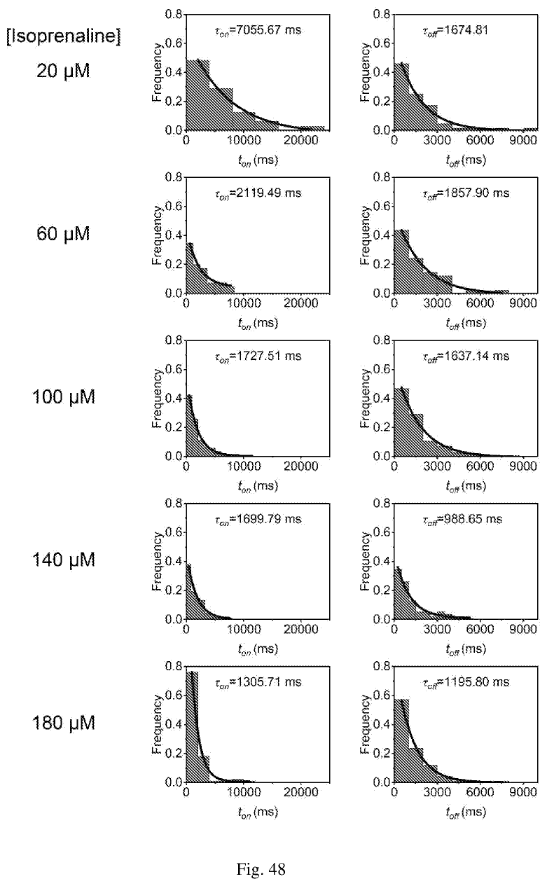

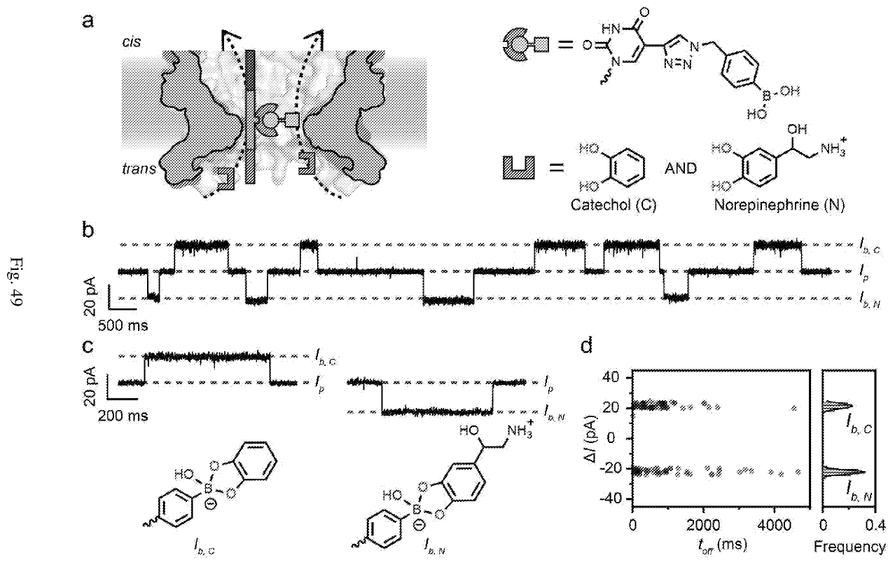

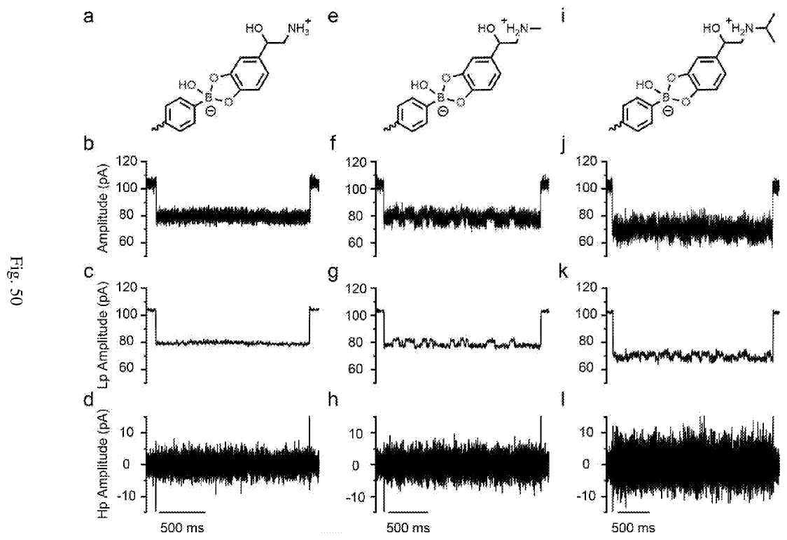

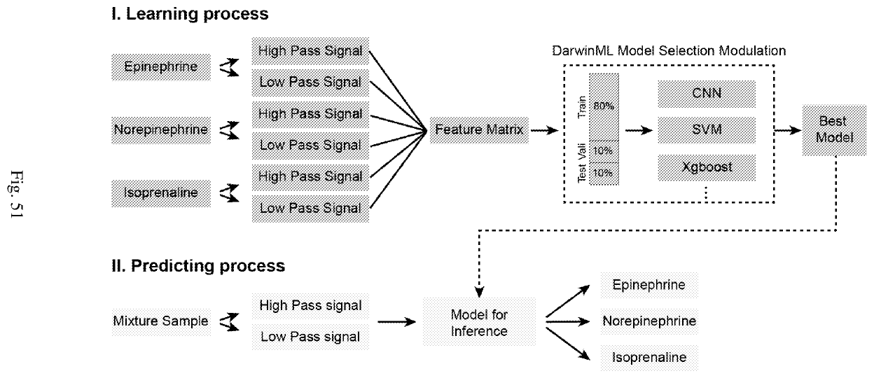

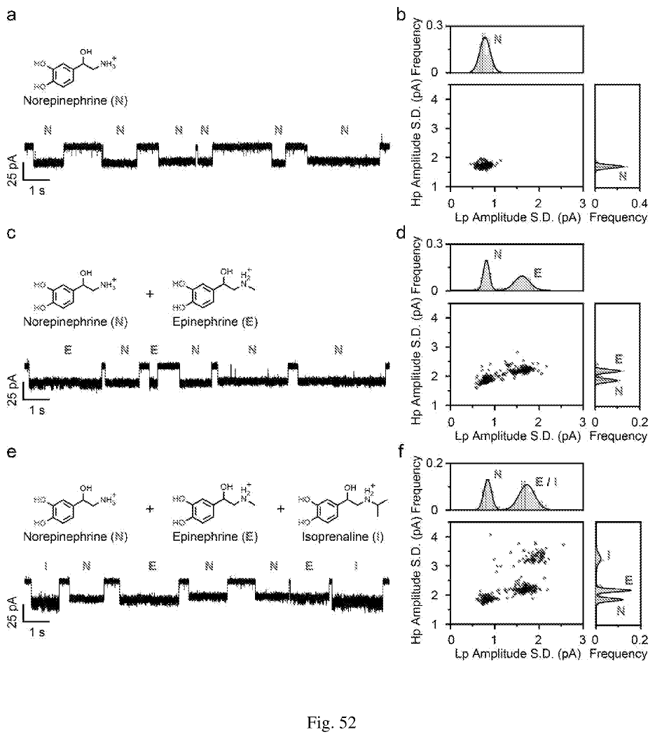

PNRSS measurements of these compounds were carried out with 14PBA. Norepinephrine (FIG. 43-44), epinephrine (FIG. 45-46) or isoprenaline (FIG. 47-48) were respectively added to trans, reaching desired final concentrations. With a +160 mV potential continuously applied, all three compounds report negative proceeding binding events (I_{b}<Iₚ), dramatically different from that of the catechol which reports positive proceeding events (I_{b}>Iₚ). It is speculated that norepinephrine, epinephrine and isoprenaline, which are all cationic, may have compensated the effect of enhanced ionic flow due to the generation of the anionic boronic ester. This difference was more clearly demonstrated when catechol and norepinephrine were simultaneously sensed (FIG. 49, Video 4). Though their binding affinities are similar (Table 10), binding events caused by norepinephrine, epinephrine or isoprenaline are however distinguishable by their distinct binding characteristics, including ΔI, τ_{off} or noise levels. The acquired traces were first split by frequency into a low pass and a high pass portion, performed by a Butterworth filter with a 100 Hz cut off frequency (FIG. 50). The low pass portion of events caused by norepinephrine binding reports ΔI of a smaller amplitude. The bound state is also free of any additional fluctuations. In contrast, events caused by epinephrine or isoprenaline binding result in ΔI of a larger amplitude, in addition to which secondary telegraphic fluctuations were observed (FIG. 5b). Events caused by epinephrine or isoprenaline can however be more clearly separated from their high pass portion, in which binding of isoprenaline produces a high frequency noise with a much larger amplitude. These differences in binding characteristics are more clearly demonstrated in the simultaneous sensing assay (FIGS. 5c and 5d, Video 5). The events caused by binding of different catecholamines were efficiently distinguished by the standard deviation (S.D.) value of the low pass and the high pass portion. For automatic event identification, these two parameters were applied to build a machine learning algorithm (Methods, FIG. 51). Briefly, the algorithm was developed based on the support vector classification (SVC) model, a machine learning method for data classification⁵⁹. A total of 1455 events acquired with epinephrine, norepinephrine or isoprenaline as the sole analyte were applied to finalize the model. According to the confusion matrix results, events caused by isoprenaline were reported with an impressive 99.9% accuracy. Those caused by epinephrine or norepinephrine were reported with a satisfactory 95.5% or 98.6% accuracy (FIG. 5e). When simultaneously sensed, events from a continuously recorded trace 15 min in length were extracted, and the low pass and the high pass standard deviation values were presented (FIG. 5f). Three event populations, from binding of norepinephrine, epinephrine or isoprenaline, were clearly separated (FIG. 52). A decision boundary plot generated by the machine learning algorithm was placed above the scatter plot to assist event recognition. The above demonstration with the three catecholamines has provided concrete evidence that the wealth of information generated by chemical reactions can facilitate single molecule recognition. Though the reaction between a PBA and catecholamine is well understood^{60,61}, it is the first single molecule chemistry study on the topic to be studied, and was facilitated by PNRSS. Different from a previous report of neuron transmitter sensing by nanopore⁶², in which only a weak amplitude of a 0.9 and a 1.1 pA were reported from epinephrine and norepinephrine respectively binding to an engineered α-HL, this approach with PNRSS applied a different chemical reaction and a conical pore, resulting in richer sensing information and a much larger event amplitude (˜21-32 pA), clearly distinguishing three catecholamines (Table 11). Frequency split analysis assisted by machine learning has further boosted the sensing performance. Distinct from the strategy of fluorescence probe design applied in ensembles, in which chemical synthesis of complicated probe structures are necessary to distinguish chemically similar catecholamines^{63,64}, the strategy by PNRSS requires only a sole PBA to distinguish three catecholamines. Other catecholamines, such as dopamine and L-3,4-dihydroxyphenylalanine (L-DOPA), which are precursors of norepinephrine and epinephrine⁶⁵, were also studied by this system. These results however will be reported separately in a follow up study.

### Example 6 Discrimination of Remdesivir and Remdesivir Triphosphate Metabolite

A variety of compounds based on nucleoside analogues have been synthesized, screened and clinically tested in the development of anti-viral drugs⁶⁶. Remdesivir, a specific nucleoside analogue and an investigational anti-viral drug⁶⁷, has been reported to be effective in treating conditions caused by 2019 coronavirus (COVID-19), the agent responsible for the current pandemic that has caused a global crisis⁶⁸⁻⁷⁰. Remdesivir, a prodrug, is metabolically converted in cells into its active triphosphate form, which acts to block the RNA-dependent RNA polymerase (RdRp), precluding the virus from further replication⁷¹.

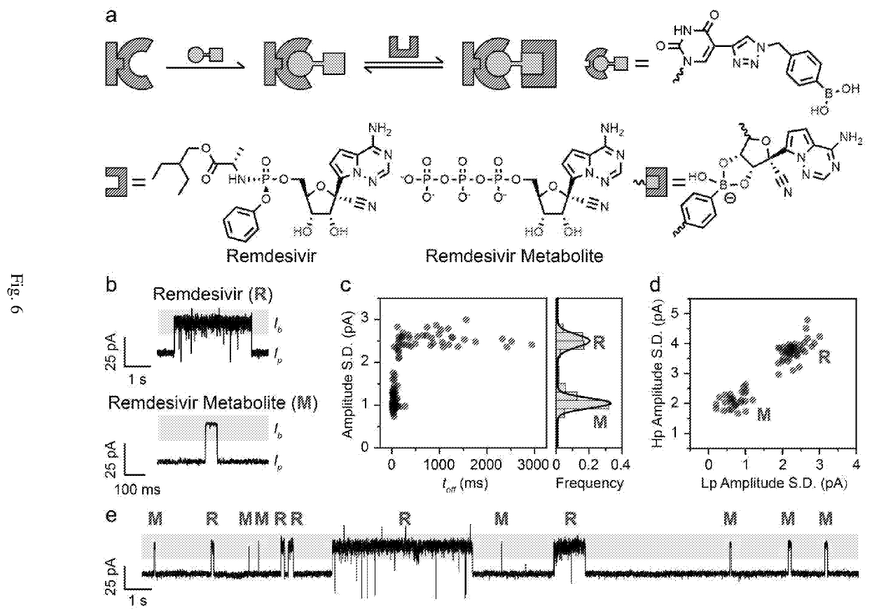

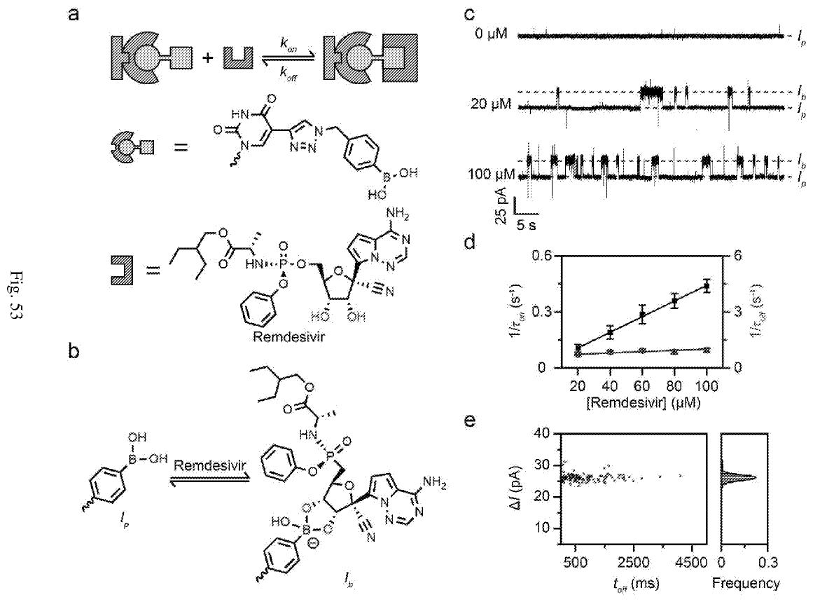

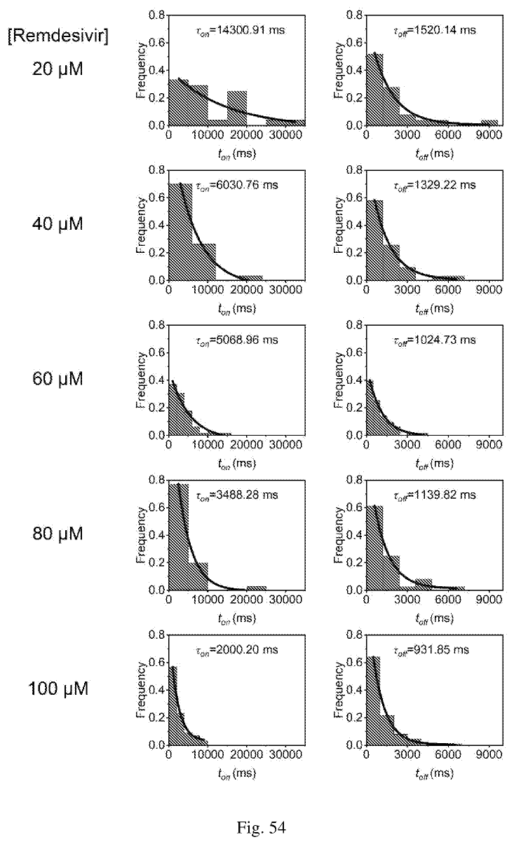

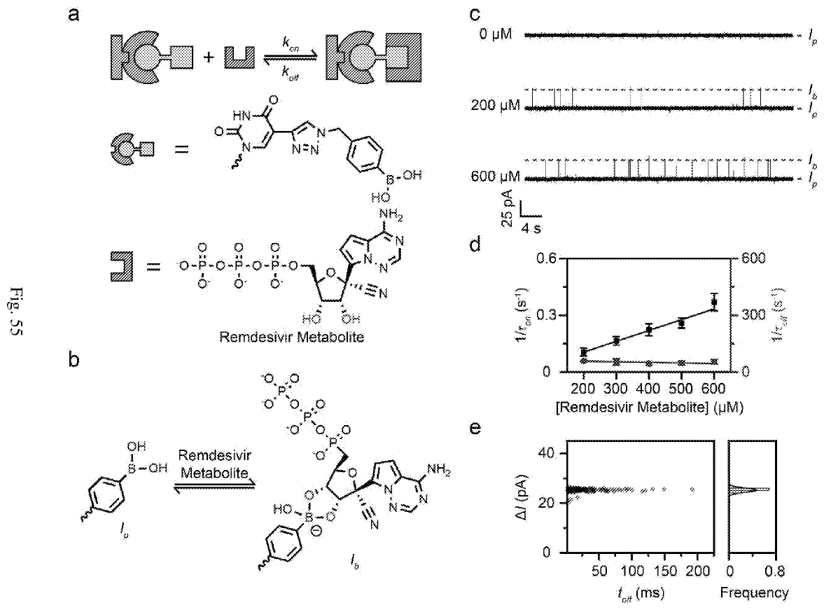

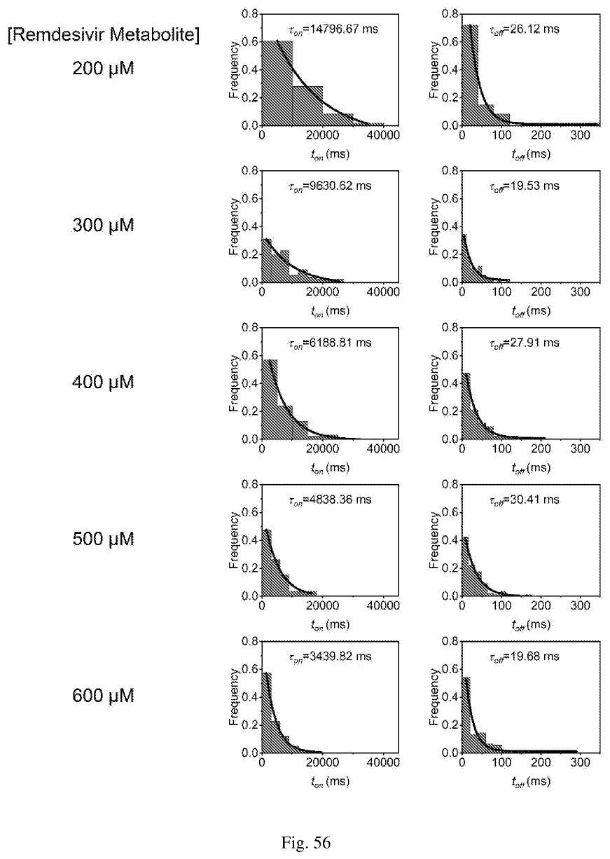

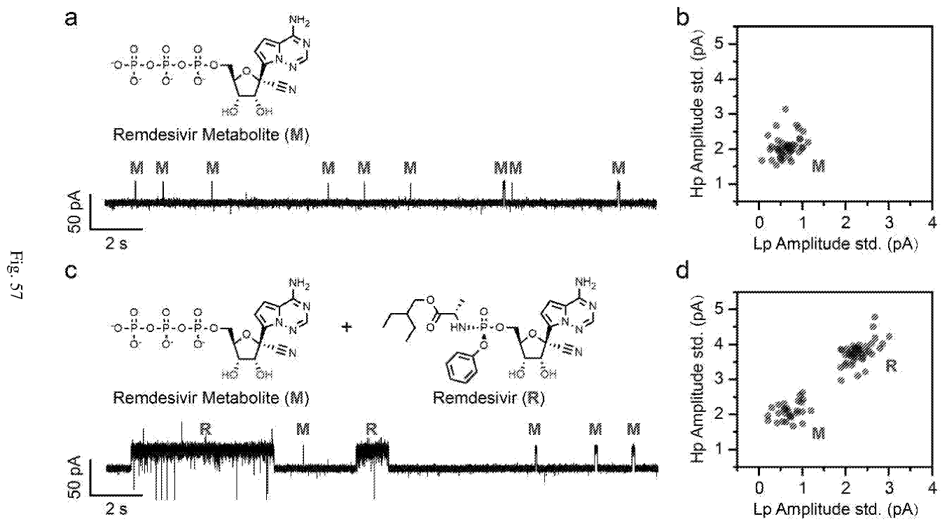

Remdesivir and its triphosphate metabolite both contain a ribose moiety, which reacts with PBA⁷² (FIG. 6a). The corresponding PNRSS assay was designed with 14PBA placed in cis. Remdesivir (FIG. 53-54) or remdesivir triphosphate (FIG. 55-56) were treated separately as the mobile reactant and respectively added to trans at the desired concentrations. A +160 mV potential was continuously applied. During PNRSS, both remdesivir and remdesivir triphosphate report positive proceeding events. However, events from remdesivir are long-resident, and intensive fluctuations were also observed while events of remdesivir triphosphate are much shorter resident and negligible level fluctuations were observed (FIG. 6b, Table 12-13). These distinct event characteristics are clearly demonstrated when the S.D. of the blockage level is plotted against the even dwell time (FIG. 6c). Distinction of the events is also demonstrated by the scatter plot of the high pass and the low pass standard deviation values, from which two populations are separated unambiguously (FIG. 6d, FIG. 57). A continuous trace performed with both reagents added is demonstrated in FIG. 6e, in which differences between both event types can be clearly recognized (Video 6).

Though there are conflicting conclusions on the therapeutic effect of remdesvir in treating COVID-19^{73,74}, the above demonstration has nevertheless expanded the types of analytes that can be investigated by PNRSS. Though the chemical reaction between PBA and nucleoside analogues has been previously investigated⁷², binding between remdesvir and its derivative to a PBA has not been studied to date. With PNRSS, their single molecule binding kinetics are determined, in a nano-confined space (Table 13). Direct distinguishing of remdesvir and its metabolite is also achieved. Though not demonstrated in this paper, other nucleoside analogues such as Galidesvir⁷⁵, Ribavirin⁷⁶ or Favipiravir-RTP⁷⁷ may in principle be recognized by a similar PNRSS assay. These demonstrations may inspire pharmacokinetics or drug screening applications and may be useful in the current pandemic.

### Example 7

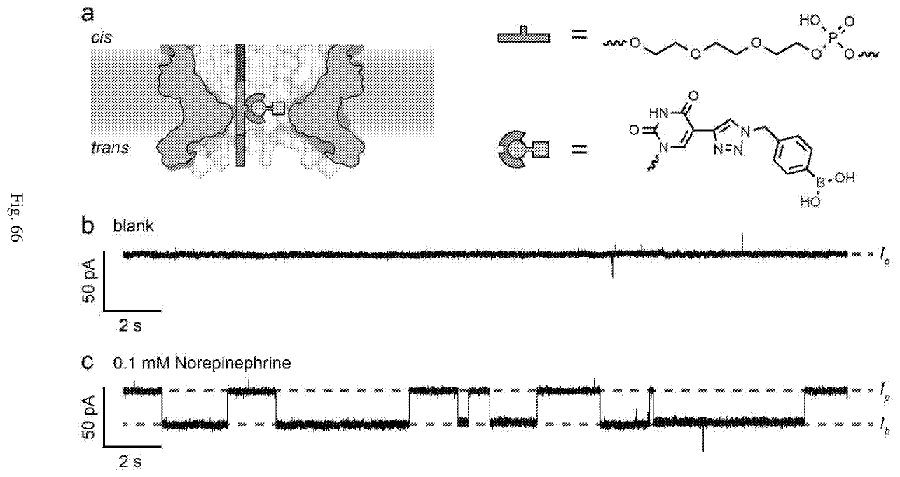

A PNRSS strand can be composed of any synthetic polymer such as nucleic acid, peptide, polysaccharide or combinations thereof, but to study a wider variety of single molecule reactions, the composition of the PNRSS strand should be arbitrarily programmable. As shown in the picture above, we designed a PNRSS strand consists of oligonucleotides and polymer (FIG. 66a). The polymer unit was formed by the polymerization of three molecules of ethylene glycol. In addition, this strand contains a sole PBA, capable of binding norepinephrine. In this case, norepinephrine report negative proceeding binding events at +160 mV (FIGS. 66b and c). In summary, PNRSS strand can be composed of polymers in PNRSS technology.

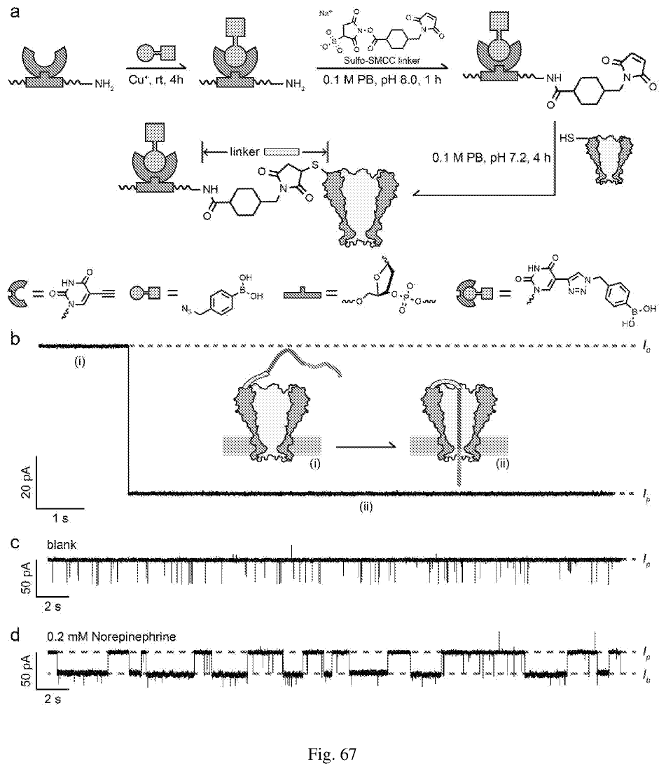

In PNRSS measurement, a streptavidin-tethered PNRSS strand is electrophoretically docked, remaining fully stretched in the PNRSS pore. However, as shown in FIG. 67, we demonstrate another PNRSS method without avidins named fixed-PNRSS (fPNRSS). In this case, we designed a PNRSS strand conjugated to MspA protein, which eliminates the need for PNRSS chains in solution. This PNRSS strand can be electrophoretically docked, remaining fully stretched in the PNRSS pore at +20 mV. Norepinephrine report negative proceeding binding events at +160 mV. In summary, a fPNRSS strand may be permanently conjugated to the pore to further boost the resolution and consistency of PNRSS.

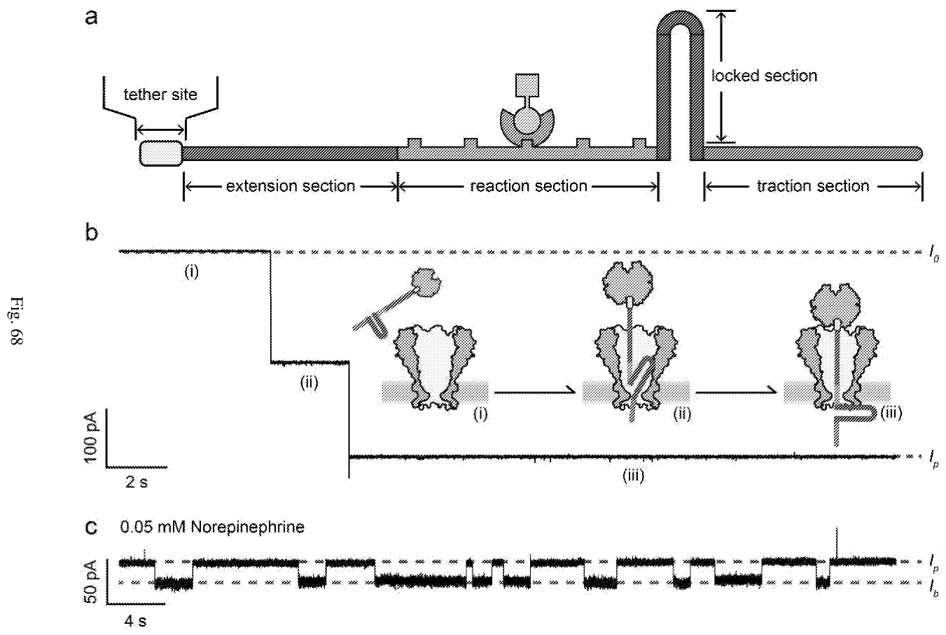

As shown in FIG. 68, we demonstrate another PNRSS method named locked-PNRSS (1PNRSS). A 1PNRSS strand contains a locked section, capable of forming hairpin structure through hydrogen bond interaction to avoid 1PNRSS strand escape from the pore (FIGS. 68a and b). In addition, 1PNRSS strand 14PBA contains a sole PBA, capable of binding norepinephrine. In this case, norepinephrine report negative proceeding binding events at +160 mV (FIG. 68c). In summary, on the basis of PNRSS method, we set a locked section on the PNRSS strand to avoid the stochastic escape of the PNRSS strand from the nanopore by forming a hairpin structure. 1PNRSS technology can prolong the measurement time and consistency.

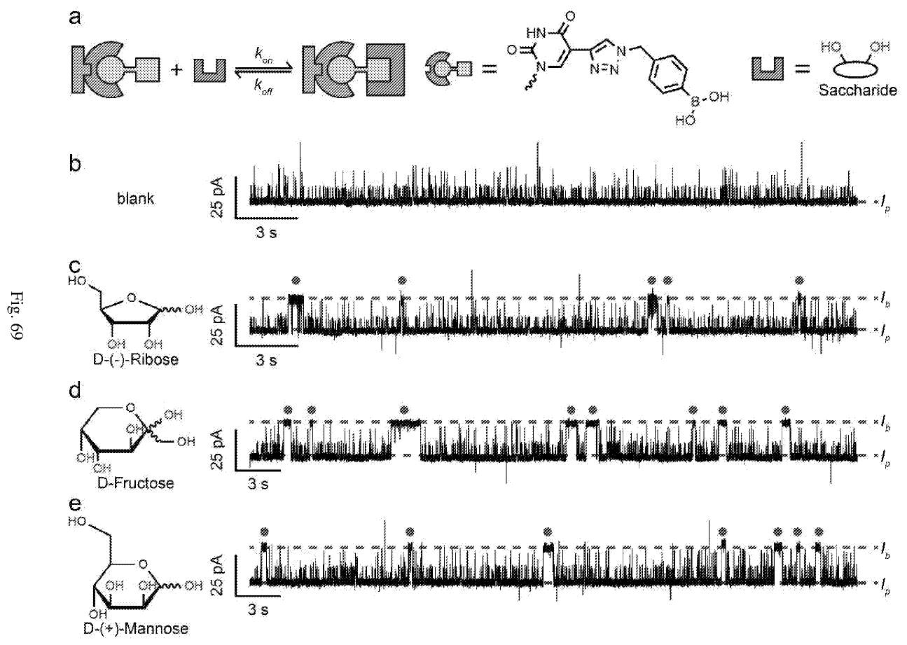

As shown in FIG. 69, we demonstrate the ability of PNRSS technology to sense saccharides. Three monosaccharides are shown here. And it's important to note that there are some signals in the background caused by Tris buffer. Tris buffer was used at the time could react with phenylboric acid to introduce the background. However, the events of Tris binding to PBA were apparently different from the events of saccharide binding to PBA. In summary, PNRSS technology, mainly introduced by phenylboric acid, can directly sense a variety of saccharides.

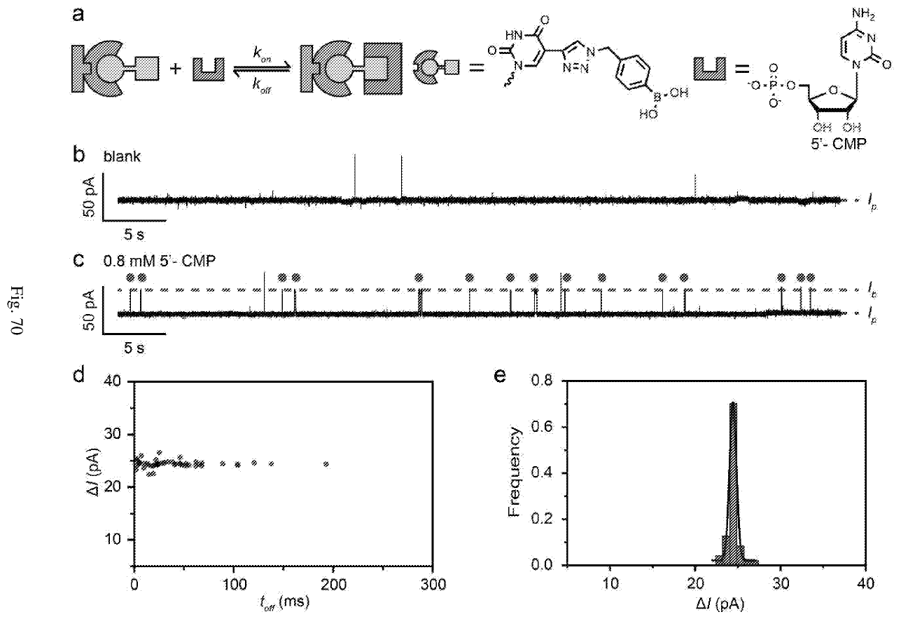

As shown in FIG. 70, we demonstrate the ability of PNRSS technology to sense nucleotides. It's important to note that we take 5′-monophosphate (5′-CMP) as an example here. However, nucleosides, nucleotides and nucleoside analogues having a diol structure can be observed directly by PNRSS technology in theory. In summary, PNRSS technology, mainly introduced by phenylboric acid, can directly sense a variety of nucleosides, nucleotides and nucleoside analogues.

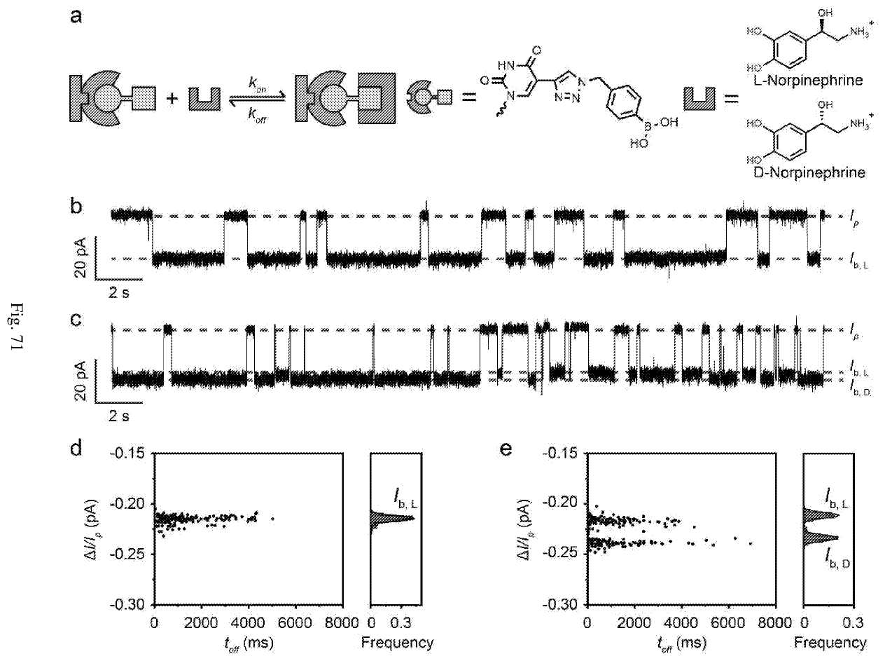

As shown in FIG. 71, we demonstrate the discrimination of PNRSS technology for molecular chirality. Here, we take enantiomeric norepinephrine as an example here and show signals of continuous addition of L and D-norepinephrine. In summary, PNRSS technology, mainly introduced by phenylboric acid, can directly distinguish molecular chirality of catecholamines.

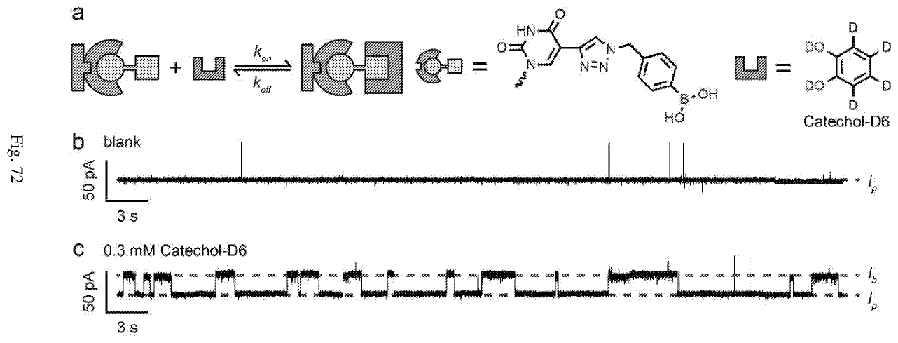

As shown in FIG. 72, we demonstrate the ability of PNRSS technology to sense a catechol with isotope in it, which the hydrogen on the molecule has been replaced by an isotope of deuterium. In summary, PNRSS technology, can directly sense a variety of molecule containing an isotope.

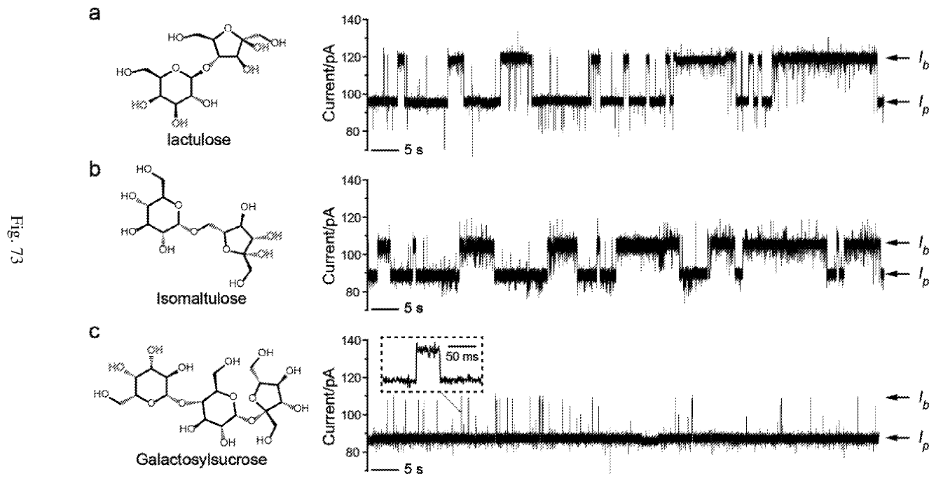

As shown in FIG. 73, we demonstrate the ability of PNRSS technology to sense polysaccharides. Three polysaccharides are shown here, including disaccharide and trisaccharide. All report clear and distinct binding events. In summary, PNRSS technology, mainly introduced by phenylboric acid, can directly sense a variety of polysaccharides.

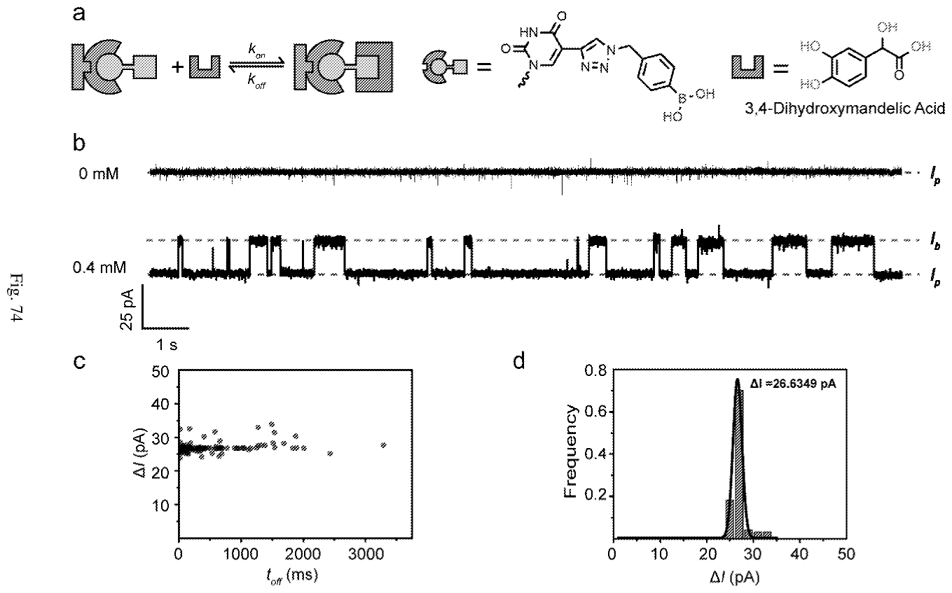

As shown in FIG. 74, we demonstrate the ability of PNRSS technology to sense a molecule containing two reaction groups. 3,4-dihydroxymandelic acid has two diols structure which can be reacted with phenylboric acid in two ways.

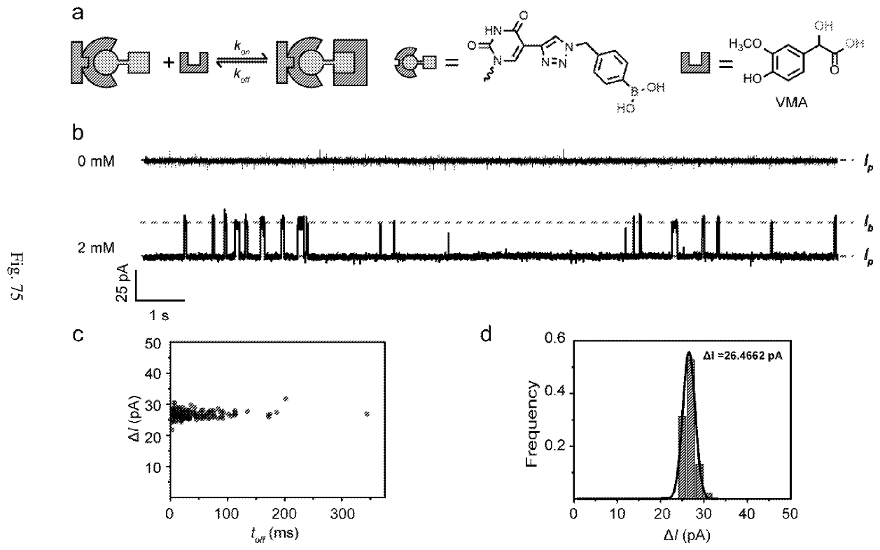

As shown in FIG. 75, we demonstrate the ability of PNRSS technology to sense 4-Hydroxy-3-methoxymandelic acid (VMA). VMA has a diol structure which can be reacted with phenylboric acid.

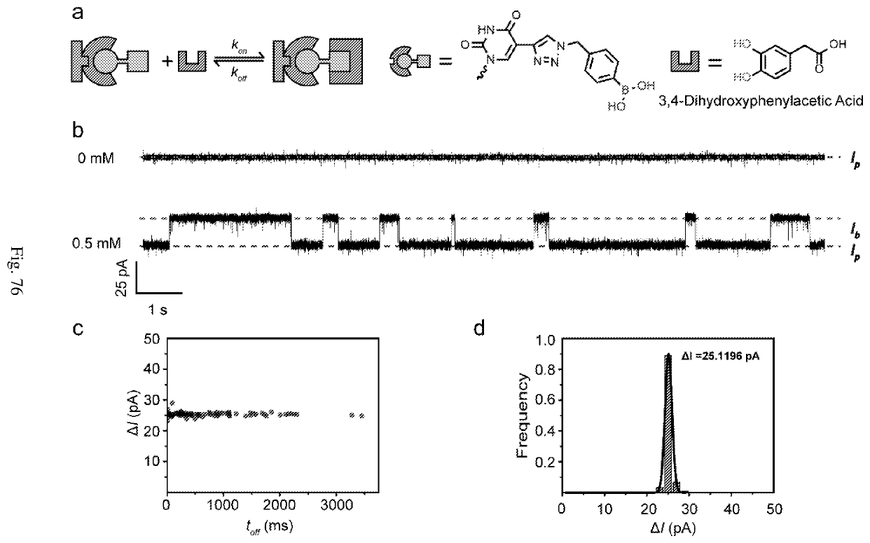

As shown in FIG. 76, we demonstrate the ability of PNRSS technology to sense 3,4-Dihydroxyphenylacetic acid. 3,4-Dihydroxyphenylacetic acid has a diol structure which can be reacted with phenylboric acid.

### Discussions

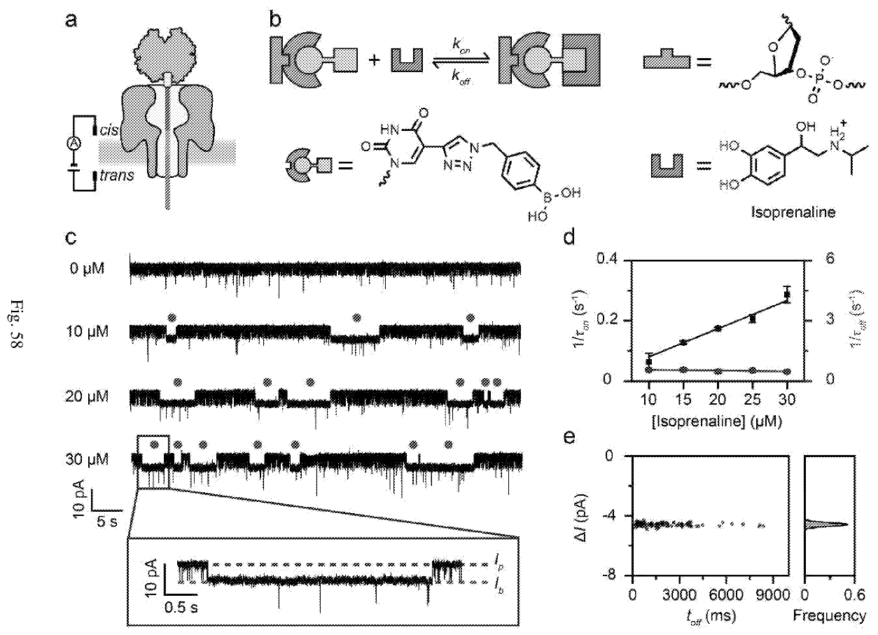

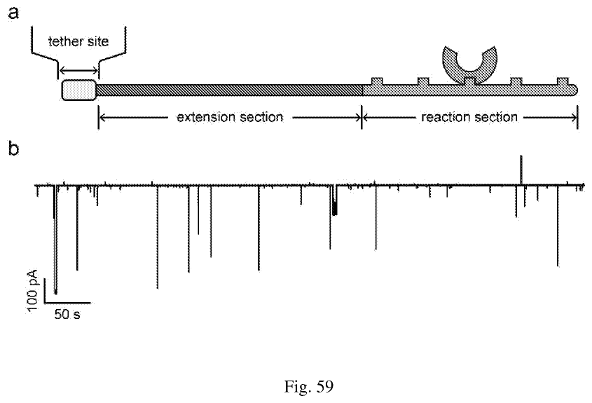

20 analytes have so far been analysed using MspA as the PNRSS pore. In principle, any nanopore in which a synthetic polymer could be tethered and fully stretched would be suitable to perform PNRSS. However, the performance is to be determined by the overall structural of this pore. To approve the generality of PNRSS to work with other channel proteins, a feasibility test was performed using wildtype α-hemolysin (WT α-HL) as the PNRSS pore and 14PBA (Table 1) as the PNRSS strand. Isoprenaline was applied as the mobile reactant (FIG. 58). Experimentally, events of isoprenaline binding, which appear as negative proceeding (I_{b}<Iₚ) resistive pulses, were successfully observed, confirming our hypothesis that PNRSS has a generality when used with other channel proteins. However, the reported event amplitude (˜−4.6 pA) is much smaller than that produced by MspA (˜−32.2 pA, Table 11), though other measurement conditions were kept identical. This experimentally suggests that MspA, which has an overall conical geometry, results in a more focused electrical field at the pore constriction thus a larger event amplitude was produced. It is thus an optimum choice of PNRSS pore to distinguish between analytes with similar chemical structures. A PNRSS strand with no traction section (14TAK-NTS, Table 1) was as well tested, which fails to report any successful trapping of the PNRSS strand (FIG. 59). Although the traction section is useful to maintain an electrophoretic force during the measurement, it should be understood that other methods can be used to hold the PNRSS strand, or an instantaneous measurement can be made.

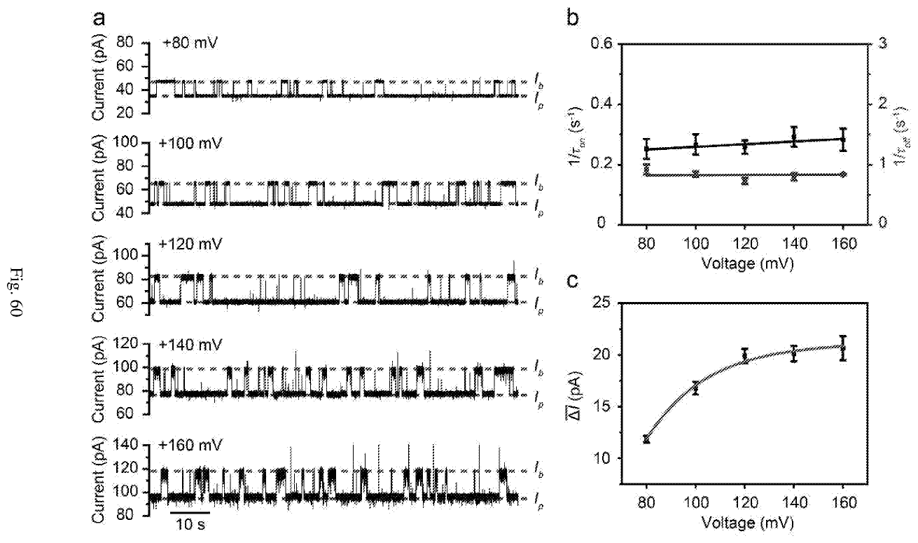

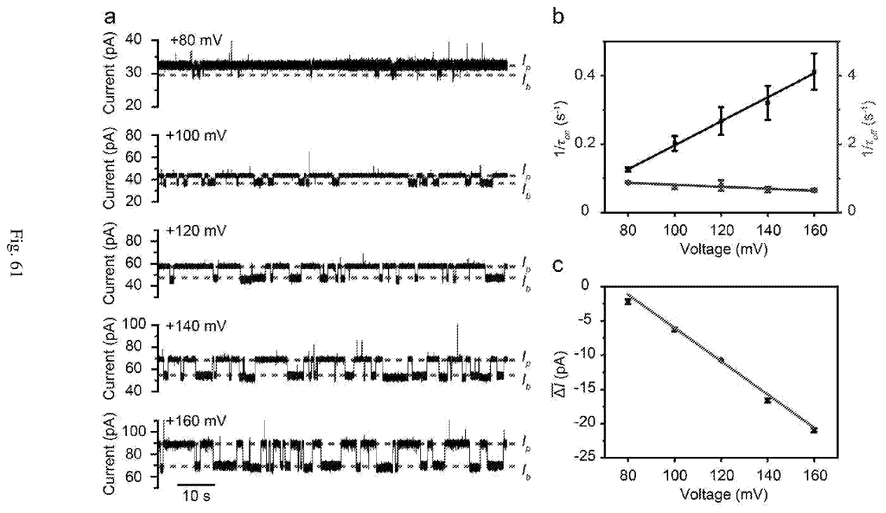

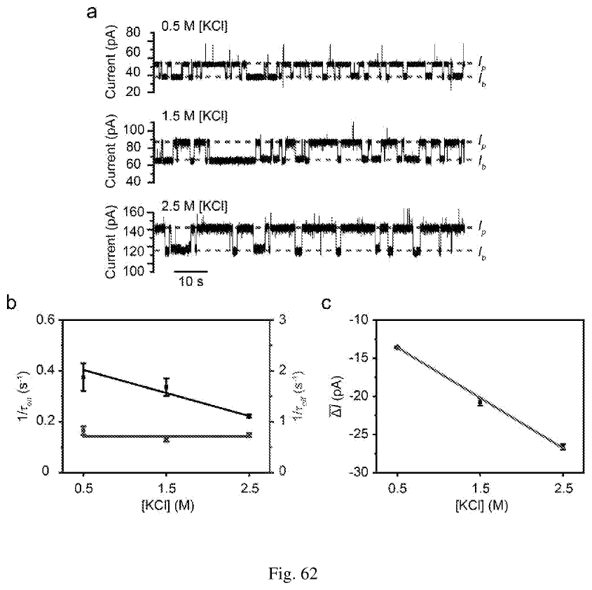

To be consistent all through the paper, the measurement condition was generally kept identical with which a buffer of 1.5 M KCl, 10 mM HEPES was used and a high voltage such as a +180 mV or a +160 mV was applied. By respectively taking catechol (FIG. 60) or norepinephrine (FIG. 61) as representative electrical neutral or electrical positive analyte, PNRSS measurements were carried out with a gradient of voltages between +80 mV and +160 mV. For both analytes, the reported event amplitude is generally larger when a higher potential was applied, suggesting that a higher applied potential is more advantageous by producing a higher sensing resolution. Though a +20 mV potential is enough to maintain the PNRSS strand in the pore lumen and backflow of the PNRSS strand is rarely observed, a minimum of +60 mV potential was required to produce a large enough event amplitude to be detectable. The event dwell time is generally independent of the amplitude of the applied potential for both analytes. However, norepinephrine which is positively charged, reports a clearly higher rate of event appearance when a larger potential was applied, indicating that the electrophoretic force is critical when the mobile reactant is charged (FIG. 61). In contrast, the rate of event appearance is less modulated by the applied potential for catechol, approving that the contribution of the electroosmotic flow is negligible (FIG. 60). PNRSS may also be carried out at different salt concentrations and generally a higher salt concentration is also more advantageous to produce a large event amplitude by producing a larger flow of ions through the pore (FIG. 62).

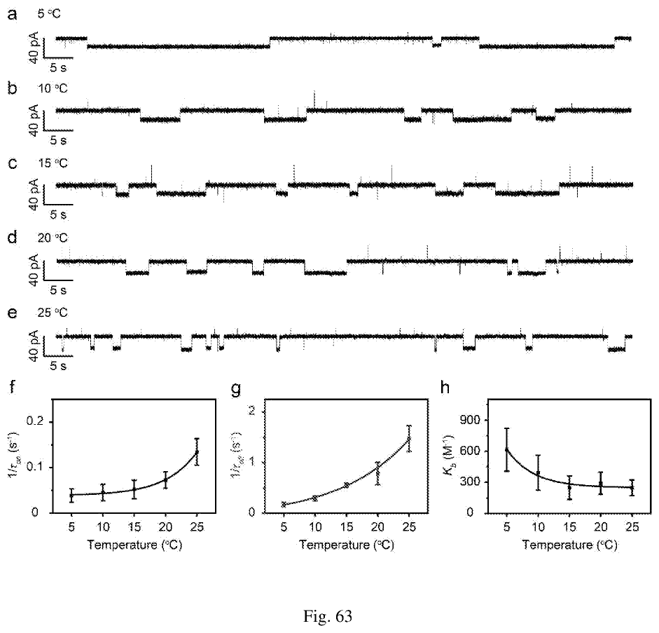

The rate of single molecule chemical reactions may as well be modulated by temperature. Experimentally, by taking norepinephrine as a model analyte and phenylboronic acid as the fixed reactant, PNRSS was carried out on an Orbit Mini nanopore reader (Nanion Technologies GmbH, Germany) with a built-in temperature control module (FIG. 63). Clearly, both the on and the off rate and the binding affinity is modulated by the temperature. The reaction rates are generally exponentially related with the set temperature which fit the rule as described by an Arrhenius equation⁷⁸. The trans compartment of the Orbit Mini chip is too small to place the mobile analyte, thus in this measurement, norepinephrine was added to cis, different from that performed in FIG. 43. However, events were detectable in both configurations, confirming that fast diffusion of the small molecule analyte is also contributing to the generation of events.

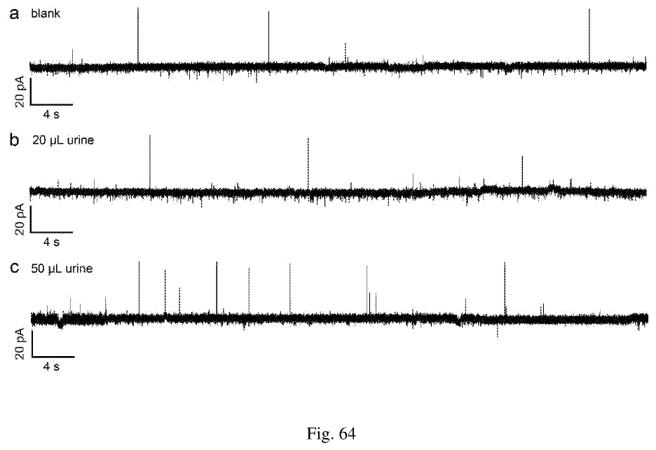

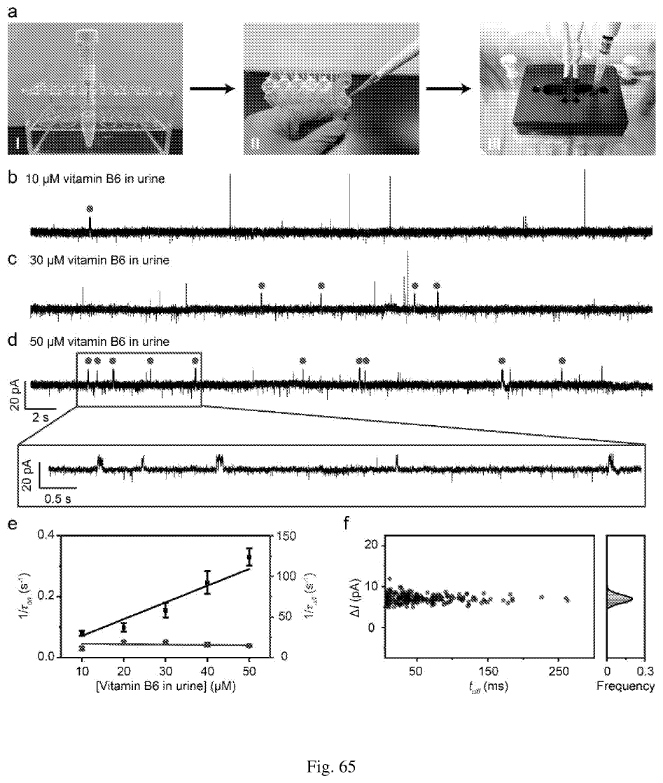

In this paper, the limit of detection is specifically defined as the minimum final analyte concentration in the measurement chamber to acquire at least 5 events during a 10 min continuous measurement (Table 14). Due to the large volume of the measurement chamber and the small size of a single nanopore sensor, the efficiency of detection is generally not optimum for the current configuration. Thus, without any sample enrichment, it is not expected to observe enough events for quantification when a target analyte was present in a low concentration in a physiological sample. This is the case for epinephrine, norepinephrine and isoprenaline which have a ˜ nM physiological concentration in blood serum⁷⁹ however the detection limit reported here is ˜1 μM. Engineering wise, this may be improved by introducing thousands of independent parallel nanopore sensors and an extremely flat flow cell to boost the sensing efficiency, similar to a configuration demonstrated in a MinION sequencer¹⁹. On the other side, some analyte tested in this paper may appear at a high concentration in natural samples and may be directly applied for detection even with the current setup. Vitamin B6 reports an easily recognized event pattern during PNRSS and the detection limit is ˜400 nM. We thus designed an assay to mimic detection of Vitamin B6 in true human urine samples. Experimentally, addition of true human urine sample to trans didn't report any interfering events (FIG. 64). However, test of human urine samples with added vitamin B6 reported corresponding events, suitable for direct quantification according to the calibration curve reported (FIG. 65). These results indicate that the PNRSS assay is ready to perform tests with true biological samples, similar to other state-of-the-art nanopore assays^{80,81}. A unique event pattern is helpful to assist event recognition from a heterogeneous solution.

### Limitations

However, the above demonstration is still away from perfection. For quick demonstrations, all PNRSS were performed with the pore and the strand chemically separated. Though this sensing mode is advantageous for repetitive measurement of irreversible reactions, a PNRSS strand may be permanently conjugated to the pore to further boost the resolution and consistency of PNRSS. The triazole produced by the CuAAC reaction may for example report undesired chemical reactions with transient metal ions (FIG. 2). This interference may however be minimized by the addition of chelating agent such as ethylenediaminetetraacetic acid (EDTA) or by the choice of an alternative conjugation chemistry 82. Results in this manuscript might be inspiring in preparation of chemical compounds. However, due to the small scale of this single molecule reactor, PNRSS is designed as a sensing instead of a preparative method at the moment. The K_{b} values measured by PNRSS were also compared with those reported in literatures (Table 15). It is however difficult to find results describing all chemical reactions reported in this paper and performed at an exactly identical condition but a general consistency of result is seen. However, we would like to emphasize that the purpose to develop PNRSS is to apply existing knowledges of chemical interactions between reactants to achieve direct chemical sensing of small molecular analytes, which has been well supported by results in this paper.

### Conclusions

To summarize, PNRSS, first reported in this paper, serves as a convenient molecular toolkit with which to study single molecule chemistry processes with a nanopore. To better illustrate the idea of PNRSS, an artistic video demonstration (Video 7) was included. The specific aim is to break the technical bottleneck to introduction of any number or type of reactive groups into any spot of the nanopore lumen, which is difficult, even for the best in the field. With PNRSS, however, this difficulty has been transformed instead to synthesis of functional DNA oligomers, a routine performed daily by countless biochemistry labs or as a low-cost service provided by a variety of commercial vendors. Distinct from the previous configuration, PNRSS has also enabled repetitive monitoring of irreversible reactions, further broadening the choice of chemical reactions that previously were difficult to study by a nanopore. We report a sum of 20 single molecule chemical reactions, in which hydrogen peroxide, buffer reagent, transition metal ions, glycerol, lactic acid, vitamins, catecholamine derivatives or anti-viral medicines participate. The reported event patterns are highly diversified, associated with the size, charge and conformation of the analytes, useful for single molecule recognition. Though limited by the length of this paper, these reactions demonstrate core aspects of the PNRSS technique, and show its feasibility and versatility. To the best of our knowledge, this is the largest number of nanopore based single molecule chemistry reactions that have been reported in a single publication. Most of them have never been previously investigated as single molecules (Table 16). Given the conical structure of MspA, which efficiently focuses ionic flows to its narrow pore restriction, all single molecule chemical reactions discussed in this paper have demonstrated a fully resolved event amplitude. For PBA, direct recognition of epinephrine, norepinephrine and isoprenaline also suggests its immediate biomedical applications. The single molecule discrimination of remdesivir and its triphosphate metabolite by PNRSS may inspire pharmacokinetics measurements with a variety of nucleoside analogue medicines.

In subsequent studies, PNRSS may be carried out with multiple fixed reactants on the same strand, thus increasing the complexity of sensing. Though demonstrated with MspA and α-HL⁶, other biological nanopores such as aerolysine 8 or CsgG⁹ are in principle also compatible with PNRSS, as long as a tethered polymer containing the designed reactive site can be fully stretched in the pore lumen. Nanopores such as cytolysin A (ClyA)¹⁰, fragaceatoxin C (FraC)¹¹, pleurotolysin (PlyA/B)¹² or phi29 Connector¹⁴ with large openings may as well be applied to probe chemical reactions involving larger or more complex mobile reactants, such as polysaccharides or cyclopeptides. However, these proposed plans to apply PNRSS with large channel proteins have not been carried out yet but may be inspiring to other colleagues in the field.

### Author Contributions

S. H. and W. D. J. conceived the project. W. D. J. and C. Z. H. performed the measurements. Y. M. G. and J. M. performed the molecular simulations. G. R. Q. designed the machine-learning algorithms. Y. Q. W., Y. L. S. H. Y. J. C. and S. Y. Z. prepared the MspA nanopores. W. D. J. and X. Y. D performed the measurements with α-HL. W. D. J. and L. Y. W performed the measurements at different temperatures. P. K. Z. set up the instruments. S. H. and W. D. J. wrote the paper. Y. Q. W. and W. D. J. prepared the supplementary videos. S. H. and H. Y. C. supervised the project.

### Data and Code Availability Statement

All data and code presented in this work can be requested from the corresponding author upon reasonable request.

### Competing Interest Statement

S. H. and W. D. J. have filed patents describing the PNRSS technology and its applications thereof. G. R. Q. is the founder of Intelligence Qubic Technology Co. Ltd, a company engaged in the development of artificial intelligence software interfaces. The authors claim no other competing interest.

### Acknowledgments

The authors acknowledge Prof. Hagan Bayley (University of Oxford) for valuable suggestions during preparation of the manuscript. The authors acknowledge Prof. Zijian Guo, Prof. Shaolin Zhu, Prof. Congqing Zhu, Prof. Jie Li and Prof. Ran Xie in Nanjing University, Mrs. Yiou Ma, Prof. Daoqiang Zhang, Xiaoyu Guan in Nanjing University of Aeronautics and Astronautics for inspiring discussions. S. H. is grateful for encouragement from his grandmother, Mrs. Jiqing Xia during the completion of this project. Unfortunately, due to aging, Mrs. Xia passed away prior to the manuscript submission.

This project was funded by National Natural Science Foundation of China (No. 31972917, No. 91753108, No. 21675083), Fundamental Research Funds for the Central Universities (No. 020514380257, No. 020514380261), State Key Laboratory of Analytical Chemistry for Life Sciences (No. 5431ZZXM1902, No. 5431ZZXM1804), Natural Science Foundation of Jiangsu Province, Programs for high-level entrepreneurial and innovative talents introduction of Jiangsu Province (individual and group program). Technology innovation fund program of Nanjing University. Excellent Research Program of Nanjing University (Grant No. ZYJH004).

### Materials

Pentane, hexadecane, ethylenediaminetetraacetic acid (EDTA), Genapol X-80 were obtained from Sigma-Aldrich. 1,2-diphytanoyl-sn-glycero-3-phosphocholine (DPhPC) was supplied by Avanti Polar Lipids. Potassium chloride (KCl, 99.9%), sodium hydroxide (NaOH, 99.9%), cobalt sulfate heptahydrate (CoSO₄·7H₂O, 99.99%), nickel sulfate hexahydrate (NiSO₄·6H₂O, 99.9%), copper sulfate pentahydrate (CuSO₄·5H₂O, 99.9%), zinc sulfate heptahydrate (ZnSO₄·7H₂O, 99.995%), cadmium sulfate, 8/3-hydrate (CdSO₄·8/3H₂O, 99.99%), ethylene glycol (99.9%), glycerol (99.7%), L-lactic Acid (98%), pyridoxine (vitamin B6) (98%), 30% hydrogen peroxide solution (H₂O₂, GR), DL-norepinephrine hydrochloride (97%), DL-epinephrine hydrochloride (98%), DL-isoproterenol hydrochloride (99%), 3-azidopropylamine (95%), sodium sulfate anhydrous (Na₂SO₄, 99%), dimethyl sulfoxide (DMSO, 99.9%) and dimethyl sulfoxide-d6 (DMSO-d6, D.99.9% +0.03% TMS) were from Aladdin (China). Methylboronic acid (97%), catechol (99.5%), resorcinol (AR) and acetonitrile (MeCN, 99.9%) were from Macklin (China). Hydrochloric acid (HCl), acetone (Me₂CO, 99.5%) and dichloromethane (DCM, 99.5%) were from Sinopharm (China). Sodium ascorbate (vitamin C) (99%), 4-(2-hydroxyethyl)-1-piperazine ethanesulfonic acid (HEPES, 99%) were purchased from Shanghai Yuanye Bio-Technology (China). 4-(Azidomethyl)benzeneboronic acid pinacol ester (95%) was from Alfa Aesar (U.S.). Remdesivir (99.74%) and remdesivir metabolite (99.87%) were purchased from MedChemExpress (Monmouth Junction, NJ, USA).

E. coli strain BL21 (DE3) was from Biomed (China). Streptavidin was from New England Biolabs. Dioxane-free isopropyl-β-D-thiogalactopyranoside (IPTG), kanamycin sulfate and tris-(Hydroxy-methyl) aminomethane (Tris) were from Solarbio Biotechnology (China). Luria-Bertani broth and Luria-Bertani agar were from Hopebio (China). Precision Plus Protein™ Dual Color Standards and TGX™ FastCast™ Acrylamide Kit (12%) were purchased from Bio-Rad.

The monomeric DNA phosphoramidite, 5-ethynyl-dU-CE phosphoramidite was purchased from Glen Research (U.S.), and the alkyne-containing oligonucleotide 14TAK (Table 1) was synthesized by Shanghai Generay Biotech Co., Ltd. All other DNA oligonucleotides were synthesized by Genscript (New Jersey, U.S.). Full sequences are listed in Table 1.

### Methods

### 1. Nanopore Preparation

The gene coding for the monomeric M2 MspA mutant (D93N/D91N/D90N/D118R/D134R/E139K) was synthesized and inserted in a pet-30a(+) vector. The M2 MspA was expressed with E. coli BL21 (DE3) and purified using nickel affinity chromatography (GE Akta Pure, GE Healthcare), as previously reported¹. The purified M2 MspA spontaneously oligomerizes into an axis symmetric, octameric form, ready for all PNRSS measurements in this paper. The octameric M2 MspA is the sole nanopore used in this work. For simplicity, it is referred to as MspA throughout the paper, unless otherwise stated.

The gene coding for the monomeric wildtype α-hemolysine (WT α-HL) was synthesized and inserted in a pet-30a(+) vector. Preparation of heptameric WT α-HL was carried out strictly following that previously reported⁸³.

The plasmid DNAs coding for M2 MspA and WT α-HL have been shared with access code MC_0101191 and MC_0068416 in the molecular cloud plasmid repository (https://www.molecularcloud.org/s/shuo-huang, GenScript, New Jersey). Citation is requested when publishing with this plasmid.

### 2. Nanopore Measurements and Data Analysis

Nanopore measurements were carried out in a custom measurement chamber. A self-assembled lipid bilayer is formed by 1,2-diphytanoyl-sn-glycero-3-phosphocholine (DPhPC), separating the chamber into the cis and the trans compartments. Each compartment is filled with 500 μL electrolyte buffer of 1.5 M KCl, 10 mM HEPES, pH 7.0 or 8.0. All measurements with the PNRSS strands 14X, 13G/14G, 14A, 14G and 14TAZ (Table 1) were conducted at pH 7.0. When tris was not used as the mobile reactant, all measurements with the PNRSS strand 14PBA (Table 1) were accomplished at pH 8.0. For all measurements with tris, the pH was adjusted to either 7.0 or 8.0. A pair of Ag/AgCl electrodes was respectively placed in cis and trans sides of the chamber, in contact with the aqueous buffer on each side. The Ag/AgCl electrodes were electrically connected to a patch clamp amplifier to form a closed circuit. By convention, the electrode in the cis compartment is electrically grounded while the opposing electrode is the working electrode.

To obtain the mean blockage level (I̅ₚ̅), a static pore blockage measurement was performed, using a published method⁸⁴. Briefly, with a single MspA inserted, the PNRSS strand was added to cis with a 20 nM final concentration. A voltage protocol of +180 mV or +160 mV (0.9 s) and −100 mV (0.3 s) was applied repeatedly and Iₚ was measured when the +180 mV or the +160 mV potential was applied. A minimum of 500 Iₚ events were collected during each experiment. The events were fit to a Gaussian distribution. I̅ₚ̅ was derived from the central position of the fitting. Three independent measurements were performed to obtain the mean and the standard deviation of I̅ₚ̅.

To perform PNRSS, the desired PNRSS strand was added to cis with a 10 nM final concentration. A positive potential was continuously applied. The PNRSS events were recognized as further pore blockage events, on top of the Iₚ level (FIG. 10).

All electrophysiology recordings were carried out with an Axopatch 200B patch clamp amplifier. The acquired traces were digitized by a Digidata 1550B analog-to-digital converter (Molecular Devices, UK) with a 25 kHz sampling rate and low-pass filtered with a corner frequency of 1 kHz. Experimentally, MspAs were added to cis for spontaneous single pore insertion. With a single pore inserted in the membrane, the electrolyte buffer in cis was exchanged to avoid further pore insertions. All PNRSS measurements were conducted at room temperature (21±2° C.). Nanopore events were extracted by the single channel search feature of Clampfit 10.7 (Molecular Devices, UK). Further analysis was carried out in Origin 2019. All color coded scatter plots were generated by ggplot2, an R package used for data visualization.

### 3. Streptavidin DNA Conjugation

To form streptavidin-tethered DNA complexes, DNA oligomers with a 5′ biotin-TEG modification (Table 1) were incubated with streptavidin with an equal molar ratio at room temperature (rt) for 10 min. During a PNRSS measurement, the formed streptavidin-tethered DNA complex was added to cis at the desired final concentration.

### 4. Theoretical Calculations of Optimized Binding Configurations

All theoretical calculations were performed with the Gaussian 16 package suite⁸⁵. Geometry optimizations were carried out using density functional theory (DFT) with the M06 functional^{86,87}. The 6-31+G(d) basis set was employed for C, H, O, N, and P atoms, while the LANL2DZ basis set, together with the related effective core potentials⁸⁸, was used for Ni atoms. The relative energy (ΔE) between the systems of low-spin (E_{LS}) and high-spin (E_{HS}) state was computed from:

ΔE=E_{LS}−E_{HS} (1)

Possible binding modes of Ni²⁺ with low-spin (LS) and high-spin (HS) states were investigated, as shown in FIG. 9. Two modes of Ni²⁺ cluster with 4 or 5H₂O molecules were calculated for the following study. The computational results of relative energy, ΔE, between different spin states, are listed in Table 3. The HS states of (dGMP)₂-Ni-4 wt and (dGMP)₂-Ni-5 wt were −44.13 and −36.85 kcal/mol lower than those of LS states, respectively, indicating that the HS states are energetically favorable. The geometry of HS states displayed a 6-coordination octahedral structure with the distance between Ni and N (O) atoms of 2.0-2.1 Å. However, the octahedral geometry showed the distortion to some extent in LS states. The O—H . . . O and O—H . . . N hydrogen bonding interaction plays an important role in the binding with the Ni²⁺ cluster.

The binding energy (E_{b}) was calculated to investigate the binding ability of different modes. The binding energy was obtained by calculating the energy difference between the total energy of the complex system (E) and the sum of individual energy of the H₂O (E_{wt}), the Ni²⁺ ion (E_{Ni}) and two deoxyguanosine monophosphates (E_{dGMP/dGMP}), respectively, and was calculated from:

E_{b}=E−xE_{wt}−E_{Ni}−E_{dGMP/dGMP} (2)

where x is the number of H₂O molecules. A Ni²⁺ ion can bind with four H₂O and two guanine molecules, showing the binding energy with −45.33 kcal/mol. For (dGMP)₂-Ni-5 wt, a Ni²⁺ ion can bind one guanine and five H₂O molecules with the E_{b} of −46.07 kcal/mol, in which one H₂O molecule can bind the guanine via the N(7) atom by hydrogen bonding interaction.

### 5. The Introduction of Functional Azides to a PNRSS Strand

An alkyne-containing DNA strand 14TAK (Table 1) was applied as a universal PNRSS strand template to introduce any functional azides. Functional azides, such as 3-azidopropylamine (FIG. 17-18) or 4-(azidomethyl) benzeneboronic acid (FIG. 24-26), were chemically conjugated by a Huisgen copper (I)-catalyzed azide-alkyne 1,3-dipolarcycloaddition (CuAAC) reaction.

### 6. The Procedure to Produce 4-(Azidomethyl) Benzeneboronic Acid

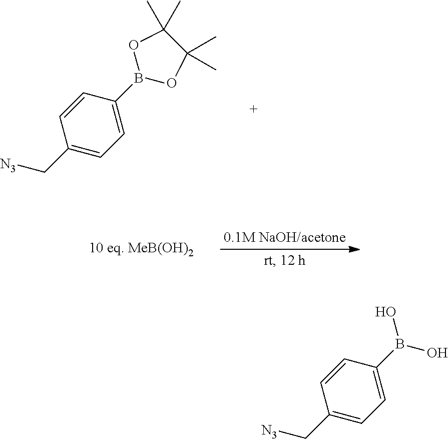 [SMILES: CC1(C)OB(c2ccc(CN=[N+]=[N-])cc2)OC1(C)C ; [N-]=[N+]=NCc1ccc(B(O)O)cc1]

4-(azidomethyl) benzeneboronic acid pinacol ester (158 mg, 0.6 mmol) and methylboronic acid (360 mg, 6 mmol) were added to a 10 mL reaction tube, and dissolved in acetone (2 mL). After further addition of 0.1 M NaOH (2 mL), the resulting solution was stirred at rt for 12 h. Afterwards, 4 mL dichloromethane was added to the solution. The resulting mixture was poured into a separatory funnel to remove the organic layer. The pH of the solution was adjusted to 7 by titration with 0.1 M HCl. And then, 4 mL dichloromethane was added in water layer to extract the target product. The organic layer was washed with H₂O to remove residual salts. The organic phase was further dried with solid Na₂SO₄. The organic solvent was removed with a rotary evaporator to collect the 4-(azidomethyl) benzeneboronic acid as a white powder (74.2 mg, 68% yield)⁸⁹. The product was further characterized by ¹H NMR spectroscopy to confirm the success⁹⁰ (FIG. S18).

TABLE 1
Sequence context of all PNRSS.
strands in this study

| PNRSS | |
|---|---|
| Strand | Sequence |
| 14TAK-NS | 5′-biotin TEG-TTTTTTTTTTXX(TAK)XX-3′ |
| 13G/14G | 5′-biotin TEG-TTTTTTTTTTXXGGXXTTTTTTTTT |
| | TTTTTTTTTTTTTTTTTTTTTTTTTTTTTTTTTTT-3′ |
| 14A | 5′-biotin TEG-TTTTTTTTTTXXAXXTTTTTTTTTT |
| | TTTTTTTTTTTTTTTTTTTTTTTTTTTTTTTTTTT-3′ |
| 14G | 5′-biotin TEG-TTTTTTTTTTXXGXXTTTTTTTTTT |
| | TTTTTTTTTTTTTTTTTTTTTTTTTTTTTTTTTTT-3′ |
| 14X | 5′-biotin TEG-TTTTTTTTTTXXXXXTTTTTTTTTT |
| | TTTTTTTTTTTTTTTTTTTTTTTTTTTTTTTTTTT-3′ |
| 14TAK | 5′-biotin TEG-TTTTTTTTTTXX(TAK)XXTTTTTT |
| | TTTTTTTTTTTTTTTTTTTTTTTTTTTTTTTTTTTTTT |
| | T-3′ |
| 14TAZ | 5′-biotin TEG-TTTTTTTTTTXX(TAZ)XXTTTTTT |
| | TTTTTTTTTTTTTTTTTTTTTTTTTTTTTTTTTTTTTT |
| | T-3′ |
| 14PBA | 5′-biotin TEG-TTTTTTTTTTXX(PBA)XXTTTTTT |
| | TTTTTTTTTTTTTTTTTTTTTTTTTTTTTTTTTTTTTT |
| | T-3′ |

### Footnotes:

1. The reaction sections are marked with bold fonts in each sequence.
2. Natural DNA bases or their combinations such as A, G or GG can serve as fixed reactants.
3. X represents an abasic site, which is incapable of binding any mobile reactant tested in this study.
4. The 5′ Biotin TEG serves as the tether spot, forming tight binding with a streptavidin stopper.

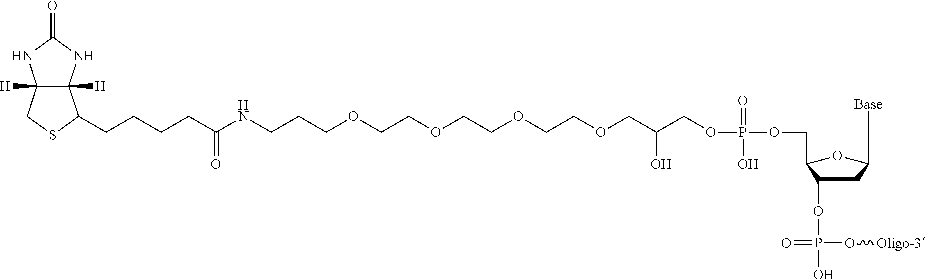

5. (TAK) is an alkyne-modified thymine analogue (5-ethynyl deoxyUridine, Glen Research, U.S.), serving as a universal connector to introduce functional azides. The chemical structure of (TAK) is provided below:

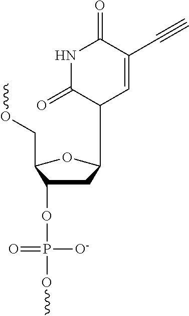

6. TAZ stands for 1,2,3-triazole. The chemical structure of the nucleotide containing a TAZ is provided below. Detailed procedures of its chemical synthesis and characterizations are provided in Methods and FIG. 17-18.

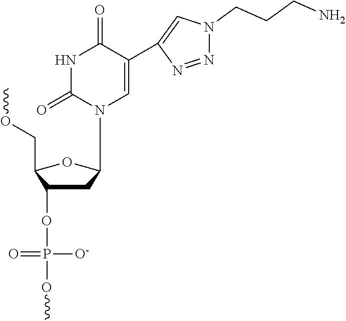

7. PBA) stands for phenylboronic acid. The chemical structure of the nucleotide containing a TBA is provided below. Detailed procedures of its chemical synthesis and characterizations are provided in Methods and FIG. 25-26.

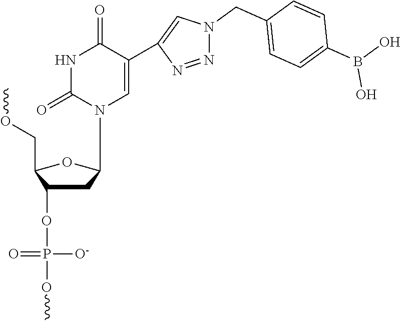

TABLE 2
Statistics of Īₚ of 14X, 14A, 14G or 13/14G blockages. The static
pore blockage measurements were performed as described in
Methods. A buffer of 1.5M KCl, 10 mM HEPES, pH 7.0
was applied. Each PNRSS strand was added to cis with a final
concentration of 20 nM. Iₚ was measured when a +180 mV bias
was applied (FIG. 10). 500 events were acquired during each
measurement. Three independent measurements (N = 3) were
performed for each condition to produce the statistics.

| PNRSS strand | Īₚ (pA) |
|---|---|
| 14X | 143.2 ± 1.2 |
| 14A | 144.5 ± 1.3 |
| 14G | 145.9 ± 0.4 |
| 13G/14G | 160.3 ± 0.3 |

TABLE 3
The relative energy of (dGMP)₂-Ni-4wt and (dGMP)₂-Ni-5wt.

| Systems | E_{LS} (a.u.) | E_{HS} (a.u.) | ΔE (kcal/mol) |
|---|---|---|---|
| (dGMP)₂-Ni-4wt | −2890.99584 | −2891.06616 | −44.13 |
| (dGMP)₂-Ni-5wt | −2967.38078 | −2967.43951 | −36.85 |

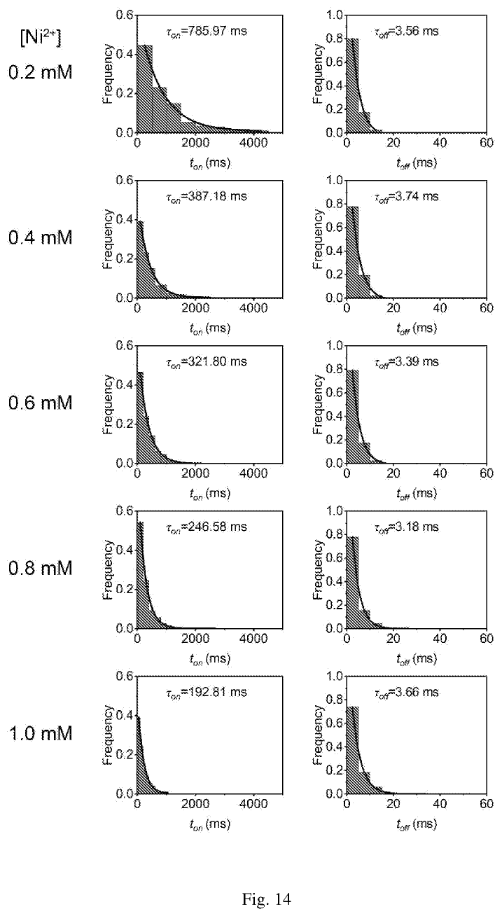

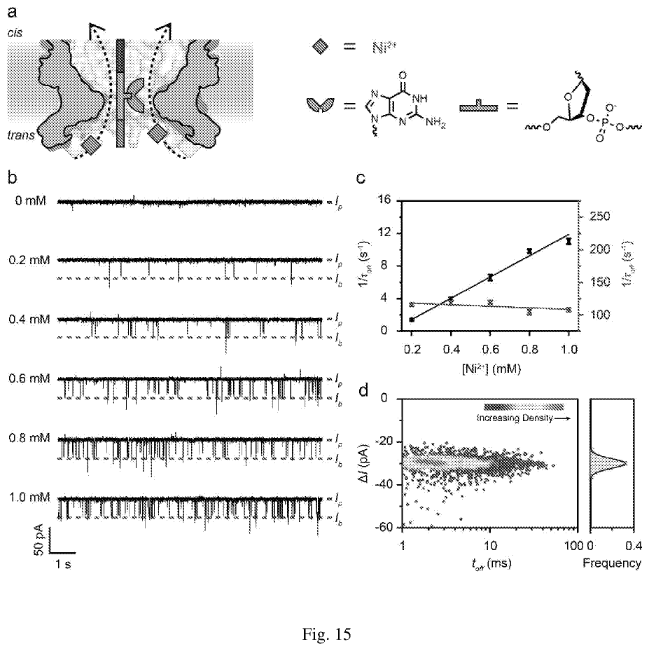

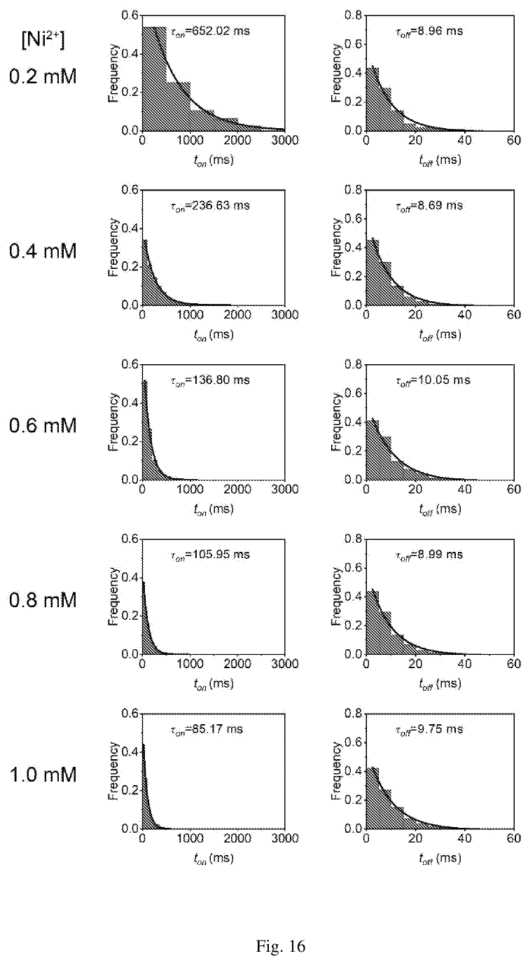

TABLE 4
Statistics of Δ̅I̅ and τ_{off} of Ni²⁺ binding to 14A, 14G or 13G/14G.
The PNRSS measurements were performed as described in FIG. 13
(14A), FIG. 15 (14G) and FIG. 1 (13G/14G). A buffer of 1.5M KCl,
10 mM HEPES, pH 7.0 was used. A +180 mV potential
was continuously applied. Ni²⁺ binding to 14A results in two event
populations (FIG. 13-14) so that the Δ̅I̅ and the τ_{off} values were derived
separately. Ni²⁺ binding to 14G (FIG. 15-16) or 13G/14G (FIG. 1)
respectively, reports a single event population. A minimum of 1000
events were included for each measurement.
Three independent measurements (N = 3) were
performed for each condition to generate the statistics.

| PNRSS strand | Δ̅I̅ (pA) | τ_{off} (ms) |
|---|---|---|
| 14A | −42.4 ± 1.7/−57 ± 2 | 5.68 ± 0.09 / 3.1 ± 0.4 |
| 14G | −28.1 ± 1.3 | 9.4 ± 0.3 |
| 13G/14G | −60 ± 3 | 11.0 ± 0.2 |

TABLE 5
Statistics of Īₚ for 14TAK and 14TAZ. The static pore blockage
measurements were performed as described in Methods. A buffer of
1.5M KCl, 10 mM HEPES, pH 7.0 was applied. Each PNRSS strand was
added to cis with a final concentration of 20 nM. In was measured
when a +180 mV bias was applied (FIG. 10). 500 events were acquired
from each measurement. Three independent measurements (N = 3)
were performed for each condition to generate the statistics.

| PNRSS strand | Īₚ (pA) |
|---|---|
| 14TAK | 137.8 ± 0.6 |
| 14TAZ | 92.5 ± 1.6 |

TABLE 6
Statistics of Δ̅I̅, τ_{off} and K_{b} of Ni²⁺ or Co²⁺ binding to a TAZ. The
PNRSS measurements were performed as described in FIG. 19 (Ni²⁺) and
FIG. 21 (Co2²⁺). A buffer of 1.5M KCl, 10 mM HEPES, pH 7.0 was used.
A +180 mV potential was continuously applied. A minimum of 1000
events were included for each measurement. Three independent
measurements (N = 3) were performed for each
condition to produce the statistics.

| Mobile Reactants | Δ̅I̅ (pA) | τ_{off} (ms) | K_{b} (M⁻¹) |
|---|---|---|---|
| Ni²⁺ | −19.1 ± 1.8 | 220 ± 50 | 390 ± 90 |
| Co²⁺ | −22 ± 3 | 1.32 ± 0.11 | 48 ± 14 |

TABLE 7
Statistics of Īₚ for PNRSS strand 14TAK and 14PBA. The static pore
blockage measurements were performed as described in Methods. A buffer
of 1.5M KCl, 10 mM HEPES, pH 8.0 was applied. Iₚ was measured when
a +160 mV bias was applied. 500 events were acquired from each
measurement. Three independent measurements (N = 3) were
performed for each condition to produce the statistics.

| PNRSS strand | Īₚ (pA) |
|---|---|
| 14TAK | 115.6 ± 0.4 |
| 14PBA | 99.8 ± 0.2 |

TABLE 8
Statistics of Δ̅I̅ and τ_{off} of phenylboronic acid binding with diols.
The PNRSS measurements were performed as described in FIG. 3.
A buffer of 1.5M KCl, 10 mM HEPES, pH 8.0 was used. Each type of
mobile reactant was separately added to trans with a final concentration
of 400 μM (catechol), 14 mM (ethylene glycol), 10 mM (glycerol), 4 mM
(L-lactic acid), 1.6 mM (vitamin C), 40 μM (vitamin B6).
A +160 mV bias was continuously applied during the measurement.
The definition and derivation of Δ̅I̅ and τ_{off} are described
in FIG. 10. For each measurement, Δ̅I̅ and τ_{off} values were derived
from events within a 15 min continuously recorded trace. Three
independent measurements (N = 3) were performed for
each condition to produce the statistics.

| Mobile Reactants | Δ̅I̅ (pA) | τ_{off} (ms) |
|---|---|---|
| Catechol | 24.9 ± 0.9 | 1030 ± 80 |
| Ethylene glycol | 23.4 ± 1.5 | 2.6 ± 0.2 |
| Glycerol | 22.2 ± 0.9 | 13.1 ± 0.3 |
| L-Lactic acid | 25.2 ± 1.2 | 15.6 ± 1.0 |
| Vitamin C | 26 ± 3 | 1.9 ± 0.2 |
| Vitamin B6 | 8.1 ± 0.5 | 41.9 ± 1.4 |

TABLE 9
Kinetic constants for the formation of complexes between PBA and diols.
The PNRSS measurements were performed as described in FIG. 27-38.
A buffer of 1.5M KCl, 10 mM HEPES, pH 8.0 was used. A +160 mV
potential was continuously applied. For each measurement, kinetic
constant values were derived from events within a 15 min continuously
recorded trace. Three independent measurements (N = 3) were
performed for each condition to produce the statistics.

| Mobile Reactants | kₒₙ (M⁻¹s⁻¹) | k_{off} (s⁻¹) | K_{b} (M⁻¹) |
|---|---|---|---|
| Catechol | 1000 ± 300 | 0.98 ± 0.08 | 1100 ± 300 |
| Ethylene glycol | 43 ± 6 | 390 ± 40 | 0.11 ± 0.01 |
| Glycerol | 99 ± 8 | 76.2 ± 1.8 | 1.30 ± 0.08 |
| L-Lactic acid | 260 ± 70 | 64 ± 4 | 4.1 ± 1.3 |
| Vitamin C | 320 ± 70 | 530 ± 50 | 0.60 ± 0.12 |
| Vitamin B6 | 57580 ± 9160 | 23.9 ± 0.8 | 2410 ± 400 |

TABLE 10
Kinetic constants for catecholamine interacting with a PBA. The PNRSS
measurements were performed as described in FIG. 43-48. A buffer of
1.5M KCl, 10 mM HEPES, pH 8.0 was used. A +160 mV potential was
continuously applied. For each measurement, kinetic constant values were
derived from events within a 15 min continuously recorded trace.
Three independent measurements (N = 3) were performed for
each condition to produce the statistics.

| Mobile Reactants | kₒₙ (M⁻¹s⁻¹) | k_{off} (s⁻¹) | K_{b} (M⁻¹) |
|---|---|---|---|
| Epinephrine | 9200 ± 900 | 1.09 ± 0.13 | 8300 ± 300 |
| Norepinephrine | 6000 ± 1000 | 0.98 ± 0.11 | 6200 ± 1500 |
| Isoprenaline | 4600 ± 1100 | 0.63 ± 0.02 | 7000 ± 2000 |

TABLE 11
Statistics of Δ̅I̅ and τ_{off} of catecholamine events. The PNRSS measurements
were performed as described in FIG. 43, FIG. 45 and FIG. 47. A buffer of
1.5M KCl, 10 mM HEPES, pH 8.0 was applied. Each mobile reactant
was added to trans with a final concentration of 140 μM. A +160 mV bias
was continuously applied during the measurement. For each measurement,
Δ̅I̅ and τ_{off} values were derived from events generated from a 5
min continuously recorded trace. Three independent measurements
(N = 3) were performed for each condition to produce the statistics.

| Mobile Reactants | Δ̅I̅ (pA) | τ_{off} (ms) |
|---|---|---|
| Norepinephrine | −21 ± 2 | 1020 ± 110 |
| Epinephrine | −25.9 ± 0.5 | 930 ± 110 |
| Isoprenaline | −32.2 ± 0.8 | 1580 ± 50 |

TABLE 12
Statistics of Δ̅I̅ and τ_{off} of remdesivir and remdesivir metabolite events. The
PNRSS measurements were performed as described in FIG. 53 and FIG. 55.
Remdesivir or remdesivir metabolite were added to trans with a final
concentration of 80 μM or 500 μM in separate, independent measurements.
A +160 mV bias was continuously applied during the measurement.
For each measurement, Δ̅I̅ and τ_{off} values were derived from events
generated from a 5 min continuously recorded trace. Three independent
measurements (N = 3) were performed for each condition to
form the statistics.

| Mobile Reactants | ΔI (pA) | τ_{off} (ms) |
|---|---|---|
| Remdesivir | 26.3 ± 0.6 | 100 ± 50 |
| Remdesivir Metabolite | 26.6 ± 1.5 | 22 ± 7 |

TABLE 13
Kinetic constants remdesivir and remdesivir metabolite binding to a PBA.
The PNRSS measurements were performed as described in FIG. 53
and FIG. 55. A buffer of 1.5M KCl, 10 mM HEPES, pH 8.0 was used. A
+160 mV potential was continuously applied. For each measurement,
kinetic constant values were derived from events within a 15 min
continuously recorded trace. Three independent measurements (N = 3)
were performed for each condition to produce the statistics.

| Mobile Reactants | kₒₙ (M⁻¹s⁻¹) | k_{off} (s⁻¹) | K_{b} (M⁻¹) |
|---|---|---|---|
| Remdesivir | 3760 ± 1270 | 1.03 ± 0.05 | 3610 ± 1070 |
| Remdesivir metabolite | 440 ± 30 | 47 ± 13 | 10 ± 2 |

TABLE 14
limit of detection. In this paper, the limit of detection is defined
as the minimum concentration of the analyte required so that at
least 5 events were detected within 10 minutes of measurement.
All PNRSS measurements were performed as described in Methods. A
buffer of 1.5M KCl, 10 mM HEPES, pH 7.0 was used and a
+180 mV potential was continuously applied for all measurements
with 14A, 14G, 13G/14G or 14TAZ. A buffer of 1.5M KCl, 10 mM
HEPES, pH 8.0 was used and a +160 mV potential was continuously
applied for all measurements with 14PBA.

| PNRSS strand | Mobile Reactants | limit of detection (μM) |
|---|---|---|
| 14A | Ni²⁺ | 0.2 |
| 14G | Ni²⁺ | 0.2 |
| 13G/14G | Ni²⁺ | 0.2 |
| 14TAZ | Ni²⁺ | 1 |
| | Co²⁺ | 1 |
| 14PBA | Catechol | 2 |
| | Ethylene glycol | 200 |
| | Glycerol | 150 |
| | L-Lactic acid | 60 |
| | Vitamin C | 40 |
| | Vitamin B6 | 0.4 |
| | Tris | 20 |
| | Epinephrine | 1 |
| | Norepinephrine | 1 |
| | Isoprenaline | 1 |
| | Remdesivir | 2 |
| | Remdesivir metabolite | 20 |

TABLE 15
Summary of binding constants κ_{b} (M⁻¹) between previous reports and
PNRSS.

| | | Previous | Binding | Binding | |
|---|---|---|---|---|---|
| | | Reports | Constants | Constants | |
| | | On | κ_{g} (M⁻¹) | κ_{b} (M⁻¹) | |
| Fixed | Mobile | Binding | In Previous | In PNRSS | Origin of |
| Reactant | Reactant | Constants | Reports | Measurements | Discussions |
| PBA | 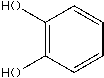 | Axthelm J, Askes SHC, Elstner M, G UR, Görls H, Bellstedt P, J. Am. Chem. Soc. 139, 11413-11420 (2017). | 3980 ± 2370 | 1100 ± 300 | Fig. 3b |
| | 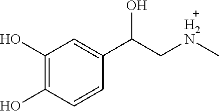 | Secor KE, Glass TE, Org. Lett. 6, 3727-3730 (2004). | 5000 | 8300 ± 300 | Fig. 5b |
| | 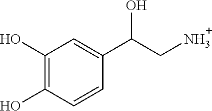 [SMILES: [NH3+]CC(O)c1ccc(O)c(O)c1] | Secor KE, Glass TE, Org. Lett. 6, 3727-3730 (2004). | 6500 | 6200 ± 1500 | Fig. 5b |
| | 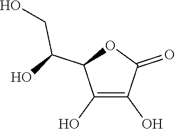 [SMILES: O=C1O[C@H]([C@@H](O)CO)C(O)=C1O] | Ali SR, Parajuli RR, Ma Y, Balogun Y, He H, J. Phys. Chem. B. 111, 12275-12281 (2007). | 21 ± 1.8 | 0.60 ± 0.12 | Fig. 3f |

TABLE 16
Summary of PNRSS measurements in this manuscript.

| | | | Previous | Previous | |
|---|---|---|---|---|---|
| | | | Reports On | Reports In | |
| | Fixed | Mobile | Chemical | Single | Origin of |
| Index | Reactant | Reactant | Reaction | Molecule | Discussions |
| 1 | 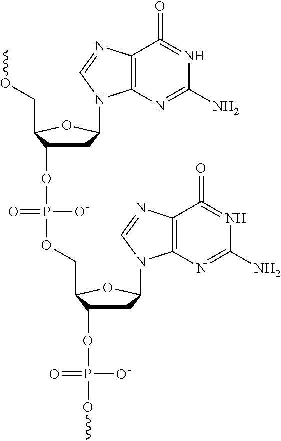 | Ni²⁺ | C. M. Mikulski, L. Mattucci, Y. Smith, T. B. Tran, N. M. Karayannis, Inorganica Chimica Acta 80, 127-133 (1983). | N/A | Fig. 1d |
| 2 | 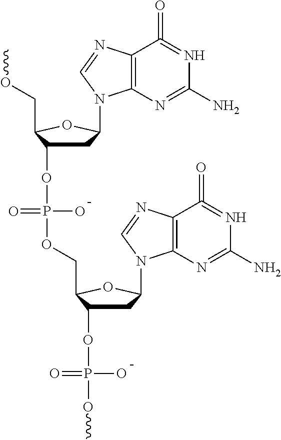 | Co²⁺ | M. S. Masoud, A. A. Soayed, A. E. Ali, Spectrochim. Acta, Part A, 60, 1907-1915 (2004). | N/A | Fig. 12d |
| 3 | 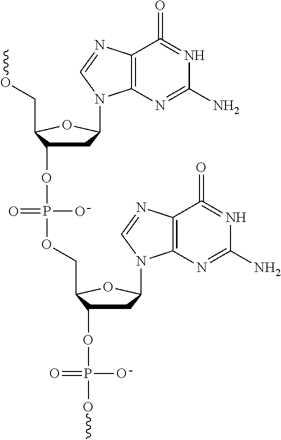 | Cu²⁺ | M. S. Masoud, A. A. Soayed, A. E. Ali, Spectrochim. Acta, Part A, 60, 1907-1915 (2004). | N/A | Fig. 12e |
| 4 | 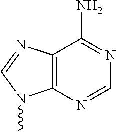 | Ni²⁺ | F. Huq, M. C. R. Peter, J. Inorg. Biochem. 78, 217-226 (2000). | N/A | Fig. 13 |
| 5 | 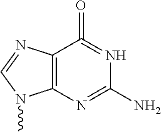 | Ni²⁺ | F. Huq, M. C. R. Peter, J. Inorg. Biochem. 78, 217-226 (2000). | N/A | Fig. 15 |
| 6 | 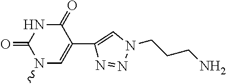 | Ni²⁺ | B. Schulze, U. S. Schubert, Chem. Soc. Rev.43, 2522-2571 (2014). | N/A | Fig. 2b |
| 7 | 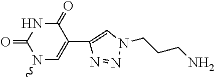 | Co²⁺ | Y. Fu et al., Chin. J. Chem. 28, 2226-2232 (2010). | N/A | Fig. 21 |
| 8 | 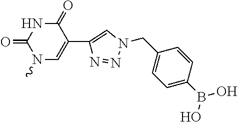 | 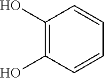 | J. P. Lorand, J. O. Edwards, J. Org. Chem.24, 769-774 (1959). | W. J. Bayley, Angew. Chem. Int. Ed.57, 2841-2845 (2018). | Fig. 3b |
| 9 | 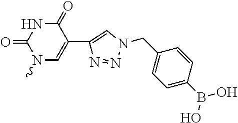 | 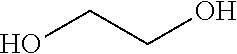 | J. P. Lorand, J. O. Edwards, J. Org. Chem.24, 769-774 (1959). | N/A | Fig. 3c |
| 10 | 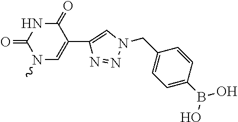 | 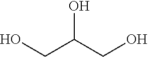 | J. P. Lorand, J. O. Edwards, J. Org. Chem.24, 769-774 (1959). | N/A | Fig. 3d |
| 11 | 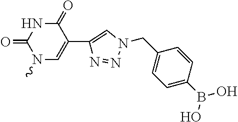 | 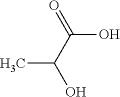 | E. Watanabe et al., Inorg. Chem. Commun. 13, 1406-1409 (2010). | N/A | Fig. 3e |
| 12 | 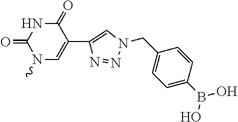 | 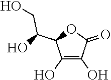 [SMILES: O=C1O[C@H]([C@@H](O)CO)C(O)=C1O] | D. A. Koese, B. Zuemreoglu- Karan, New J. Chem.33, 1874-1881 (2009). | N/A | Fig. 3f |
| 13 | 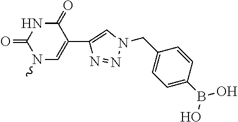 | 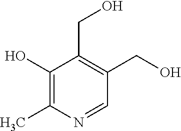 | D. A. Kose, B. Zumreoglu- Karan, O. Sahin, O. Buyukgungor, Inorg. Chim. Acta 413, 77-83 (2014). | N/A | Fig. 3g |
| 14 | 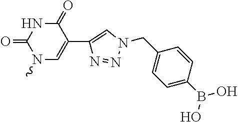 | 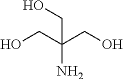 | N/A | N/A | Fig.39-41 |
| 15 | 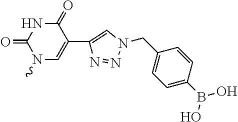 | H₂O₂ | A. Dutta, A. A. Ali, D. Sarma, J. Iran Chem. Soc. 16, 2379-2388 (2019). | N/A | Fig. 4b |
| 16 | 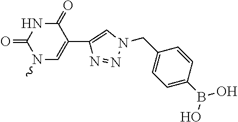 | 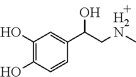 | S. Zhang, Y. Tang, Y. Chen, J. Zhang, Y. Wei, Microchimica Acta 187, (2020). | N/A | Fig. 5b |
| 17 | 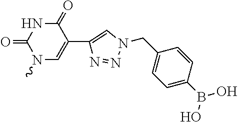 | 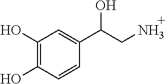 [SMILES: NCC(O)c1ccc(O)c(O)c1] | T. Ptak, P. Mlynarz, A. Dobosz, A. Rydzewska, M. Prokopowicz, J. Mol. Struct. 1040, 59-64 (2013). | N/A | Fig. 5b |
| 18 | 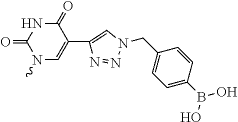 | 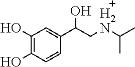 | S. Zhang, Y. Tang, Y. Chen, J. Zhang, Y. Wei, Microchimica Acta 187, (2020). | N/A | Fig. 5b |
| 19 | 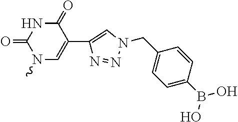 | 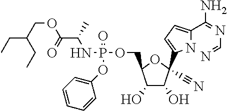 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1] | N/A | N/A | Fig. 6b |
| 20 | 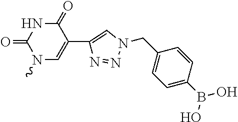 | 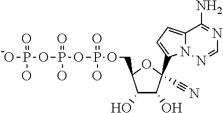 [SMILES: N#C[C@@]1(c2ccc3c(N)ncnn23)O[C@H](COP(=O)(O)OP(=O)(O)OP(=O)(O)O)[C@@H](O)[C@H]1O] | N/A | N/A | Fig. 6b |

Video 1| Ni²⁺ binding to a dual guanine reactant. The PNRSS measurement was carried out as described in FIG. 1. The electrolyte buffer applied was 1.5 M KCl, 10 mM HEPES, pH 7.0. The PNRSS strand 13G/14G (Table 1) was added to cis with a 10 nM final concentration. The dual guanine on 13G/14G serves as the fixed reactant. Ni²⁺, serving as the mobile reactant, was added to trans with a 1 mM final concentration. A +180 mV potential was continuously applied. Initially (the first ˜0.1 s), the pore was unoccupied and an open pore current was reported (˜575 pA). Afterwards, a PNRSS strand was captured and an immediate drop of current to ˜170 pA was observed. Further binding events measuring around −60 pA in ΔI successively appeared (0.2-5 s). These events result from reversible binding of Ni²⁺ to a dual guanine reactant, as explained by FIGS. 1c-1d.
Video 2| Ni²⁺ binding to a TAZ. The PNRSS measurement was carried out as described in FIG. 2. The electrolyte buffer applied was 1.5 M KCl, 10 mM HEPES, pH 7.0. The PNRSS strand 14TAZ (Table 1) was added to cis with a 10 nM final concentration. The TAZ serves as the fixed reactant. Ni²⁺, serving as the mobile reactant, was added to trans with a 1 mM final concentration. In this video, a PNRSS strand 14TAZ has already been captured by the pore. Events resulted from reversible binding of Ni²⁺ to a TAZ were continuously observed as further blockages. All events report characteristic noisy fluctuations when a Ni²⁺ was bound.
Video 3| Repetitive measurements of irreversible reactions. The PNRSS measurement was carried out as described in FIG. 4. The electrolyte buffer applied was 1.5 M KCl, 10 mM HEPES, pH 8.0. The PNRSS strand 14PBA (Table 1) was added to cis with a 10 nM final concentration. The phenylboronic acid (PBA) on a 14PBA serves as the fixed reactant. Hydrogen peroxide, serving as the mobile reactant, was added to trans with a 5.4 mM final concentration. Hydrogen peroxide could either reversibly bind to a PBA or irreversibly oxidize it to a phenol (FIGS. 4a, 4b). Initially, reversible binding of hydrogen peroxide to a PBA was continuously observed, reporting spiky, positive going events (2-80 s). Irreversible oxidation of PBA, which has inactivated the chemical reactivity of the fixed reactant, results in an event-free segment of the trace. By sequentially switching the applied potential to −100 mV (strand ejection) and +160 mV (strand reloading), the inactivated PNRSS strand was ejected and another reactive strand was loaded. By this special measurement mode of PNRSS, repetitive measurements of irreversible reactions can be carried out.
Video 4| Positive and negative going events acquired with a PBA. The PNRSS measurement was carried out as described in FIG. 10. The electrolyte buffer applied was 1.5 M KCl, 10 mM HEPES, pH 8.0. The PNRSS strand 14PBA (Table S1) was added to cis with a 10 nM final concentration. The phenylboronic acid (PBA) on a 14PBA serves as the fixed reactant. Catechol or norepinephrine, acting as the mobile reactant, was added to trans with a 280 μM final concentration for each analyte. In this video, a PNRSS strand 14PBA was captured by the pore, reporting an Iₚ value of ˜100 pA. Binding of catechol or norepinephrine respectively report positive (I_{b}>Iₚ) or negative (I_{b}<Iₚ) going events. Binding of catechol or norepinephrine was respectively labelled with C or N on the trace.
Video 5| PNRSS sensing of epinephrine, norepinephrine and isoprenaline. The PNRSS measurement was carried out as described in FIG. 5. The electrolyte buffer applied was 1.5 M KCl, 10 mM HEPES, pH 8.0. The PNRSS strand 14PBA (Table 1) was added to cis with a 10 nM final concentration. The phenylboronic acid (PBA) on a 14PBA serves as the fixed reactant. Norepinephrine, epinephrine and isoprenaline, acting as the mobile reactant, were added to trans with a 280 μM, a 280 μM and a 180 μM final concentration respectively. A +160 mV potential was continuously applied. In this video, a PNRSS strand 14PBA was captured by the pore. Successive binding of norepinephrine, epinephrine or isoprenaline to a PBA were observed. The raw trace was frequency split into the low pass (lp, the top trace) and the high pass (hp, the bottom trace) portion. A Butterworth filter was applied to perform the frequency split. The cut off frequency was set to 100 Hz (FIG. 50). Event identification was carried out by a machine learning algorithm (FIG. 51) and the identified events were labelled as N (norepinephrine), E (epinephrine) or I (isoprenaline) respectively.
Video 6| PNRSS sensing of remdesivir and its metabolite. The PNRSS measurement was carried out as described in FIG. 6. The electrolyte buffer applied was 1.5 M KCl, 10 mM HEPES, pH 8.0. The PNRSS strand 14PBA (Table 1) was added to cis with a 10 nM final concentration. The phenylboronic acid (PBA) on a 14PBA serves as the fixed reactant. Remdesivir and its metabolite, serving as the mobile reactant, were added to trans with a 20 μM or a 500 μM final concentration respectively. A +160 mV potential was continuously applied. Since the beginning of this video, a PNRSS strand 14PBA has already been captured by the pore. Binding of remdesivir or its metabolite to a PBA both results in positive going events (FIG. 6b). According to their different binding characteristics (FIG. 6b-d), remdesivir or its metabolite was identified and respectively labelled with R or M on the trace.
Video 7| An artistic demonstration of PNRSS. The core concept of PNRSS is to lower the technical hurdle of protein engineering to prepare a heterooligomeric nanopore nanoreactor. With PNRSS, the reactive component is used as separate modules in a toolbox. New applications are enabled by countless combinations of these modules. Repetitive engineering of the protein nanopore is however not required.

### REFERENCES

1. Huang, S. et al. Identifying single bases in a DNA oligomer with electron tunnelling. Nature Nanotechnology 5, 868-873 (2010).
2. Zrimsek, A. B. et al. Single-Molecule Chemistry with Surface- and Tip-Enhanced Raman Spectroscopy. Chemical Reviews 117, 7583-7613 (2017).
3. Jia, C. et al. Covalently bonded single-molecule junctions with stable and reversible photoswitched conductivity. Science 352, 1443-1445 (2016).
4. Huang, W. et al. Maleimide-thiol adducts stabilized through stretching. Nature Chemistry 11, 310-319 (2019).
5. Braha, O. et al. Designed protein pores as components for biosensors. Chemistry & Biology 4, 497-505 (1997).
6. Song, L. et al. Structure of Staphylococcal α-Hemolysin, a Heptameric Transmembrane Pore. Science 274, 1859-1866 (1996).
7. Butler, T. Z., Pavlenok, M., Derrington, I. M., Niederweis, M. & Gundlach, J. H. Single-molecule DNA detection with an engineered MspA protein nanopore. P Natl Acad Sci USA 105, 20647-20652 (2008).
8. Cao, C. et al. Discrimination of oligonucleotides of different lengths with a wild-type aerolysin nanopore. Nature Nanotechnology 11, 713-718 (2016).
9. Bock, A. V., Dill, D. B. & Edwards, H. T. LACTIC ACID IN THE BLOOD OF RESTING MAN. The Journal of clinical investigation 11, 775-788 (1932).
10. Soskine, M. et al. An Engineered ClyA Nanopore Detects Folded Target Proteins by Selective External Association and Pore Entry. Nano Letters 12, 4895-4900 (2012).
11. Wloka, C., Mutter, N. L., Soskine, M. & Maglia, G. Alpha-Helical Fragaceatoxin C Nanopore Engineered for Double-Stranded and Single-Stranded Nucleic Acid Analysis. Angewandte Chemie International Edition 55, 12494-12498 (2016).
12. Huang, G. et al. Electro-Osmotic Vortices Promote the Capture of Folded Proteins by PlyAB Nanopores. Nano Letters 20, 3819-3827 (2020).
13. Fahie, M. A., Yang, B., Pham, B. & Chen, M. Tuning the Selectivity and Sensitivity of an OmpG Nanopore Sensor by Adjusting Ligand Tether Length. ACS Sensors 1, 614-622 (2016).
14. Wendell, D. et al. Translocation of double-stranded DNA through membrane-adapted phi29 motor protein nanopores. Nature Nanotechnology 4, 765-772 (2009).
15. Stoddart, D., Heron, A. J., Mikhailova, E., Maglia, G. & Bayley, H. Single-nucleotide discrimination in immobilized DNA oligonucleotides with a biological nanopore. Proceedings of the National Academy of Sciences 106, 7702-7707 (2009).
16. Stefureac, R., Long, Y. T., Kraatz, H. B., Howard, P. & Lee, J. S. Transport of alpha-helical peptides through alpha-hemolysin and aerolysin pores. Biochemistry 45, 9172-9179 (2006).
17. Van Meervelt, V. et al. Real-Time Conformational Changes and Controlled Orientation of Native Proteins Inside a Protein Nanoreactor. Journal of the American Chemical Society 139, 18640-18646 (2017).
18. Galenkamp, N. S., Biesemans, A. & Maglia, G. Directional conformer exchange in dihydrofolate reductase revealed by single-molecule nanopore recordings. Nature Chemistry 12, 481-488 (2020).
19. Quick, J. et al. Real-time, portable genome sequencing for Ebola surveillance. Nature 530, 228-232 (2016).
20. Roozbahani, G. M., Chen, X., Zhang, Y., Wang, L. & Guan, X. Nanopore Detection of Metal Ions: Current Status and Future Directions. Small Methods 4, 2000266 (2020).
21. Kasianowicz, J. J., Burden, D. L., Han, L. C., Cheley, S. & Bayley, H. Genetically Engineered Metal Ion Binding Sites on the Outside of a Channel's Transmembrane β-Barrel. Biophysical Journal 76, 837-845 (1999).
22. Choi, L.-S., Mach, T. & Bayley, H. Rates and Stoichiometries of Metal Ion Probes of Cysteine Residues within Ion Channels. Biophysical Journal 105, 356-384 (2013).
23. Braha, O. et al. Simultaneous stochastic sensing of divalent metal ions. Nature Biotechnology 18, 1005-1007 (2000).
24. Borsley, S. & Cockroft, S. L. In Situ Synthetic Functionalization of a Transmembrane Protein Nanopore. ACS Nano 12, 786-794 (2018).
25. Zhou, B., Wang, Y.-Q., Cao, C., Li, D.-W. & Long, Y.-T. Monitoring disulfide bonds making and breaking in biological nanopore at single molecule level. Science China Chemistry 61, 1385-1388 (2018).
26. Haugland, M. M., Borsley, S., Cairns-Gibson, D. F., Elmi, A. & Cockroft, S. L. Synthetically Diversified Protein Nanopores: Resolving Click Reaction Mechanisms. ACS Nano 13, 4101-4110 (2019).
27. Cao, J. et al. Giant single molecule chemistry events observed from a tetrachloroaurate(III) embedded Mycobacterium smegmatis porin A nanopore. Nature Communications 10, 5668 (2019).
28. Lee, J. & Bayley, H. Semisynthetic protein nanoreactor for single-molecule chemistry. P Natl Acad Sci USA 112, 13768-13773 (2015).
29. Lee, J. et al. Semisynthetic Nanoreactor for Reversible Single-Molecule Covalent Chemistry. Acs Nano 10, 8843-8850 (2016).
30. Bétermier, F. et al. Single-sulfur atom discrimination of polysulfides with a protein nanopore for improved batteries. Communications Materials 1, 59 (2020).
31. Clarke, J. et al. Continuous base identification for single-molecule nanopore DNA sequencing. Nature Nanotechnology 4, 265-270 (2009).
32. Zernia, S., van der Heide, N.J., Galenkamp, N. S., Gouridis, G. & Maglia, G. Current Blockades of Proteins inside Nanopores for Real-Time Metabolome Analysis. ACS Nano 14, 2296-2307 (2020).
33. Wallace, E. V. B. et al. Identification of epigenetic DNA modifications with a protein nanopore. Chemical Communications 46, 8195-8197 (2010).
34. Manrao, E. A., Derrington, I. M., Pavlenok, M., Niederweis, M. & Gundlach, J. H. Nucleotide Discrimination with DNA Immobilized in the MspA Nanopore. Plos One 6 (2011).
35. Hogrefe, R. I., Midthune, B. & Lebedev, A. Current Challenges in Nucleic Acid Synthesis. Israel Journal of Chemistry 53, 326-349 (2013).
36. Huq, F. & Peter, M. C. R. Interaction between NiCl2, and nucleobases, nucleosides and nucleotides. Journal of Inorganic Biochemistry 78, 217-226 (2000).
37. Masoud, M. S., Soayed, A. A. & Ali, A. E. Complexing properties of nucleic-acid constituents adenine and guanine complexes. Spectrochimica Acta Part A: Molecular and Biomolecular Spectroscopy 60, 1907-1915 (2004).
38. EI-Sagheer, A. H. & Brown, T. Click chemistry with DNA. Chemical Society Reviews 39, 1388-1405 (2010).
39. Kolb, H. C., Finn, M. G. & Sharpless, K. B. Click Chemistry: Diverse Chemical Function from a Few Good Reactions. Angewandte Chemie International Edition 40, 2004-2021 (2001).
40. Struthers, H., Spingler, B., Mindt, T. L. & Schibli, R. “Click-to-Chelate”: Design and Incorporation of Triazole-Containing Metal-Chelating Systems into Biomolecules of Diagnostic and Therapeutic Interest. Chemistry—A European Journal 14, 6173-6183 (2008).
41. Meng, G. et al. Modular click chemistry libraries for functional screens using a diazotizing reagent. Nature 574, 86-89 (2019).
42. Wu, X. et al. Selective sensing of saccharides using simple boronic acids and their aggregates. Chemical Society Reviews 42, 8032-8048 (2013).
43. Pizer, R. & Babcock, L. Mechanism of the complexation of boron acids with catechol and substituted catechols. Inorganic Chemistry 16, 1677-1681 (1977).
44. Babcock, L. & Pizer, R. Dynamics of boron acid complexation reactions. Formation of 1:1 boron acid-ligand complexes. Inorganic Chemistry 19, 56-61 (1980).
45. Pizer, R. & Tihal, C. Equilibria and reaction mechanism of the complexation of methylboronic acid with polyols. Inorganic Chemistry 31, 3243-3247 (1992).
46. Köse, D. A. & Zumreoglu-Karan, B. Complexation of boric acid with vitamin C. New Journal of Chemistry 33, 1874-1881 (2009).
47. Köse, D. A., Zumreoglu-Karan, B., Sahin, O. & Büyükgüngör, O. Boric acid complexes with thiamine (vitamin B1) and pyridoxine (vitamin B6). Inorganica Chimica Acta 413, 77-83 (2014).
48. Lorand, J. P. & Edwards, J. O. Polyol Complexes and Structure of the Benzeneboronate Ion. The Journal of Organic Chemistry 24, 769-774 (1959).
49. Bull, S. D. et al. Exploiting the Reversible Covalent Bonding of Boronic Acids: Recognition, Sensing, and Assembly. Accounts of Chemical Research 46, 312-326 (2013).
50. Ramsay, W. J., Bell, N. A. W., Qing, Y. & Bayley, H. Single-Molecule Observation of the Intermediates in a Catalytic Cycle. Journal of the American Chemical Society 140, 17538-17546 (2018).
51. Good, N. E. et al. Hydrogen Ion Buffers for Biological Research. Biochemistry 5, 467-477 (1966).
52. Ramsay, W. J. & Bayley, H. Single-Molecule Determination of the Isomers of d-Glucose and d-Fructose that Bind to Boronic Acids. Angewandte Chemie International Edition 57, 2841-2845 (2018).
53. Kuivila, H. G. Electrophilic Displacement Reactions. III. Kinetics of the Reaction between Hydrogen Peroxide and Benzeneboronic Acid. Journal of the American Chemical Society 76, 870-874 (1954).
54. Hao, L., Ding, G., Deming, D. A. & Zhang, Q. Recent Advances in Green Synthesis of Functionalized Phenols from Aromatic Boronic Compounds. European Journal of Organic Chemistry 2019, 7307-7321 (2019).
55. Dutta, A., Ali, A. A. & Sarma, D. Versatile catalysis of “natural extract”: oxidation of sulfides and alcohols and ipso-hydroxylation of arylboronic acids. Journal of the Iranian Chemical Society 16, 2379-2388 (2019).
56. Reiner, S., Ambrosio, M., Hoffmann, C. & Lohse, M. J. Differential Signaling of the Endogenous Agonists at the beta(2)-Adrenergic Receptor. Journal of Biological Chemistry 285, 36188-36198 (2010).
57. Moore, R. Y. & Bloom, F. E. Central Catecholamine Neuron Systems: Anatomy and Physiology of the Norepinephrine and Epinephrine Systems. Annual Review of Neuroscience 2, 113-168 (1979).
58. Nichtova, Z., Novotova, M., Kralova, E. & Stankovicova, T. Morphological and functional characteristics of models of experimental myocardial injury induced by isoproterenol. General Physiology and Biophysics 31, 141-151 (2012).
59. Cortes, C. & Vapnik, V. Support-vector networks. Machine Learning 20, 273-297 (1995).
60. Ptak, T., Mtynarz, P., Dobosz, A., Rydzewska, A. & Prokopowicz, M. Potentiometric and NMR complexation studies of phenylboronic acid PBA and its aminophosphonate analog with selected catecholamines. Journal of Molecular Structure 1040, 59-64 (2013).
61. Zhang, S., Tang, Y., Chen, Y., Zhang, J. & Wei, Y. Boronic acid-modified polyhedral oligomeric silsesquioxanes on polydopamine-coated magnetized graphene oxide for selective and high-capacity extraction of the catecholamines epinephrine, dopamine and isoprenaline. Microchimica Acta 187, 77 (2020).
62. Boersma, A. J., Brain, K. L. & Bayley, H. Real-Time Stochastic Detection of Multiple Neurotransmitters with a Protein Nanopore. ACS Nano 6, 5304-5308 (2012).
63. Secor, K. E. & Glass, T. E. Selective Amine Recognition: Development of a Chemosensor for Dopamine and Norepinephrine. Organic Letters 6, 3727-3730 (2004).
64. Zhang, L., Liu, X. A., Gillis, K. D. & Glass, T. E. A High-Affinity Fluorescent Sensor for Catecholamine: Application to Monitoring Norepinephrine Exocytosis. Angewandte Chemie International Edition 58, 7611-7614 (2019).
65. Flatmark Catecholamine biosynthesis and physiological regulation in neuroendocrine cells. Acta Physiologica Scandinavica 168, 1-17 (2000).
66. Seley-Radtke, K. L. & Yates, M. K. The evolution of nucleoside analogue antivirals: A review for chemists and non-chemists. Part 1: Early structural modifications to the nucleoside scaffold. Antiviral Research 154, 66-86 (2018).
67. Warren, T. K. et al. Therapeutic efficacy of the small molecule GS-5734 against Ebola virus in rhesus monkeys. Nature 531, 381-385 (2016).
68. Holshue, M. L. et al. First Case of 2019 Novel Coronavirus in the United States. New England Journal of Medicine 382, 929-936 (2020).
69. McMahon, J. H., Udy, A. & Peleg, A. Y. Remdesivir for the Treatment of Covid-19—Preliminary Report. New England Journal of Medicine 383, 992-993 (2020).
70. Wang, M. et al. Remdesivir and chloroquine effectively inhibit the recently emerged novel coronavirus (2019-nCoV) in vitro. Cell Research 30, 269-271 (2020).
71. Yin, W. et al. Structural basis for inhibition of the RNA-dependent RNA polymerase from SARS-CoV-2 by remdesivir. Science 368, 1499-1504 (2020).
72. Martin, A. R., Vasseur, J.-J. & Smietana, M. Boron and nucleic acid chemistries: merging the best of both worlds. Chemical Society Reviews 42, 5684-5713 (2013).
73. Beigel, J. H. et al. Remdesivir for the Treatment of Covid-19—Final Report. New England Journal of Medicine (2020).
74. Pan, H. et al. Repurposed antiviral drugs for COVID-19-interim WHO SOLIDARITY trial results. medRxiv, 2020.2010.2015.20209817 (2020).
75. Warren, T. K. et al. Protection against filovirus diseases by a novel broad-spectrum nucleoside analogue BCX4430. Nature 508, 402-405 (2014).
76. Oxford, J. S. Inhibition of the Replication of Influenza A and B Viruses by a Nucleoside Analogue (Ribavirin). Journal of General Virology 28, 409-414 (1975).
77. Furuta, Y. et al. Favipiravir (T-705), a novel viral RNA polymerase inhibitor. Antiviral Research 100, 446-454 (2013).
78. Peleg, M., Normand, M. D. & Corradini, M. G. The Arrhenius Equation Revisited. Critical Reviews in Food Science and Nutrition 52, 830-851 (2012).
79. Ross, G. A. et al. Plasma and 24 H-Urinary Catecholamine Concentrations in Normal and Patient Populations. Annals of Clinical Biochemistry 30, 38-44 (1993).
80. Fahie, M. A., Yang, B., Mullis, M., Holden, M. A. & Chen, M. Selective Detection of Protein Homologues in Serum Using an OmpG Nanopore. Analytical Chemistry 87, 11143-11149 (2015).
81. Galenkamp, N. S., Soskine, M., Hermans, J., Wloka, C. & Maglia, G. Direct electrical quantification of glucose and asparagine from bodily fluids using nanopores. Nature Communications 9, 4085 (2018).
82. Patterson, D. M., Nazarova, L. A. & Prescher, J. A. Finding the Right (Bioorthogonal) Chemistry. ACS Chemical Biology 9, 592-605 (2014).
83. Wang, Y. et al. Osmosis-Driven Motion-Type Modulation of Biological Nanopores for Parallel Optical Nucleic Acid Sensing. ACS Applied Materials & Interfaces 10, 7788-7797 (2018).
84. Stoddart, D., Heron, A. J., Mikhailova, E., Maglia, G. & Bayley, H. Single-nucleotide discrimination in immobilized DNA oligonucleotides with a biological nanopore. Proceedings of the National Academy of Sciences 106, 7702-7707 (2009).
85. Frisch, M. J. et al. (Wallingford, CT; 2016).
86. Zhao, Y. & Truhlar, D. G. The M06 suite of density functionals for main group thermochemistry, thermochemical kinetics, noncovalent interactions, excited states, and transition elements: two new functionals and systematic testing of four M06-class functionals and 12 other functionals. Theoretical Chemistry Accounts 120, 215-241 (2008).
87. Zhao, Y. & Truhlar, D. G. Density Functionals with Broad Applicability in Chemistry. Accounts of Chemical Research 41, 157-167 (2008).
88. Wadt, W. R. & Hay, P. J. Ab initio effective core potentials for molecular calculations. Potentials for main group elements Na to Bi. The Journal of Chemical Physics 82, 284-298 (1985).
89. Hinkes, S. P. A. & Klein, C. D. P. Virtues of Volatility: A Facile Transesterification Approach to Boronic Acids. Organic Letters 21, 3048-3052 (2019).
90. Zhou, J., Stapleton, P., Haider, S. & Healy, J. Boronic acid inhibitors of the class A β-lactamase KPC-2. Bioorganic & Medicinal Chemistry 26, 2921-2927 (2018).
91. Masoud, M. S., Soayed, A. A. & Ali, A. E. Complexing properties of nucleic-acid constituents adenine and guanine complexes. Spectrochimica Acta Part A: Molecular and Biomolecular Spectroscopy 60, 1907-1915 (2004).
92. Huq, F. & Peter, M. C. R. Interaction between NiCl2, and nucleobases, nucleosides and nucleotides. Journal of Inorganic Biochemistry 78, 217-226 (2000).
93. Mai, K., Lin, J., Zhuang, B., Li, X. & Zhang, L.-M. Cationic dendronization of amylose via click chemistry for complexation and transfection of plasmid DNA. International Journal of Biological Macromolecules 79, 209-216 (2015).
94. Struthers, H., Spingler, B., Mindt, T. L. & Schibli, R. “Click-to-Chelate”: Design and Incorporation of Triazole-Containing Metal-Chelating Systems into Biomolecules of Diagnostic and Therapeutic Interest. Chemistry—A European Journal 14, 6173-6183 (2008).
95. Steinmeyer, J. & Wagenknecht, H.-A. Synthesis of DNA Modified with Boronic Acid: Compatibility to Copper(I)-Catalyzed Azide-Alkyne Cycloaddition. Bioconjugate Chemistry 29, 431-436 (2018).
96. Dai, C. et al. The first chemical synthesis of boronic acid-modified DNA through a copper-free click reaction. Chemical Communications 47, 3598-3600 (2011).
97. Pizer, R. & Babcock, L. Mechanism of the complexation of boron acids with catechol and substituted catechols. Inorganic Chemistry 16, 1677-1681 (1977).
98. Pizer, R. & Tihal, C. Equilibria and reaction mechanism of the complexation of methylboronic acid with polyols. Inorganic Chemistry 31, 3243-3247 (1992).
99. Köse, D. A. & Zümreoglu-Karan, B. Complexation of boric acid with vitamin C. New Journal of Chemistry 33, 1874-1881 (2009).
100. Köse, D. A., Zumreoglu-Karan, B., Sahin, O. & Büyükgüngör, O. Boric acid complexes with thiamine (vitamin B1) and pyridoxine (vitamin B6). Inorganica Chimica Acta 413, 77-83 (2014).
101. Zhang, S., Tang, Y., Chen, Y., Zhang, J. & Wei, Y. Boronic acid-modified polyhedral oligomeric silsesquioxanes on polydopamine-coated magnetized graphene oxide for selective and high-capacity extraction of the catecholamines epinephrine, dopamine and isoprenaline. Microchimica Acta 187, 77 (2020).
102. Qi, F. et al. arXiv:1901.08013 (2018).
103. Martin, A. R., Vasseur, J.-J. & Smietana, M. Boron and nucleic acid chemistries: merging the best of both worlds. Chemical Society Reviews 42, 5684-5713 (2013).
104. Peleg, M., Normand, M. D. & Corradini, M. G. The Arrhenius Equation Revisited. Critical Reviews in Food Science and Nutrition 52, 830-851 (2012).

1. A system for characterizing a target analyte, comprising:
a nanopore; and
a polymer strand comprising a tether site and a reaction section,
wherein the polymer strand is tethered via the tether site so that the polymer strand cannot pass through the nanopore, and wherein the reaction section comprises at least one sensing module which can interact with single molecule of the target analyte.

2. The system according to claim 1, wherein the reaction section comprises two or more sensing modules which can interact with two or more different target analytes, and wherein each sensing module consists of one, two or more sensing moieties and each sensing moiety can interact with one or two or more binding sites of single molecule of the target analyte.

3. (canceled)

4. The system according to claim 2, wherein the sensing moiety is selected from the group consisting of base of any nucleotide, any amino acid, 1,2,3-trizole, phenylboronic acid (PBA) or any combination thereof.

5. The system according to claim 1, wherein at least one of the sensing modules consists of two neighbouring purines selected from the group consisting of guanine and adenine.

6. (canceled)

7. (canceled)

8. (canceled)

9. The system according to claim 1, wherein the polymer strand is tethered to a stopper molecule or the nanopore protein.

10. The system according to claim 9, wherein the stopper molecule is a protein which can specifically bind a small molecule compound, the tether site comprises the small molecule compound, and the polymer strand is tethered to the stopper molecule through the specific binding of the small molecule compound to the protein; or the stopper molecule is streptavidin or an antibody of a hapten and the small molecule compound is biotin or the hapten.

11. (canceled)

12. The system according to claim 9, wherein the tether site comprises a small molecule that can react with a natural amino acid on the surface of the stopper molecule or the nanopore protein, and the polymer strand is tethered to the stopper molecule through the reaction between the small molecule compound and the natural amino acid.

13. The system according to claim 9, wherein a first reactive handle is introduced to the surface of the stopper molecule or the nanopore protein, the tether site comprises a second reactive handle, and the polymer strand is tethered to the stopper molecule through the reaction between the first reactive handle and the second reactive handle.

14. The system according to claim 1, wherein the polymer strand further comprises an extension section and the extension section is configured to enable the reaction section to be located in a region suitable for measurement of a blockage.

15. The system according to claim 1, wherein the polymer strand further comprises a traction section and the traction section is configured to hold the reaction section in a region suitable for measurement of a blockage.

16. The system according to claim 15, wherein the traction section comprises any one of the following:
a. a polymer chain which tend to pass through the nanopore channel in the electric field applied to the nanopore;
b. a coupling site which can react with a natural amino acid on the surface of the channel of the nanopore;
c. a second reactive handle which can react with a first reactive introduced to the surface of the channel of the nanopore; or
d. a polymer chain that can pass through the channel of the nanopore and form a three-dimension structure outside the nanopore which has a size larger than the exit opening of the nanopore.

17. (canceled)

18. The system according to claim 1, wherein the polymer strand is based on nucleic acid, nucleic acid analog, polypeptide, polysaccharide, a homopolymer, a copolymer, or any combination thereof.

19. The system according to claim 1, wherein the target analyte is selected from the group consisting of:
ion comprising metal element;
monosaccharide;
oligosaccharide;
polysaccharide;
glucoside;
polyphenol;
catecholamine;
catecholamine derivative;
polyol;
protonated or deprotonated forms of a compound;
a compound containing a ribose moiety;
hydrogen peroxide;
oligopeptide or cyclopeptide;
buffer reagent;
smaller molecular drug;
neurotransmitter;
compound with a specific chirality;
chemical intermediate;
or any combination thereof.

20. The system according to claim 1, wherein the nanopore is a biological nanopore, a solid nanopore or a DNA nanopore.

21. The system according to claim 20, wherein the protein nanopore is MspA, α-HL, Aerolysin, ClyA, FhuA, FraC, PlyA/B, CsgG Phi 29 connector or a homolog or variant thereof.

22. (canceled)

23. Method for characterizing a target analyte, the method comprising:
(i) providing the system according to claim 1;
(ii) applying a voltage between the two sides of the nanopore and allowing one polymer strand to enter the nanopore;
(iii) allowing a target analyte to pass through the nanopore; and
(iv) measuring an ionic current through the nanopore to provide a current pattern, and characterizing the target analyte based on the current pattern.

24. The method according to claim 23, wherein the polymer strand of the system comprises two or more sensing modules which can interact with two or more different target analytes, and wherein the method is for characterizing two or more target analytes.

25. The method according to claim 23, wherein the method comprises:
(i) providing the system according to claim 1;
(ii) applying a first voltage between the two sides of the nanopore and allowing one polymer strand to enter the nanopore;
(iii) allowing a first target analyte to pass through the nanopore; and
(iv) measuring an ionic current through the nanopore to provide a current pattern, and characterizing the first target analyte based on the current pattern.
(v) switching the voltage between the two compartments to a second voltage which is in the opposite direction to the first voltage, thereby causing the polymer strand in the nanopore to exit from the nanopore;
(vi) switching the voltage between the two compartments to the first voltage and allowing another polymer strand to enter the nanopore; and
(vi) applying steps (iii)-(iv) to a second target analyte which is different from the first target analyte.

26. The according to claim 25, wherein the sensing module is capable of irreversibly interacting with the first target analyte and/or the second target analyte.

27. The method according to claim 23, wherein the target analyte is selected from the group consisting of:
ion comprising metal element;
monosaccharide;
oligosaccharide;
polysaccharide;
glucoside;
polyphenol;
catecholamine;
catecholamine derivative;
polyol;
protonated or deprotonated forms of a compound;
a compound containing a ribose moiety;
hydrogen peroxide;
oligopeptide or cyclopeptide;
buffer reagent;
smaller molecular drug;
neurotransmitter;
compound with a specific chirality;
analyte containing an isotope;
chemical intermediate;
or any combination thereof.
